SEQUENCE LISTING (56 sequences)

SEQ ID NO: 1 - DNA, 2149 bp, Homo sapiens
cagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtacaatgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttc

SEQ ID NO: 2 - DNA, 2044 bp, Mus musculus
ctgacgcgggcaggctccacgctgaacggttacacagagaggaaacaataaatctaagctactattgcaataaatatctcaagttttaacgaaggaaactatcattacagttaaaattttttaaagtaacgcttttttagaacaaagctaacaaatggctagttttctgtggatcttcttcaaacgctttctttaacggggaaagagtcaaacaagcagttttacctgaaataaagaactagtttaaaggtcagaagagaagagcaagctttgcaggaggcacggaaggcaagcgctggcagtacaatgacagttttcctttcctttgcattcttcgctgccattctgactcacatagggtgcagcaaccagcgccgaaatccagaaaacggagggagaagatataaccggattcaacatgggcaatgtgcctacactttcattcttccagaacacgacgggaactgccgtgagagtgcgacagagcagtacaacaccaacgctctgcaaagggatgctccacacgtggagccggatttctcttcccagaaacttcagcatctggagcatgtgatggaaaattatactcagtggctgcaaaaacttgagaattacattgtggaaaatatgaagtcggagatggcccagatacaacagaatgctgttcaaaaccacacggccaccatgcttgagataggaaccagtctcttatctcagactgcagagcagacccgaaagctgacagatgttgagacccaggtactaaatcaaacatcccgacttgaaatacaactgctagagaattcattatcaacatacaagctagagaagcaacttctccaacagacaaatgaaattctgaagattcacgaaaaaaacagtttactagagcacaaaatcttagaaatggagggaaaacacaaagaagaattggacaccttgaaggaggagaaagaaaaccttcaaggcttggtttctcgtcagacattcatcatccaggagttggagaagcaacttagtagagctaccaacaacaacagcatcctgcagaagcaacaactggagctcatggacacagttcataaccttgtcagcctttgcactaaagaaggtgttttgctaaagggaggaaaaagagaagaagagaaaccatttcgagactgtgcagatgtatatcaagctggttttaataaaagtggaatctacactatttattttaataatatgccagaacccaaaaaggtattttgcaatatggatgtgaatgggggaggttggacagtaatacaacaccgggaagatggaagcctggatttccagaggggctggaaggagtataaaatgggttttgggaatccctctggtgaatattggctcgggaacgagttcatttttgcaataaccagtcagaggcagtacatgctgaggattgagctgatggactgggaagggaaccgagcctactcacagtacgacagattccacataggaaatgaaaagcagaactataggttatatttaaaaggtcacacagggacagcaggcaaacagagcagcttgatcttacacggtgctgatttcagcacgaaggatgctgataacgacaactgtatgtgcaaatgcgctctcatgctaacaggaggttggtggttcgatgcctgtggcccttccaatctaaatggaatgttctacactgcgggacaaaatcatggaaaactgaatgggataaagtggcactacttcaaagggcccagttactccttacgttccaccaccatgatgatccggcccttggacttttgaaggtgctatgccagtattagaaagctgcaaagaaagctgggcatgttcccagatgagaagctagtcagaggcttcagaaacaaccaacattgtctccgttccagcagcaagtggttatgtcatgtcacctgggtacttaacaatggatttggagccttctgaggtcaacagaatcgccacttgggtccagagaatgccactcacaatcatgtttaaaagggaagaaacttctcagcttgct

SEQ ID NO: 3 - DNA, 1587 bp, Bos taurus
tctgtgattcttctacaaaactttctttgaaagggaaaagtcaaacaaacaagcagttttacctgaaataaagaactagtttaaaggtcagaagaaaggagcgagttttgcgagaggcacggaaagagagtgccggcagtacaatgacagttttcctttcctttgctttcctcgctgctattctgactcacatagggtgcagcaaccagcgccgaagtccagaaaacggtgggagaagatataaccggattcaacatgggcaatgtgcctacactttcattcttccagaacacgacggtaactgtcgtgagagtacgacagaccagtacaacacaaacgctctgcagagagatgctccacacgtggaacaggatttctcttcccagaaacttcaacatctggagcatgtgatggaaaattatactcagtggctgcaaaaaattgagaattacattgtggaaaatatgaagtcggagatggctcagatacagcagaatgcagttcagaaccacacggccaccatgctcgagatagggaccagcctcctctctcagaccgcagagcagaccagaaagctgacagatgtagagacccaggtactaaatcaaacatctcgacttgaaatacaactgctggagaattcattatcaacctacaagctagagaaacaacttcttcaacagacaaatgaaatcttaaagattcatgagaaaaacagtttattagaacacaaaatctttgaaatggagggaaagcacaaggaagaattggacaccttaaaggaagagaaagagaatcttcaaggcttggttactcgtcagacgtacataatccaggaactggagaaacagttgaacagagccaccaccaataacagtgtccttcagaagcagcagctggagctcatggacacagtgcacaacctcgtcaacctctgcactaaagaagttttactaaagggaggaaaaagaaaggaagaaaagccatttagagactgtgcagatgtatatcaagctggttttaataaaagtggaatctacactatttatattaataatatgccagaacccaaaaaggtattttgcgatatggatctcaacgggggaggttggactgtaatacagcatcgtgaagacggaagtctagatttccaaagaggttggaaagaatataaaatgggttttggaaatccctctggtgaatattggttggggaacgagtttatttttgccataaccagtcagaggcagtacactctaagaattgagttattggactgggaaggaaaccgagcctattcacagtacgacagattccacataggaaatgaaaagcagaactacaggttgtatttgaagggtcacactgggacagcaggaaaacagagcagcctgatcttgcatggtgctgatttcagcactaaagatgctgataacgacaactgcatgtgcaaatgtgccctcatgctaacaggaggatggtggtttgatgcctgtggcccatccaatctaaatggaatgttttatacagcgggacagaaccatggaaaactgaatggaataaaatggcattatttcaaagggccca

SEQ ID NO: 4 - DNA, 330 bp, RAT
gaaaattatactcagtggctggaaaaacttgagaattacattgtggaaaatatgaagtcggagatggcccagatacaacagaatgcggttcaaaaccacacggccaccatgctggagataggaaccagcctcttgtctcagactgcagagcagacccgaaagctcacagatgtggagacccaggtactaaatcaaacatcccgtcttgaaatccaactgctggagaattcattatcaacatacaagctagaaaaacagcttctccaacagacaaatgaaattctgaagattcagaaaaaaaacagtttattagagcataaaatcctagaa

SEQ ID NO: 5 - DNA, 330 bp, Bos taurus
gaaaattatactcagtggctgcaaaaaattgagaattacattgtggaaaatatgaagtcggagatggctcagatacagcagaatgcagttcagaaccacacggccaccatgctcgagatagggaccagcctcctctctcagaccgcagagcagaccagaaagctgacagatgtagagacccaggtactaaatcaaacatctcgacttgaaatacaactgctggagaattcattatcaacctacaagctagagaaacaacttcttcaacagacaaatgaaatcttaaagattcatgagaaaaacagtttattagaacacaaaatctttgaa

SEQ ID NO: 6 - DNA, 3041 bp, Homo sapiens
gaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggcgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaaccaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggatatatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttcatttaattgtttcagaataggataattaggtattcgaattttgtctttaaaattcatgtggtttctatgcaaagttcttcatatcatcacaacattatttgatttaaataaaattgaaagt

SEQ ID NO: 7 - DNA, 330 bp, RAT
gaaaattatactcagtggctggaaaaacttgagaattacattgtggaaaatatgaagtcggagatggcccagatacaacagaatgcggttcaaaaccacacggccaccatgctggagataggaaccagcctcttgtctcagactgcagagcagacccgaaagctcacagatgtggagacccaggtactaaatcaaacatcccgtcttgaaatccaactgctggagaattcattatcaacatacaagctagagaaacagcttctccaacagacaaatgaaattctgaagattcagaaaaaaaacagtttattggaacataaaatcctagaa

SEQ ID NO: 8 - DNA, 2422 bp, Sus scrofa
agagatagggtagaggaaagggtctagaatatgtacacgcagctgactcaggcaggctccacgctgaacggttacacagagaggaaacaataaatctcagctactatgcaataaatatctcaagttttaactaaggaaactatcattacagtgaaattaaaaaaataacattttagaacaaagctaacaaatggctagttttctgtgattcttttgcaaaggcgttctttgagagggaaaaagtcaaacaaacaagcagttttacctgaaataaagaactagtttaaaggtcagaagaaaggagcaagttttgcgagaggcacagaacgagagtgctggcaatacaatgacagttttcctttcctttgccttcctcgctgccattctgactcacatagggtgcagcaaccagcgccgaagtccagaaaacggtgggagaagatataaccggattcagcatgggcaatgtgcctacacttttattcttccagaacacgacgggaactgtcgtgagagtacgacagatcagtacaacacaaacgctctgcagagagatgctccacacgtggagcaggatttctcttcccaaaaacttcaacatctggagcatgtgatggaaaattatactcagtggctgcaaaaaattgagaattacattgtggaaaatatgaagtcggagatggcccagatacagcagaatgcagttcagaaccacacggccaccatgcttgagatagggaccagcctcctctctcaaaccgcagagcagaccagaaagctgacagatgttgagacccaggtactaaatcaaacttctcgacttgaaatacaactgctggagaattcattatcaacatacaagctagagaagcaacttcttcaacagacaaatgaaatcctaaagattcatgagaaaaacagtttattagaacataaaatcttagaaatggaggggaagcacaaggaagaattggacaccttaaaggaagagaaagagaatcttcaaggtttggttactcgtcaaacatatataatccaggaactgaaaaagcaattgaacagagccaccaccaataacagtgtccttcaaaagcagcagctggagctgatggacacagtgcataatctcgtcaacctttgcactaaagaaggtgttttactaaagggaggaaaaaaagaggaagtaaaaccatttagagactgtgcagatgtatatcaagctggttttaataaaagtggaatctacactatttatattaataatatgccagaacccaaaaaggtattttgcaatatggatcttaatgggggaggttggactgtaatacaacatcgtgaggatggaagtctagatttccagagaggttggaaagaatataaaatgggttttggaaatccctctggtgaatattggctggggaatgagtttatttttgccataaccagtcagaggcagtacacactgagaaccgagttaatggactgggaaggaaaccgagcctattcacaatacgacagattccacataggaaatgagaagcaaaattacaggttgtatttaaagggtcacagtgggacagcaggaaaacagagcagcctgatcttacatggtgctgatttcagcactaaagatgctgataatgacaactgcatgtgcaaatgtgccctcatgctaacaggaggctggtggtttgatgcttgtggcccatccaatctaaatggaatgttctatacagcgggacagaaccatggaaaactgaatgggataaagtggcattacttcaaagggcccagttactctttacgttccacaactatgatgattcgacctttggacttttgaaagtacaatatcagaagtgatcgtaaaagccacagagaaatctggagaagccaccaaatgagaaattgcgtgaaaacttctgagacaaacagtatcgtcccccttccagcaacaagtagcagttatgtgaaatcaccaaggttcttgactgtgaatgtagaatcttttgagttcacagaagtctctacttggggtgactgtgttcatatggcttgactacagagaatgccactcactgtcatgctttaaaaagggaagaaactgtttagctctctgtgcttcaaactactactggatcttattttggaactttggtagccagatgataaacatggtctatttcatgtaaaacggaagacaaataagaaaataggaacaacaacaaaaaaagagtatacatgaaggataaaaacaggcctgctataattctttggaaaagatgtgttgcaccaataaatggtgttatttctatgcaaacccactaacaataagtttgtactgttgcacaattttaataaaagttcagaaagattaaaaaaaaaaaaaaaaaaaaaaaaaaaaaaaaaaaaaaaaaaaaaa

SEQ ID NO: 9 - DNA, 159 bp, DOG
aggaggaagtctagattttccaagaggttggaaagaatataaaatgggttttggaaatccctctggtgaatattggctggggaatgagtttatttttgccattaccagtcagaggcagtacacactaagaattgagttaatggactgggaaggaaaccc

SEQ ID NO: 10 - DNA, 271 bp, RAT
  modified_base (185)..(244): n = a, c, g, or t/u
aaaaacttgagaattacattgtggaaaatatgaagtcggagatggcccagatacaacagaatgcggttcaaaaccacacggccaccatgctggagataggaaccagcctcttgtctcagactgcagagcagacccgaaagctcacagatgtggagacccaggtactaaatcaaacatcccgtcttgaaatccaactgctggagaattcattatcaacatacaagctagagaaacagcttctccnacagacaaatgaaattctgaagattca

SEQ ID NO: 11 - DNA, 1595 bp, Zebra Fish
tagtttttgatatatttgtgagttttccgtcccatcgggctcctgccatgtggtggggttgcttgtttctggcagcgctgctggtggttgcagattgtggaggagttgagcagaaaacagacggactctggagctccacgccgaaatccaacagcagcagcggtcggaggtatcaccggatccagcatggacagtgcagctacacgtttatcctcccggagagcgatggaaacacttgtcgagagtttaaatcgggcacggcgtacaatgccaatgcattacagcgggacgctcctcaacccgaagccgacttgtccaaccagaagatccagcagctggagcacgtcatggagaactacacgcagtggctacagaagattgagaactacatcaaagacaacatgaagacggagatggtgcagctacaacagagcgccgtccacaaccacacggcagccatgctggaaatgggcaccagtcttctgtcccaaacggcagaacagacgcgaaaactgaccgacgtggagacccaggttttgaatcagacgtcgcggttggaaattcagctgctggagaactcgctgtccaccaataagctggagaagcagctcatgatacagatcaatgaaatcaacaagattcatgacaagaatgggtttctggaggagaagatgcaggagctggaggatcggcaccgtcaggagctggagtctctgcggacggagaaatctgacctgcaggctctggtcagcagacagagttcagtgatccgcgagctggagaatcagctgagcagagctaccggaaacagcacagcacttcagagacagcagcaggacctgatggagagcatgcgcagcctcctcagcctctgtgctaaagacgccgctacagcagtggaaccgaacagcaccaagcaggctgacgaggagcgaaagtttcgggattgtgctgatctttatcaagcaggcttccagaaaaacggagtttacaccatcaatattagcccacaagagaccaaaaaggtgtactgtgtgatggagtctgctggcggaggctggacggtgatccagaagcgtgaagacggcactgtggatttccagaaaacatggaaagaatataagatgggttttggaagcgtgtcgggcgaacactggctggggaacgagtttgtccacgttctgaccaatcagaggcagcacggcctccgcgtggagctgagtgactgggacggacaccaggccttttctcagtacgacagcttccacatcgacagcgagaaacagaaatacaggttgtttttgaagacccacagcgggacggcgggacggcagagcagtttggccgttcatggagcagacttcagcaccaaagacgtggataatgataactgcacatgcaagtgtgcactgatgctcagcggcggctggtggtacgacgcctgcgggccatccaacctgaacggtgtgtattacagacaaggacagcatgtcggaaaatttaatggaatcaaatggcactatttcaaagggcccagttactctctgcgctccaccgtcatgatgatccgaggagccgacttctcatagtcctct

SEQ ID NO: 12 - DNA, 2044 bp, Mus musculus; identical to SEQ ID NO: 2

SEQ ID NO: 13 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 14 - protein, 498 aa, Mus musculus
MTVFLSFAFFAAILTHIGCSNQRRNPENGGRRYNRIQHGQCAYTFILPEHDGNCRESATEQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVSRQTFIIQELEKQLSRATNNNSILQKQQLELMDTVHNLVSLCTKEGVLLKGGKREEEKPFRDCADVYQAGFNKSGIYTIYFNNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 15 - protein, 481 aa, Bos taurus
MTVFLSFAFLAAILTHIGCSNQRRSPENGGRRYNRIQHGQCAYTFILPEHDGNCRESTTDQYNTNALQRDAPHVEQDFSSQKLQHLEHVMENYTQWLQKIENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKIFEMEGKHKEELDTLKEEKENLQGLVTRQTYIIQELEKQLNRATTNNSVLQKQQLELMDTVHNLVNLCTKEVLLKGGKRKEEKPFRDCADVYQAGFNKSGIYTIYINNMPEPKKVFCDMDLNGGGWTVIQHREDGSLDFQRGWKEYKMGFGNPSGEYWLGNEFIFAITSQRQYTLRIELLDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGP

SEQ ID NO: 16 - protein, 110 aa, RAT
ENYTQWLEKLENYIVENMKSEMAQIQQNAVQNHTATMLEIGTSLLSQTAEQTRKLTDVETQVLNQTSRLEIQLLENSLSTYKLEKQLLQQTNEILKIQKKNSLLEHKILE

SEQ ID NO: 17 - protein, 110 aa, Bos taurus
ENYTQWLQKIENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKIFE

SEQ ID NO: 18 - protein, 192 aa, Homo sapiens
MPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 19 - protein, 110 aa, RAT; identical to SEQ ID NO: 16

SEQ ID NO: 20 - protein, 498 aa, Sus scrofa
MTVFLSFAFLAAILTHIGCSNQRRSPENGGRRYNRIQHGQCAYTFILPEHDGNCRESTTDQYNTNALQRDAPHVEQDFSSQKLQHLEHVMENYTQWLQKIENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELKKQLNRATTNNSVLQKQQLELMDTVHNLVNLCTKEGVLLKGGKKEEVKPFRDCADVYQAGFNKSGIYTIYINNMPEPKKVFCNMDLNGGGWTVIQHREDGSLDFQRGWKEYKMGFGNPSGEYWLGNEFIFAITSQRQYTLRTELMDWEGNRAYSQYDRFHIGNEKQNYRLYLKGHSGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 21 - protein, 53 aa, Artificial Sequence
  Description of Artificial Sequence  Synthetic Peptide
GGSLDFPRGWKEYKMGFGNPSGEYWLGNEFIFAITSQRQYTLRIELMDWEGNP

SEQ ID NO: 22 - protein, 89 aa, RAT
  MOD_RES (81): Xaa = anything
KLENYIVENMKSEMAQIQQNAVQNHTATMLEIGTSLLSQTAEQTRKLTDVETQVLNQTSRLEIQLLENSLSTYKLEKQLLXQTNEILKI

SEQ ID NO: 23 - protein, 513 aa, Zebra Fish
MWWGCLFLAALLVVADCGGVEQKTDGLWSSTPKSNSSSGRRYHRIQHGQCSYTFILPESDGNTCREFKSGTAYNANALQRDAPQPEADLSNQKIQQLEHVMENYTQWLQKIENYIKDNMKTEMVQLQQSAVHNHTAAMLEMGTSLLSQTAEQTRKLTDVETQVLNQTSRLEIQLLENSLSTNKLEKQLMIQINEINKIHDKNGFLEEKMQELEDRHRQELESLRTEKSDLQALVSRQSSVIRELENQLSRATGNSTALQRQQQDLMESMRSLLSLCAKDAATAVEPNSTKQADEERKFRDCADLYQAGFQKNGVYTINISPQETKKVYCVMESAGGGWTVIQKREDGTVDFQKTWKEYKMGFGSVSGEHWLGNEFVHVLTNQRQHGLRVELSDWDGHQAFSQYDSFHIDSEKQKYRLFLKTHSGTAGRQSSLAVHGADFSTKDVDNDNCTCKCALMLSGGWWYDACGPSNLNGVYYRQGQHVGKFNGIKWHYFKGPSYSLRSTVMMIRGADFS

SEQ ID NO: 24 - protein, 498 aa, Mus musculus; identical to SEQ ID NO: 14

SEQ ID NO: 25 - protein, 110 aa, RAT; identical to SEQ ID NO: 16

SEQ ID NO: 26 - protein, 498 aa, Mus musculus; identical to SEQ ID NO: 14

SEQ ID NO: 27 - protein, 481 aa, Bos taurus; identical to SEQ ID NO: 15

SEQ ID NO: 28 - protein, 498 aa, Homo sapiens; identical to SEQ ID NO: 13

SEQ ID NO: 29 - DNA, 27 bp, Artificial Sequence
  Description of Artificial Sequence  Synthetic Primer
cggtcggtcttcgaaacgtttacccta

SEQ ID NO: 30 - DNA, 27 bp, Artificial Sequence
  Description of Artificial Sequence  Synthetic Primer
tgtgaagcgtctcacaggtccaggatg

SEQ ID NO: 31 - DNA, 20 bp, Artificial Sequence
  Description of Artificial Sequence  Synthetic Primer
taatgtcaggttggaggggg

SEQ ID NO: 32 - DNA, 20 bp, Artificial Sequence
  Description of Artificial Sequence  Synthetic Primer
agggcacatttgcacataca

SEQ ID NO: 33 - DNA, 20 bp, Artificial Sequence
  Description of Artificial Sequence  Synthetic Primer
caaggagagaggggtctagg

SEQ ID NO: 34 - DNA, 20 bp, Artificial Sequence
  Description of Artificial Sequence  Synthetic Primer
ctctgcaccgagtcatcgta

SEQ ID NO: 35 - protein, 162 aa, Mus musculus
MHFSERDHACAFSEITEHGALHSLIPYPGGTETHALAFPYNDSKPEWCSNQRRNPENGGRRYNRIQHGQCAYTFILPEHDGNCRESATEQYNTNALQRDAPHVEPDFSSQKLQHLEHVMENYTQWLQKGAQSSVGGICVYLLLRGAVFAKEKRLPLWTQLPG

SEQ ID NO: 36 - protein, 402 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYR

SEQ ID NO: 37 - protein, 497 aa, RAT
MTVFLSFAFFAAILTHIGCSNQRRSPENGGRRYNRIQHGQCAYTFILPEHDGNCRESATEQYNTNALQRDAPHVETDFSSQKLQHLEHVMENYTQWLQKLENYIVENMKSEMAQIQQNAVQNHTATMLEIGTSLLSQTAEQTRKLTDVETQVLNQTSRLEIQLLENSLSTYELEKQLLQQTNEILKIQEKNSLLEHKILEMEGKHKEELDTLKEEKENLQGLVTRQTFIIQELEKQLSRATSNNSVLQKQQLELMDTVHNLVSLCTKEVLLKGGKREEEKPFRDCADVYQAGFNKSGIYTIYFNNMPEPKKVFCNMDVNEGGWTVIQHREDGSLDFQRGWKEYKMGFGNPSGEYWLGNEFIFAITSQRQYMLRIELMDWEGNRAYSQYDRFHIGNQKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 38 - protein, 497 aa, RAT; identical to SEQ ID NO: 37

SEQ ID NO: 39 - protein, 497 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 40 - protein, 498 aa, Homo sapiens; identical to SEQ ID NO: 13

SEQ ID NO: 41 - protein, 147 aa, Homo sapiens
MDTVHNLVNLCTKEVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYR

SEQ ID NO: 42 - protein, 497 aa, Homo sapiens; identical to SEQ ID NO: 39

SEQ ID NO: 43 - protein, 513 aa, Zebra Fish; identical to SEQ ID NO: 23

SEQ ID NO: 44 - DNA, 2066 bp, Homo sapiens
atttctccatgtggcagacagagcaaagccacaacgctttctctgctggattaaagacggcccacagaccagaacttccactatactacttaaaattacataggtggcttgtcaaattcaattgattagtattgtaaaaggaaaaagaagttccttcttacagcttggattcaacggtccaaaacaaaaatgcagctgccattaaagtcacagatgaacaaacttctacactgatttttaaaatcaagaataagggcagcaagtttctggattcactgaatcaacagacacaaaaagctggcaatatagcaactatgaagagaaaagctactaataaaattaacccaacgcatagaagacttttttttctcttctaaaaacaactaagtaaagacttaaatttaaacacatcattttacaacctcatttcaaaatgaagacttttacctggaccctaggtgtgctattcttcctactagtggacactggacattgcagaggtggacaattcaaaattaaaaaaataaaccagagaagataccctcgtgccacagatggtaaagaggaagcaaagaaatgtgcatacacattcctggtacctgaacaaagaataacagggccaatctgtgtcaacaccaaggggcaagatgcaagtaccattaaagacatgatcaccaggatggaccttgaaaacctgaaggatgtgctctccaggcagaagcgggagatagatgttctgcaactggtggtggatgtagatggaaacattgtgaatgaggtaaagctgctgagaaaggaaagccgtaacatgaactctcgtgttactcaactctatatgcaattattacatgagattatccgtaagagggataattcacttgaactttcccaactggaaaacaaaatcctcaatgtcaccacagaaatgttgaagatggcaacaagatacagggaactagaggtgaaatacgcttccttgactgatcttgtcaataaccaatctgtgatgatcactttgttggaagaacagtgcttgaggatattttcccgacaagacacccatgtgtctcccccacttgtccaggtggtgccacaacatattcctaacagccaacagtatactcctggtctgctgggaggtaacgagattcagagggatccaggttatcccagagatttaatgccaccacctgatctggcaacttctcccaccaaaagccctttcaagataccaccggtaactttcatcaatgaaggaccattcaaagactgtcagcaagcaaaagaagctgggcattcggtcagtgggatttatatgattaaacctgaaaacagcaatggaccaatgcagttatggtgtgaaaacagtttggaccctgggggttggactgttattcagaaaagaacagacggctctgtcaacttcttcagaaattgggaaaattataagaaagggtttggaaacattgacggagaatactggcttggactggaaaatatctatatgcttagcaatcaagataattacaagttattgattgaattagaagactggagtgataaaaaagtctatgcagaatacagcagctttcgtctggaacctgaaagtgaattctatagactgcgcctgggaacttaccagggaaatgcaggggattctatgatgtggcataatggtaaacaattcaccacactggacagagataaagatatgtatgcaggaaactgcgcccactttcataaaggaggctggtggtacaatgcctgtgcacattctaacctaaatggagtatggtacagaggaggccattacagaagcaagcaccaagatggaattttctgggccgaatacagaggcgggtcatactccttaagagcagttcagatgatgatcaagcctattgactgaagagagacactcgccaatttaaatgacacagaactttgtacttttcagctcttaaaaatgtaaatgttacatgtatattacttggcacaatttatttctacacataaagtttttaaaatgaattttaccgtaactataaaagggaacctataaatgt

SEQ ID NO: 45 - DNA, 1891 bp, RAT
aggctccacgctgaacggttacacagagaggaaacaataaatctaagctactattgcaataaatatctcaagttttaacgaaggaaactatcattacagtaatttttttaagtaacgcgttttataacaaagctaacaaatggctagttttctgtggatcttcttcaaatgctttctttaacggggagagcgtcaaacaaccagttttacctgaaataaagaactagtttaaaggtcagaagagaggagcaagctttgcaggaggcacggaaggcgagtgctggcagtacaatgacagttttcctttcctttgcattcttcgctgccattctgactcacatagggtgcagcaaccagcgccggagtccagaaaacggagggagaagatataaccgaattcaacatgggcaatgtgcctacactttcattcttccagaacacgacgggaactgccgtgagagtgcgacagagcagtacaacaccaacgctctgcaaagggatgctccacacgtggagacggatttctcttcccagaaacttcagcatctggagcatgtgatggaaaattatactcagtggctgcaaaaacttgagaattacattgtggaaaatatgaagtcggagatggcccagatacaacagaatgcggttcaaaaccacacggccaccatgctggagataggaaccagcctcttgtctcagactgcagagcagacccgaaagctcacagatgtggagacccaggtactaaatcaaacatcccgtcttgaaatccaactgctggagaattcattatcaacatacgagctagagaaacagcttctccaacagacaaatgaaattctgaagattcaggaaaaaaacagtttattagagcataaaatcctagaaatggagggaaaacacaaggaagagctggacaccttgaaggaggagaaagaaaaccttcaaggcttggttactcgtcagacattcatcatccaagaattggagaagcaacttagcagagctaccagcaacaacagtgttctgcagaagcaacaactggagctcatggacacagtccataaccttgtcagcctttgcacaaaagaagttttgctaaagggaggaaaaagagaagaagagaaaccatttcgagactgtgcagatgtatatcaagctggttttaataagagtggaatctacactatttattttaataatatgccagaacccaaaaaggtattttgcaatatggatgtgaatgaaggaggatggacagtaatacaacaccgtgaggatggaagcctagatttccagaggggctggaaggagtataaaatgggttttgggaatccctctggtgaatattggcttgggaacgagttcatttttgcaataaccagtcagaggcagtacatgctgaggatcgagctgatggactgggaagggaaccgagcctactcacagtacgacagattccacataggaaaccagaagcagaactacaggttatatttaaagggtcacacggggacagcaggcaaacagagcagcttgatcttacatggtgctgatttcagcacaaaggacgctgataacgacaactgtatgtgcaaatgcgcccttatgctaacaggaggttggtggtttgatgcctgtggcccttccaatctaaacggaatgttctacactgcagggcaaaaccatggaaaactgaatgggataaagtggcactacttcaaaggacccagttactccttacgttccacaaccatgatgatccggcccttggacttctgaaggcgctatgcctagtattagaaacctgaaataaatctggggatgttcccgaatgagaagctatctggaagcttccgaaacaacccagcattgtctccgttccagc

SEQ ID NO: 46 - DNA, 2379 bp, Homo sapiens
ggggcacactcatgcattcctgtcaagtcatcttgtgaaaggctgcctgcttccagcttggcttggatgtgcaaccttaataaaactcactgaggtctgggagaaaatagcagatctgcagcagatagggtagaggaaagggtctagaatatgtacacgcagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtacaatgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggtaagtcatgctacagtgtagtgttcgactaccttttacctagccacttaataattgtagtgggaaaatatgaaaaaaagaaatgaaaatcatccttcaaaatgaaaattcttttttttttgtctagacctattacaaagaggtacaaaaaagcctgtaaagactttttcaaagatttaaatttcagttttcgtaggttgactatctttgatattggcttaaattttagggaatggtgaatgaagtcaagcatactgttgttctgtggttatcatgcagttgcagtgaattcatatatataaaactgataagtttcatactcaaactcattggcttaaataatctctttattctaaaaaaagattttctggttctactttttttgatgacctttcttgaattagcatttttcaaatgatcttgaagatgacaaaagtaaaatttttttatgtttgaatgatttacataaaagagaagatgaggcatgatttagagggttttcttggtaaatctaaaaagcaaaaataaattgtaataatggcatcaatatacataatacatgattgcagatgtaaaaatagataaactatacacttaatgatacatgtttccaattatcctttttcaatgagaagctcgtctactagggaaaaataaacacacaaaaatatggctaaaaaaaaaaaaaaaaaaaaaaaaaaaaaaa

SEQ ID NO: 47 - DNA, 489 bp, Mus musculus
atgcatttcagtgaacgggatcatgcttgtgcattctctgagatcactgaacatggagccttacacagcctgataccatatcccggtgggactgagactcatgctttagcttttccatataatgacagtaaaccagaatggtgcagcaaccagcgccgaaatccagaaaacggagggagaagatataaccggattcaacatgggcaatgtgcctacactttcattcttccagaacacgacgggaactgccgtgagagtgcgacagagcagtacaacaccaacgctctgcaaagggatgctccacacgtggagccggatttctcttcccagaaacttcagcatctggagcatgtgatggaaaattatactcagtggctgcaaaaaggagctcagagttccgtagggggcatttgtgtttaccttttgctccggggggcagtgtttgcaaaagaaaaaaggctccctctctggactcaacttcctgggtaa

SEQ ID NO: 48 - DNA, 1891 bp, RAT; identical to SEQ ID NO: 45

SEQ ID NO: 49 - DNA, 1494 bp, Homo sapiens
atgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaagttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 50 - DNA, 1497 bp, Homo sapiens
atgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 51 - DNA, 1201 bp, Homo sapiens
aaaacagagctaccaccaacaacagtgtccttcagaagcagcaactggagctgatggacacagtccacaaccttgtcaatctttgcactaaagaagttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggtaagtcatgctacagtgtagtgttcgactaccttttacctagccacttaataattgtagtgggaaaatatgaaaaaaagaaatgaaaatcatccttcaaaatgaaaattcttttttttttgtctagacctattacaaagaggtacaaaaaagcctgtaaagactttttcaaagatttaaatttcagttttcgtaggttgactatctttgatattggcttaaattttagggaatggtgaatgaagtcaagcatactgttgttctgtggttatcatgcagttgcagtgaattcatatatataaaactgataagagtttcatactcaaactcattggcttaaataatctctttattctaaaaaaagattttctggttctactttttttgatgacctttcttgaattagcatttttcaaatgatcttgaagatgacaaaagtaaaatttttttatgtttgaatgatttacataaaagagaagatgaggcatgatttagagggttttcttggtaaatctaaaaagcaaaaataaattgtaataatggcatcaatatacataatacatgattgcagatgtaaaaatagataaactatacacttaatgatacatgtttccaattatcctttttcaatgagaagctcgtctactagggaaaaataaacacacaaaaatatggctaaaaaaaaaaaaaaaaaaaaaaaaaaaaaaa

SEQ ID NO: 52 - DNA, 1494 bp, Homo sapiens; identical to SEQ ID NO: 49

SEQ ID NO: 53 - DNA, 1972 bp, Homo sapiens
atccgtgatcttaggtttgttggaatgattaagtgagatagtgtgtaaggagtgcttcgcctggaaaacagtaaggtcttaatgcatgccattattattgttgcacaggagtgatacatgactcaatgaggagacacgcaaaagttcaagaggctgcaaaagtggttcaaatattttattgctgtgtttggcataacttgcttccatatctgtccaatcagaaatttgttatttggtgtggaaaggactgatgaatctcggcaaggtttctttccccaagtaaatcgcatttgttctggaatttgttttgacctctcctctaacttctccctaaatgcccatctctaaccactacggaccttcacccactgccctgttctccttccaagacatgggagatgacagctgtggcttctgttttcacaccatgactgcagttgtctaaaagccctcccagttcctaatgaaaatcccaactcataattcaggttttctgcatctagtaaagatctcaaagccgttccctaaattctacaattattgcctgtttatgtttatgggtccctttgttcaaatgtttatacgctgcctgtgggaaatcatcttctgataatttttctttcttccttcacagaagcacagagatgtctcctgtacagcccattgctggctgttagcacacctgatatatgcttgttctcacatagtatgtctgtgcagccacaggaggtttttatgtggaacttccacaccagtgttaaagacagccatagccaccaaagagatccttgtaaaatacaggattaggatataccacaggaattaataattctcaagccactgctttaaattgtagccacattgtttttgtagggaagttgctcaatttatatacacagaacatgaagggttgcattctgatcaggacaccagtttcatttaagaaccctataccaaaaggtacttagataaccctctttaatatgcacatactatattgctttgtgcaacatattgctttgagcaacaaatgatgctaattttcttgctaatttgatcctctctgattattttcttcaactcctaaaagtctaagtgaatacaatgaaagaataccatctgtaaaaactgttttttaacatatttttcctcagagtcaagaatacatgttttttaaagacatttaataatgatatgggtttatcattctctatcaaaaataattccattggacattatgccaattttcttcttagggtccctcgactaaatagtaactttaaaaacatactgctagtttgctatattttagtaggtcagcatgcacaccaaaagtgtattaaggtggactgctctatttattgatttgccatgaatctgctaaaggctcaaggcaattgtctgtggaaaggggcgggagggccatacatgatcgaggtcatgcgcatctagccaatttgtaagacgatcccgccgccctaagccatcagcaatccttagtataggggcacactcatgcattcctgtcaagtcatcttgtgaaaggctgcctgcttccagcttggcttggatgtgcaaccttaataaaactcactgaggtctgggagaaaatagcagatctgcagcagatagggtagaggaaagggtctagaatatgtacacgcagctgactcaggcaggctccatgctgaacggtcacacagagaggaaacaataaatctcagctactatgcaataaatatctcaagttttaacgaagaaaaacatcattgcagtgaaataaaaaattttaaaattttagaacaaagctaacaaatggctagttttctatgattcttcttcaaacgctttctttgagggggaaagagtcaaacaaacaagcagttttacctgaaataaagaactagttttagaggtcagaagaaaggagcaagttttgcgagaggcacggaaggagtgtgctggcagtaca

SEQ ID NO: 54 - DNA, 1595 bp, Zebra Fish; identical to SEQ ID NO: 11

SEQ ID NO: 55 - protein, 8 aa, Homo sapiens
FSSQKLQH

SEQ ID NO: 56 - protein, 8 aa, Homo sapiens
FRDCADVY

(19) United States
(12) Patent Application Publication
(10) Pub. No.: US 2003/0220250 A1
(43) Pub. Date: Nov. 27, 2003

(54) Angiopoietin-1 in the treatment of disease

(76) Inventor: Lee M. Ellis, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/367,259

(22) Filed: Feb. 14, 2003

Related U.S. Application Data
(60) Provisional application No. 60/356,809, filed on Feb. 14, 2002.

(51) Int. Cl. A61K 38/17; A61K 48/00

(52) U.S. Cl. 514/12; 514/44

(57) ABSTRACT

The present invention regards angiopoietin-1, which is a stabilizing factor for endothelial cells. The invention relates to angiopoietin-1 specifically as an inhibitor of tumor growth and angiogenesis, particularly for the treatment of cancer. In a particular embodiment, angiopoietin-1 is utilized to induce tumor dormancy or limit tumor growth by stabilizing the endothelium and preventing endothelial cell proliferation, and, thus, preventing angiogenesis.

The present invention claims priority to U.S. Provisional Patent

Application Serial No. 60/356,809, filed Feb. 14, 2002, incorporated by reference herein in its entirety.

### FIELD OF THE INVENTION

The present invention is directed to methods and compositions for the treatment of disease using angiopoietin-1. In specific embodiments, angiopoietin-1 is used to inhibit angiogenesis, particularly in the treatment of cancer.

### BACKGROUND OF THE INVENTION

Angiogenesis is the development of new blood vessels from existing microvessels. The process of generating new blood vessels plays an important role in embryonic development, in the inflammatory response, in the development of metastases (tumor-induced angiogenesis), in diabetic retinopathy, in the formation of the arthritic panus, in psoriasis, and so forth. Under normal physiological conditions, humans or animals only undergo angiogenesis in very specific, restricted situations. For example, angiogenesis is normally observed in wound healing, in fetal and embryonal development, and in the formation of the corpus luteum, endometrium and placenta. The control of angiogenesis is a highly regulated system involving angiogenic stimulators and inhibitors. The control of angiogenesis has been found to be altered in certain disease states and, in many cases, the pathological damage associated with the disease is related to the uncontrolled angiogenesis.

In tumor angiogenesis, for example, capillary sprouts are formed, their formation being induced by a group of tumor cells. However, compared with blood vessels produced in normal angiogenic microenvironments, tumor microvessels are morphologically and functionally unique. Their vascular networks typically show disorganized or aberrant architecture, luminal sizes vary and blood flow can fluctuate chaotically. There are two principal types of tumor angiogenesis in terms of the events that follow implantation of metastatic seedlings on surfaces and in organs. The first or primary angiogenesis is the initial vascularization of the mass of multiplying tumor cells and is regarded as an essential prerequisite for the survival and further growth of a metastatic deposit. The second is a continuing or secondary angiogenesis and is the phenomenon that occurs in waves at the periphery of a growing tumor mass. This second angiogenesis is essential for the accretion of new microcirculatory territories into the service of the expanding and infiltrating tumor.

Persistent, unregulated angiogenesis occurs in a multiplicity of disease states, tumor metastasis and abnormal growth by endothelial cells and supports the pathological damage seen in these conditions. The diverse pathological states created due to unregulated angiogenesis have been grouped together as angiogenic-dependent or angiogenic-associated diseases. Therapies directed to the control of the angiogenic processes could lead to the abrogation or mitigation of these diseases.

One example of a disease mediated by angiogenesis is ocular neovascular disease. This disease is characterized by invasion of new blood vessels into the structures of the eye, such as the retina or cornea. It is the most common cause of blindness and is involved in approximately twenty eye diseases. In age related macular degeneration, the associated visual problems are caused by an ingrowth of choroidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia. Diseases associated with retinal/choroidal neovascularization include, but are not limited to, diabetic retinopathy, macular degeneration, sickle cell anemia, sarcoid, syphilis, pseudoxanthoma elasticum and Pagets disease.

Another disease in which angiogenesis is believed to be involved is rheumatoid arthritis. The blood vessels in the synovial lining of the joints undergo angiogenesis. In addition to forming new vascular networks, the endothelial cells release factors and reactive oxygen species that lead to pannus growth and cartilage destruction.

Among the angiogenic factors involved in angiogenesis, angiopoietins are novel endothelial growth factors that are ligands for the endothelium-specific tyrosine kinase receptor Tie-2 (Peters, 1998). Of the four currently known angiopoietins (Ang-1 to Ang-4), the best characterized are Ang-1 and Ang-2. Ang-1 binds to the Tie-2 receptor and activates it by inducing phosphorylation and dimerization of the known domains. Ang-2 also binds to Tie-2 but does not induce phosphorylation and antagonizes the action of Ang-1. Vascular endothelial growth factor (VEGF) and the angiopoietins seem to play complementary and coordinated roles in the development of new blood vessels. Ang-1 helps to maintain and stabilize mature vessels by promoting interaction between endothelial cells and supporting cells (Maisonpierre et al., 1997; Suri et al., 1998; Papapetropoulous et al., 1999). Ang-2 is expressed at sites of vascular remodeling (Maisonpierre et al., 1997) and is thought to block the stabilizing action of Ang-1. Destabilization by Ang-2 in the presence of VEGF has been hypothesized to induce an angiogenic response; however, in the absence of VEGF, Ang-2 leads to vessel regression (Maisonpierre et al., 1997; Suri et al., 1997; Ssato et al., 1995; Dumont et al., 1994).

U.S. Pat. Nos. 5,521,073 and 5,643,755 are directed to nucleic acid molecules encoding human TIE-2 ligand and methods of making them. U.S. Pat. Nos. 5,650,490 and 5,879,672 regard human TIE-2 ligands.

Hayes et al. (2000) demonstrate that overexpression of Ang1 in MCF-7 breast cancer cells inhibits their growth in mice.

Wong et al. (2000) show Ang1 expression decreases while Ang2 expression increases in non-small cell lung cancers compared to normal lung tissue.

Shim et al. (2001) show inhibition of human cervical carcinoma HeLa cells following inhibition of Ang1 expression by an antisense RNA approach.

Thus, although most of the research on angiopoietins has focused on vasculogenesis (Peters, 1998; Maisonpierre et al., 1997; Papapetropoulos et al., 1999; Asahara et al., 1998; Witzenbichler et al., 1998; Kwak et al., 1999); few reports have focused on tumor angiogenesis (Stratman et al., 1998; Tanaka et al., 1999; Zagzag et al., 1999), and none have investigated the role of angiopoietins in human colon cancer.

### BRIEF SUMMARY OF THE INVENTION

The present invention is directed to a system and method which are related to treatment with angiopoietin-1 of tumors and other medical conditions related to angiogenesis.

Angiopoietin-1 (Ang-1) and angiopoietin-2 (Ang-2) are important regulators of endothelial cell (EC) survival. Current models suggest that an increase in Ang-2 expression in ECs leads to initiation of angiogenesis. The present invention shows that HT29 colon cancer cells were stably transfected with cDNA constructs for Ang-1 or Ang-2 or with vector alone, the cells were injected subcutaneously into nude mice, and tumor growth was assessed. Immunohistochemical analyses confirmed sustained increases of Ang-1 and Ang-2 in the tumors. The tumors produced by the Ang-2-transfected cells were larger than the tumors produced in the other groups; those tumors also had higher vessel counts and proliferative indices than tumors in the other groups. Tumors produced by the Ang-1 transfectants had fewer vessels and lower tumor cell proliferative indices than tumors in the other groups. Furthermore, Ang-2 is expressed in colon cancer epithelium whereas Ang-1 is not. This imbalance favoring Ang-2 activity, in a specific embodiment, induces blood vessel destabilization and initiate angiogenesis in colon cancer.

Moreover, the present invention teaches that overexpression of Ang-1 reduces tumor growth and angiogenesis of colon cancer hepatic metastases and furthermore prevents ascites formation in a peritoneal carcinomatosis model of colorectal cancer by reducing vascular permeability. A skilled artisan recognizes it can be used for liver tumors, as the endothelium from liver, in specific embodiments, is the same for metastasis from various sites as well as for primary liver tumors and also for peritoneal carcinomatosis, such as ovarian cancer and pancreatic cancer. Some examples of peritoneal surface malignancies include appendix cancer and pseudomyxoma peritonei, colon cancer with peritoneal carcinomatosis, gastric cancer with peritoneal carcinomatosis, abdominopelvic sarcoma with sarcomatosis, and primary peritoneal surface malignancy including peritoneal mesothelioma, papillary serous cancer, and primary peritoneal adenocarcinoma.

In specific embodiments of the present invention, Ang-1 is utilized as an anti-neoplastic therapy where tumor dormancy is induced or tumor growth is limited by stabilizing the endothelium and preventing endothelial cell proliferation, and, thus, preventing angiogenesis.

In specific embodiments of the present invention, an angiopoietin-1 polypeptide may be contacted with or introduced to a cell through any of a variety of manners known to those of skill in the art. The angiopoietin-1 polypeptide may be introduced through direct introduction of an angiopoietin-1 polypeptide to a cell. In this case, the angiopoietin-1 polypeptide may be obtained through any method known in the art, although it is expected that in vitro expression of the angiopoietin-1 polypeptide in a cell culture system may be a preferred manner of obtaining angiopoietin-1.

Angiopoietin-1 may also be introduced to a cell via the introduction of a polynucleotide that encodes the angiopoietin-1 polypeptide to the cell. For example, RNA or DNA encoding angiopoietin-1 may be introduced to the cell by any manner known in the art. In certain preferred embodiments, the angiopoietin-1 is introduced into the cell through the introduction of a DNA segment which encodes angiopoietin-1. In some such embodiments, it is envisioned that the DNA segment further comprises the angiopoietin-1 gene operatively linked to its associated control sequences. For example, the angiopoietin-1 gene may be operatively linked to a suitable promoter and a suitable terminator sequence. The construction of such gene/control sequence DNA constructs is well-known within the art. In particular embodiments the promoter is selected from the group comprising of CMV IE, SV40 TE, RSV LTR, or β-actin. In certain embodiments for introduction, the DNA segment may be located on a vector, for example, a plasmid vector or a viral vector. A liposome, in other specific embodiments, is utilized to transfer the angiopoietin-1 to the tumor. The virus vector may be, for example, selected from the group comprising retrovirus, adenovirus, herpesvirus, vaccina virus, and adeno-associated virus. Such a DNA segment may be used in a variety of methods related to the invention. The vector may be used to deliver an angiopoietin-1 gene to a cell in one of the gene-therapy embodiments of the invention. Also, such vectors can be used to transform cultured cells, and such cultured cells could be used, inter alia, for the expression of angiopoietin-1 in vitro.

In particular embodiments the angiopoietin-1 is introduced into a cell that is a human cell. In many embodiments the cell is a tumor cell. In some presently preferred embodiments the tumor cell is a colon tumor cell. In some embodiments, the angiopoietin-1 is introduced by injection.

In some embodiments of the present invention, the inventor's discovery that angiopoietin-1 is useful for treating cancer will be used in combination with other anti-cancer therapies. These other therapies may be known at the time of this application, or may become apparent after the date of this application. Angiopoietin-1 may be used in combination with other therapeutic polypeptides, polynucleotides encoding other therapeutic polypeptides, or chemotherapeutic agents. For example, angiopoietin-1 may be used in conjunction with other known polypeptides, such as TNFα or p53. Angiopoietin-1 may be used in conjunction with any suitable chemotherapeutic agent. Angiopoietin-1 also may be used in conjunction with radiotherapy. The type of ionizing radiation constituting the radiotherapy may be selected from the group comprising x-rays, y-rays, and microwaves. In certain embodiments, the ionizing radiation may be delivered by external beam irradiation or by administration of a radionuclide. Angiopoietin-1 also may be used with other gene-therapy regimes. In particular embodiments, the angiopoietin-1 is introduced into a tumor. The tumor may be in an animal, in particular, a human. The angiopoietin-1 may be introduced by injection.

Another aspect of the present invention is a method for inhibiting tumor cell proliferation, the method comprising contacting a tumor cell with an angiopoietin-1 polypeptide in an amount effective to inhibit tumor cell proliferation. In representative embodiments of the invention, angiopoietin-1 is introduced to the tumor cell through direct introduction of an angiopoietin-1 polypeptide or through the introduction of a polynucleotide encoding an angiopoietin-1 polypeptide.

Another aspect of the present invention, is a method for altering the phenotype of a tumor cell comprising contacting a tumor cell with an angiopoietin-1 polypeptide in an amount effective to alter the phenotype of the tumor cell. The phenotype of the tumor cell may be selected from a group comprising, but not limited to, proliferation, soft agar growth, migration, contact inhibition or cell cycling.

The angiopoietin-1 gene products and polynucleotides of the present invention may also be introduced using any suitable method. A “suitable method” of introduction is one that places an angiopoietin-1 gene product in a position to reduce the proliferation of a tumor cell, alter the phenotype of a tumor cell, and/or inhibit the transformation of a cell. For example, injection, oral, and inhalation methods may be employed, with the skilled artisan being able to determine an appropriate method of introduction for a given circumstance. In some preferred embodiments, injection will be used. This injection may be intravenous, intraperitoneal, intramuscular, subcutaneous, intratumoral, intrapleural, or of any other appropriate form.

In certain other aspects of the present invention there are provided therapeutic kits comprising in a suitable container, a pharmaceutical formulation of an angiopoietin-1 gene product or a polynucleotide encoding an angiopoietin-1 gene product. Such a kit may further comprise a pharmaceutical formulation of a therapeutic polypeptide, polynucleotide encoding a therapeutic polypeptide, or chemotherapeutic agent.

In one embodiment of the present invention, there is a method of stabilizing the endothelium and preventing endothelial cell proliferation associated with a tumor, the method comprising contacting the tumor with an angiopoietin-1 polypeptide in an amount effective to prevent angiogenesis of the tumor. The term “associated with” comprises embodiments wherein the endothelium and/or the endothelial cells are in the tumor, surrouding the tumor, adjacent to the tumor, or a combination thereof. In specific embodiments, the angiopoietin-1 polypeptide is introduced to the tumor through the introduction of an angiopoietin-1-encoding polynucleotide. In specific embodiments, the tumor is a colon tumor, a colorectal tumor, a liver tumor, or a peritoneal carcinomatosis. In further specific embodiments, the peritoneal carcinomatosis is appendix cancer, pseudomyxoma peritonei, colon cancer with peritoneal carcinomatosis, gastric cancer with peritoneal carcinomatosis, abdominopelvic sarcoma with sarcomatosis, or a primary peritoneal surface malignancy. In another further specific embodiment, the primary peritoneal surface malignancy is peritoneal mesothelioma, papillary serous cancer, or primary peritoneal adenocarcinoma. In a specific embodiment, the tumor is in a patient. In another specific embodiment, the angiopoietin-1 polypeptide is contacted with the tumor by injection into the tumor. In a further specific embodiment, the contacting step is further defined as injecting a polynucleotide encoding the angiopoietin-1 polypeptide into the patient. In an additional specific embodiment, the injection is orthotopic, intradermal, subcutaneous, intramuscular, intraperitoneal, or intravenous. In another specific embodiment, the injection is regional to the tumor.

In specific embodiments, the method further comprises treating the tumor with a second agent, wherein the second agent is a therapeutic polypeptide, polynucleotide encoding a therapeutic polypeptide, chemotherapeutic agent, or radiotherapeutic agent. In a specific embodiment, the angiopoietin-1 polypeptide is administered by injection. In another specific embodiment, the polynucleotide is a deoxyribonucleic acid molecule that encodes an angiopoietin-1 polypeptide. In an additional specific embodiment, the angiopoietin-1-encoding polynucleotide further comprises control sequences operatively linked to the angiopoietin-1 encoding polynucleotide. In one specific embodiment, the angiopoietin-1-encoding polynucleotide is located on a vector. In another specific embodiment, the polynucleotide is operably linked to a promoter. In an additional specific embodiment, the promoter is selected from the group consisting of CMV IE, SV40 IE, RSV LTR, or α-actin. In one specific embodiment, the vector comprises a plasmid vector. In an additional specific embodiment, the vector comprises a viral vector, such as a retrovirus, adenovirus, herpesvirus, vaccinia virus, or adeno-associated virus.

In another embodiment of the present invention, there is a method of inhibiting angiogenesis related to a disease in an individual, comprising the steps of contacting a cell affected by the disease with an angiopoietin-1 polypeptide in an amount effective to inhibit the angiogenesis. The term “angiogenesis related to a disease” in some embodiments refers to angiogenesis that is the indirect or direct cause of the disease or the indirect or direct result of the disease. In a specific embodiment, the disease is cancer. In a further specific embodiment, the angiopoietin-1 polypeptide is introduced into a cancer cell by the direct introduction of the angiopoietin-1 polypeptide. In an additional specific embodiment, the angiopoietin-1 polypeptide is introduced into the cell through the introduction of an angiopoietin-1-encoding polynucleotide. In another specific embodiment, the cancer is colon cancer, liver cancer, colorectal cancer, or peritoneal adenocarcinoma.

In other embodiments, the method further comprises treating the cancer cell with a second agent, wherein the second agent is a therapeutic polypeptide, polynucleotide encoding a therapeutic polypeptide, chemotherapeutic agent, or radiotherapeutic agent. In a specific embodiment, the angiopoietin-1 polypeptide is administered by injection. In another specific embodiment, the angiopoietin-1 polynucleotide is administered to the individual by injection, such as orthotopic, intradermal, subcutaneous, intramuscular, intraperitoneal, or intravenous. In a further specific embodiment, the polynucleotide is administered with a liposome. In a specific embodiment, the polynucleotide is a deoxyribonucleic acid molecule that encodes an angiopoietin-1 polypeptide. In another specific embodiment, the angiopoietin-1-encoding polynucleotide further comprises control sequences operatively linked to the angiopoietin-1 encoding polynucleotide. In a further specific embodiment, the angiopoietin-1-encoding polynucleotide is located on a vector. In an additional specific embodiment, polynucleotide is operably linked to a promoter, such as CMV IE, SV40 IE, RSV LTR, or α-actin. In a specific embodiment, the vector comprises a plasmid vector or a viral vector, such as retrovirus, adenovirus, herpesvirus, vaccinia virus, or adeno-associated virus.

In another embodiment of the present invention, there is a method of inhibiting growth in a tumor, the method comprising contacting the tumor with an angiopoietin-1 polypeptide in an amount effective to inhibit the growth, wherein the tumor is a colon tumor, a colorectal tumor, or a liver tumor. In a specific embodiment, the angiopoietin-1 polypeptide is introduced into the cell by the direct introduction of the angiopoietin-1 polypeptide. In a specific embodiment, the angiopoietin-1 polypeptide is introduced into the cell through the introduction of an angiopoietin-1-encoding polynucleotide. In another specific embodiment, the colon tumor or colorectal tumor is in a patient.

In an additional embodiment, the method further comprises treating the tumor with a second agent, wherein the second agent is a therapeutic polypeptide, polynucleotide encoding a therapeutic polypeptide, chemotherapeutic agent, or radiotherapeutic agent. In a specific embodiment, the angiopoietin-1 polypeptide is administered by injection into the patient. In another specific embodiment, the polynucleotide is a deoxyribonucleic acid molecule that encodes an angiopoietin-1 polypeptide. In a further specific embodiment, the angiopoietin-1-encoding polynucleotide further comprises control sequences operatively linked to the angiopoietin-1 encoding polynucleotide. In an additional specific embodiment, the angiopoietin-1-encoding polynucleotide is located on a vector. In a further specific embodiment, the polynucleotide is operably linked to a promoter, such as CMV IE, SV40 IE, RSV LTR, or α-actin. In a specific embodiment, the vector comprises a plasmid vector or a viral vector, such as a retrovirus, adenovirus, herpesvirus, vaccinia virus, or adeno-associated virus.

Thus, the present invention is directed to Ang-1 as an important regulator of angiogenesis and vascular permeability. In specific embodiments, Ang-1 increases periendothelial support and vessel stabilization. Therefore, in specific embodiments, Ang-1 is an anti-neoplastic and/or anti-permeability agent for patients with cancer, for example, metastatic colorectal cancer.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

In keeping with long-standing patent law convention, the words “a,” and “an” when used in the present specification in concert with the word comprising, including the claims, denote “one or more.”

### BRIEF DESCRIPTION OF THE DRAWINGS

For a more complete understanding of the present invention, reference is now made to the following descriptions taken in conjunction with the accompanying drawing, in which:

FIG. 1 demonstrates the effect of Ang-2 overexpression on volume and weight of tumors formed by subcutaneous injection of HT29 human colon cancer cells in nude mice. Cells stably transfected with Ang-2 produced significantly larger (A) and heavier (B) tumors than did cells transfected with Ang-1 or pcDNA3.1 vector alone or parental HT29 cells (mean values ±SE). Neither tumor volume nor tumor weight differed among the mice injected with Ang-1-transfected cells, with pcDNA mock-transfected cells, or with parental HT29 cells. *P<0.05 versus other groups (student's t-test).

FIG. 2. shows the effect of Ang-1 and Ang-2 transfection on tumor vessel count and proliferative index (PCNA) in tumors grown in nude mice. A, Immunohistochemical staining for CD31 showed more vessels in the tumors from the Ang-2-transfected group than in the tumors from the other three groups (mean values ±SE). Tumors produced by cells transfected with Ang-1 had significantly fewer blood vessels than the other groups. *P<0.05 versus HT29, pcDNA, and Ang-2 groups (student's t-test). ♦P<0.05 versus HT29, pcDNA, and Ang-1 groups (student's t-test). B, Immunohistochemical staining for PCNA showed higher tumor cell proliferation in the tumors from the Ang-2-transfected group than in the tumors from the other three groups (mean values ±SE). Tumors produced by cells transfected with Ang-1 had less tumor cell proliferation than tumors from the other groups. *P<0.05 versus HT29, pcDNA, and Ang-2 groups. ♦P<0.05 versus HT29, pcDNA, and Ang-1 groups.

FIG. 3 shows the effect of overexpression of Ang-1 or Ang-2 on SQ tumor growth.

FIG. 4 demonstrates final mouse weight after 37 days of liver tumor growth in the presence of Ang-1 or vector control.

FIG. 5 shows liver weight on day 37 following introduction of Ang-1 or vector control.

FIG. 6 demonstrates the effect of Ang-1 overexpression on liver tumor volume (mm³).

FIG. 7 shows vessel counts in HT29 colon cancer liver tumors following Ang-1 overexpression compared to vector control.

FIG. 8 shows an intradermal Miles assay illustrating the effect of Ang-1, Ang-2, PBS, VEGF, and pcDNA on vascular permeability.

FIG. 9 demonstrates the effect of Ang-1 on vascular permeability determined by area of leakage.

FIGS. 10A and 10B demonstrate the effect of Ang-1 expression on hepatic tumor growth. HT29 colon cancer cells transfected with a vector containing Ang-1 or an empty pcDNA vector were injected directly into the liver of nude mice to form hepatic tumors. After 37 days of tumor growth, livers were excised and liver weight and tumor diameters were determined. FIG. 10A shows overexpression of Ang-1 significantly reduced tumor burden (reflected by liver weight) relative to control (*P<0.05, Student's t-test). FIG. 10B shows that volumes of Ang-1 tumors were significantly smaller (*P<0.05, Mann-Whitney U test) than those of tumors in the pcDNA group (tumor volume=width²×length×0.5). Bars, SEM.

FIGS. 11A and 11B show the effect of Ang-1 overexpression on vessel density and tumor cell proliferation. Tumor sections were stained for CD31 for vessel counts and for PCNA for percentage of proliferating tumor cells. FIG. 11A demonstrates that fewer vessels were present in Ang-1-transfected tumors than in pcDNA-transfected tumors (*P<0.03). FIG. 11B shows that fewer proliferating cells were present in the Ang-1-transfected group than in the control (pcDNA) group (*P<0.01, Student's t-test). Bars, SEM.

FIG. 12 demonstrates the effect of Ang-1 overexpression on pericyte coverage of tumor vessels. Ang-1-transfected tumors developed a significantly higher percentage of pericyte-covered vessels than did pcDNA tumors (*P<0.01, Student's t-test). Bars, SEM.

FIG. 13 shows the effect of Ang-1 secretion by tumor cells on hepatic tumor growth of pcDNA cells. Various mixtures of Ang-1 and pcDNA transfected cells were injected into the liver as described herein. After 35 days of tumor growth, livers were excised and tumor growth was determined. Average tumor volumes in livers of mice from the 50% Ang-1, 10% Ang-1 and 100% Ang-1 group were significantly lower (*P<0.04 for all, Mann-Whitney U test) than those of tumors in the 100% pcDNA group. Bars, SEM.

FIG. 14 demonstrates the effect of Ang-1 on vessel density in an in vivo angiogenesis assay. Agarose-Gelfoam sponges containing either Ang-1 (Ang-1 TFD; 1.0 μg/ml) or PBS (control) were implanted subcutaneously into mice, where they remained for 14 days before being harvested, sectioned, and stained. FIG. 14 shows that Ang-1 TFD significantly reduced vessel density in subcutaneously implanted Gelfoam sponges as compared with control (PBS-treated) sponges (*P<0.01, Student's t-test). Bars, SEM.

FIG. 15 shows the effect of conditioned media of Ang-1-transfected or pcDNA-transfected cells on vascular permeability. Conditioned medium from transfected cells was collected in serum-reduced MEM after 48 h of culture, centrifuged, and filter-sterilized. The effects of this conditioned medium on vascular permeability were investigated with an intradermal Miles assay using Evans blue dye (0.5%). VEGF (10 ng/ml) served as positive control. Densitometry of harvested dorsal skin (20 min after intradermal injection) showed significantly lower dye density at CM-Ang-1 injection sites than at CM-pcDNA or VEGF injection sites (*P<0.01, Student's t-test). Representative images of the skin from one mouse are shown. Bars, SEM.

### DETAILED DESCRIPTION OF THE INVENTION

In keeping with long-standing patent law convention, the words “a” and “an” when used in the present specification in concert with the word comprising, including the claims, denote “one or more.”

The practice of the present invention will employ, unless otherwise indicated, conventional techniques of molecular biology, microbiology, recombinant DNA, and so forth which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

I. Definitions

The term “angiogenesis” refers to the generation of new blood vessels into cells, tissue, organs or tumors. In a specific embodiment, the new blood vessels generate from existing vessels.

The term “contacting” is used herein interchangeably with the following: combined with, added to, mixed with, passed over, incubated with, flowed over, put into, and so forth. Moreover, the compounds of the present invention can be “administered” by any conventional method such as, for example, parenteral, oral, topical and inhalation routes well known in the art.

“An amount sufficient,” “an effective amount,” “therapeutically effective amount” or “anti-angeogenic” amount refer to an amount of a compound or composition effective to depress, suppress or inhibit angiogenesis or result in amelioration of symptoms associated with an angiogenic disease. The desired result can be either a subjective relief of a symptom(s) or an objectively identifiable improvement in the recipient of the dosage, a decrease in the vascularization of of the intended tissue, such as a tumor, or a decrease in the rate of angiogenesis as noted by a clinician or other qualified observer.

The terms “treating cancer,” “therapy,” and the like refer generally to any improvement in the mammal having the cancer wherein the improvement can be ascribed to treatment with the compounds of the present invention. The improvement can be either subjective or objective. For example, if the mammal is human, the patient may note improved vigor or vitality or decreased pain as subjective symptoms of improvement or response to therapy. Alternatively, the clinician may notice decrease in tumor size or tumor burden based on physical exam, laboratory parameters, tumor markers or radiographic findings. Some laboratory signs that the clinician may observe for response to therapy include normalization of tests such as white blood cell count, red blood cell count, platelet count, erythrocyte sedimentation rate, and various enzyme levels. Additionally, the clinician may observe a decrease in a detectable tumor marker. Alternatively, other tests can be used to evaluate objective improvement such as sonograms, nuclear magnetic resonance testing and positron emissions testing.

“Inhibiting the growth of tumor cells” can be evaluated by any accepted method of measuring whether growth of the tumor cells has been slowed or diminished. This includes direct observation and indirect evaluation such as subjective symptoms or objective signs as discussed above.

II. The Present Invention

The process of angiogenesis is essential for tumor growth and metastases formation and has been associated with aggressive disease in, for example, human colorectal cancer (Folkman, 1992; Folkman, 1995; Fidler et al., 2000; Takahashi et al., 1995; Takahashi et al., 1998). Pro-angiogenic factors, such as VEGF,³ promote endothelial cell (EC) proliferation, invasion, and angiogenesis. However, the activity of angiogenic factors is modulated by other factors that affect EC survival and attachment to surrounding structures. Recently, the angiopoietins (Ang-1 to Ang-4) have been shown to be important mediators of angiogenesis by their regulation of EC cell survival in malignant and non-malignant tissues (Peters, 1998). Ang-1 has been identified as a major activator of the tyrosine kinase receptor Tie-2 (Tek), resulting in a downstream activation of the PI-3K/Akt survival pathway, thereby promoting endothelial cell survival (Papapetropoulos et al., 2000). Ang-2 is the naturally occurring antagonist to Ang-1 and prevents Tie-2 activation; this effect leads to vessel destabilization, a necessary step in the initiation of angiogenesis by VEGF (Maisonpierre et al., 1997). Balanced and sequential expression of angiopoietins and VEGF is required for successful angiogenesis (Asahara et al., 1998; Ray et al., 2000). Ang-1 has also been shown to override VEGF-mediated effects on vascular permeability (vessel leakage) (Thurston et al., 1999; Thurston et al., 2000).

In human colorectal cancer, angiopoietins appear to be expressed differently in tumors and non-malignant tissues (Ahmad et al., 2001). Ang-2 is expressed ubiquitously in tumor epithelium of human colon cancer specimens, whereas expression of Ang-1 in tumor epithelium is rarely detected. This observation suggests that a net gain in Ang-2 activity over Ang-1 activity might be an initiating factor for tumor angiogenesis (Audero et al., 2001; Mitsutake et al., 2002; Pomyje et al., 2001).

Although several investigators have shown that loss of Ang-1 activity may augment tumor angiogenesis, others have suggested that Ang-1 is pro-angiogenic (Ahmad et al., 2001; Liu et al., 2001; Bruns et al., 2000; Shaheen et al., 2001; Davis et al., 2003; McCarty et al., 2002; Eberhard et al., 2000). Thus, the effects of angiopoietins on angiogenesis and tumor growth remain controversial. The present inventors have previously demonstrated that imbalances in angiopoietin expression significantly affected angiogenesis and tumor growth of subcutaneously implanted colon cancer cells (HT29) (Ahmad et al., 2001). In that study, Ang-1 overexpression inhibited angiogenesis and tumor growth of subcutaneous xenografts.

In the present invention, the present inventors identified that overexpression of Ang-1 by tumor cells impairs angiogenesis and thereby inhibits tumor growth of human colon cancer cells, exemplified by those implanted into livers of nude mice (the liver being the most common site of colon cancer metastases). The angiogenic effect of a novel recombinant Ang-1 (Ang-1 TFD) in an in vivo angiogenesis assay is described herein, as is characterization of the role of Ang-1 in vascular permeability and pericyte coverage.

As explained above, the present invention relates to the discovery that the compounds and methods of the present invention are useful for inhibiting angiogenesis and, in turn, for treating diseases associated with undesired angiogenesis. As such, in one embodiment, the present invention provides a method of inhibiting undesired angiogenesis in a cell, the method comprising contacting the cell with an effective amount, i.e., an anti-angiogenic amount, of an angiopoietin-1 compound. In another embodiment, the present invention provides a method of inhibiting the vascularization of endothelial cells, the method comprising contacting a cell, tissue or organ containing the endothelial cells with an effective amount of an angiopoietin compound. In a presently preferred embodiment, the cells are in a mammalian subject.

This invention relates to a method of treating mammalian diseases associated with undesired and uncontrolled angiogenesis, the method comprising administering to a mammal an anti-angiogenic compound of angiopoietin-1 in an amount, i.e., a dosage, sufficient to inhibit angiogenesis. The particular dosage of a compound of angiopoietin-1 required to inhibit angiogenesis and/or angiogenic diseases will depend upon the severity of the condition, the route of administration, and related factors that will be decided by the attending health care provider. Generally, accepted and effective daily doses will be the amount sufficient to effectively inhibit angiogenesis and/or angiogenic diseases.

The methods of treatment provided by this invention are practiced by administering to a mammal in need thereof a dose of a compound of angiopoietin-1 (or a pharmaceutically acceptable salt or solvate thereof) that is effective to inhibit angiogenesis and/or angiogenic diseases. The term “inhibit” is used herein to include its generally accepted meaning which includes prophylactically treating a human subject to incurring angiogenesis and/or angiogenic diseases, and holding in check and/or treating existing angiogenesis and/or angiogenic diseases. As such, the present invention includes both medical therapeutic and/or prophylactic treatment, as appropriate.

The methods of the present invention can be used to treat a wide variety of diseases, including cancer. Diseases associated with, for example, corneal neovascularization that can be treated using the methods of the present invention include, but are not limited to, diabetic retinopathy, retinopathy of prematurity, corneal graft rejection, neovascular glaucoma and retrolental fibroplasia, epidemic keratoconjunctivitis, vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical bums, bacterial ulcers, fungal ulcers, Herpes simplex infections, Herpes zoster infections, protozoan infections, Kaposi sarcoma, Mooren ulcer, Terrien's marginal degeneration, marginal keratolysis, trauma, rheumatoid arthritis, systemic lupus, polyarteritis, Wegeners sarcoidosis, Scleritis, Steven's Johnson disease, periphigoid radial keratotomy, and corneal graph rejection.

Diseases associated with retinal/choroidal neovascularization that can be treated using the methods of the present invention include, but are not limited to, diabetic retinopathy, macular degeneration, sickle cell anemia, sarcoid, syphilis, pseudoxanthoma elasticurn, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications. Other diseases include, but are not limited to, diseases associated with rubeosis (neovasculariation of the angle) and diseases caused by the abnormal proliferation of fibrovascular or fibrous tissue, including all forms of proliferative vitreoretinopathy, whether or not associated with diabetes.

Diseases associated with chronic inflammation can also be treated using the methods of the present invention. Diseases with symptoms of chronic inflammation include, but are not limited to, inflammatory bowel diseases, such as Crohn's disease and ulcerative colitis, psoriasis, sarcoidosis and rheumatoid arthritis. Unwanted or uncontrolled angiogenesis is a key element that these chronic inflammatory diseases all have in common. The chronic inflammation depends on continuous formation of capillary sprouts to maintain an influx of inflammatory cells. The influx and presence of the inflammatory cells produce granulomas and, thus, maintain the chronic inflammatory state. Inhibition of angiogenesis using the compositions and methods of the present invention prevents the formation of the granulomas, thereby alleviating the disease.

As mentioned above, the methods of the present invention can be used to treat patients with inflammatory bowel diseases, such as Crohn's disease and ulcerative colitis. Crohn's disease occurs as a chronic transmural inflammatory disease that most commonly affects the distal ileum and colon, but may also occur in any part of the gastrointestinal tract from the mouth to the anus and perianal area. Patients with Crohn's disease generally have chronic diarrhea associated with abdominal pain, fever, anorexia, weight loss and abdominal swelling. Prevention of angiogenesis by the compositions and methods of the present invention inhibits the formation of the sprouts and prevents the formation of granulomas. Ulcerative colitis is also a chronic, nonspecific, inflammatory and ulcerative disease arising in the colonic mucosa and is characterized by the presence of bloody diarrhea.

The inflammatory bowel diseases also exhibit extra intestinal manifestations, such as skin lesions. Such lesions are characterized by inflammation and angiogenesis and can occur at many sites other than in the gastrointestinal tract. The compositions and methods of the present invention can also be used to treat these lesions by preventing the angiogenesis, thus reducing the influx of inflammatory cells and the lesion formation.

Sarcoidosis is another chronic inflammatory disease that is characterized as a multisystem granulomatous disorder. The granulomas of this disease can form anywhere in the body and, thus, the symptoms depend on the site of the granulomas and whether the disease active. The granulomas are created by the angiogenic capillary sprouts providing a constant supply of inflammatory cells. The compounds and method of this invention can be used to treat sarcoidosis.

The methods of the present invention can also be used to treat the chronic inflammatory conditions associated with psoriasis. Psoriasis, a skin disease, is another chronic and recurrent disease that is characterized by papules and plaques of various sizes. Prevention of the formation of the new blood vessels necessary to maintain the characteristic lesions leads to relief from the symptoms.

Another disease which can be treated using the methods of the present invention is rheumatoid arthritis. Rheumatoid arthritis is a chronic inflammatory disease characterized by nonspecific inflammation of the peripheral joints. It is thought that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Other diseases that can be treated using the methods of the present invention are hemangiomas, Osler-Weber-Rendu disease, or hereditary hemorrhagic telangiectasia, solid or blood borne tumors and acquired immune deficiency syndrome.

The methods of this invention are effective in inhibiting angiogenesis associated with malignant tumor growth. This includes cancerous tumor growth on cells tissues and organs. The methods of the present invention are useful in treating the growth of a number of tumor cells and for treating a wide variety of cancers. Such tumor cells include, by way of example and not limitation, lung, colon, breast, ovarian, prostate and hepatic tumor cells as well as squamous cell carcinomas. Such cancers include, by way of example and not limitation, carcinomas such as pharynx, colon, rectal, pancreatic, stomach, liver, lung, breast, skin, prostate, ovary, cervical, uterine and bladder cancers; leukemias; lymphomas; gliomas; retinoblastomas; and sarcomas.

In an additional related embodiment, a tissue to be treated is a tumor tissue of a patient with a solid tumor, a metastases, a skin cancer, a hemangioma or angiofibroma and the like cancer, and the angiogenesis to be inhibited is tumor tissue angiogenesis where there is neovascularization of a tumor tissue.

Inhibition of tumor tissue angiogenesis is a particularly preferred embodiment because of the important role neovascularization plays in tumor growth. In the absence of neovascularization of tumor tissue, the tumor tissue does not obtain the required nutrients, slows in growth, ceases additional growth, regresses and ultimately becomes necrotic resulting in killing of the tumor. Stated in other words, the present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because (1) their formation requires vascularization of a primary tumor so that the metastatic cancer cells can exit the primary tumor and (2) their establishment in a secondary site requires neovascularization to support growth of the metastases.

In a related embodiment, the invention contemplates the practice of the method in conjunction with other therapies such as conventional chemotherapy directed against solid tumors and for control of establishment of metastases. The administration of angiogenesis inhibitor is typically conducted during or after chemotherapy, although it is preferably to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases.

The present method for inhibiting angiogenesis in a tissue comprises contacting a tissue in which angiogenesis is occurring, or is at risk for occurring, with a composition comprising a therapeutically effective amount of an angiopoietin-1 compound.

The dosage ranges for the administration of the angiopoietin-1 compound depend upon the form of the compound, and its potency, as described further herein, and are amounts large enough to produce the desired effect in which angiogenesis and the disease symptoms mediated by angiogenesis are ameliorated. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

A therapeutically effective amount is an amount of an angiopoietin-1 compound sufficient to produce a measurable inhibition of angiogenesis in the tissue being treated, i.e., and angiogenesis-inhibiting amount. Inhibition of angiogenesis can be measured in situ by immunohistochemistry, as described herein, or by other methods known to one skilled in the art.

III. Definitions and Techniques Affecting Gene Products and Genes

A. Angiopoietin-1 Gene Products and Genes

In this patent the terms “angiopoietin-1 gene product” and “angiopoietin-1” refer, in some embodiments, to proteins and polypeptides having amino acid sequences which are substantially identical to the native angiopoietin-1 amino acid sequences or which are biologically active in that they are capable of binding to receptors or cross-reacting with anti-angiopoietin-1 antibody raised against angiopoietin-1.

Some examples of angiopoietin-1 polypeptides useful in the present invention include (noted by their National Center for Biotechnology Information's GenBank database Accession Number): AAB50557 (SEQ ID NO:13); AAB50558 (SEQ ID NO:14); AAC61872 (SEQ ID NO:15); AAC78246 (SEQ ID NO:16); AAC78245 (SEQ ID NO:17); NP_{—}001137 (SEQ ID NO:18); AAG34113 (SEQ ID NO:19); AAK14992 (SEQ ID NO:20); AAK31330 (SEQ ID NO:21); AAL13077 (SEQ ID NO:22); AAK83347 (SEQ ID NO:23); NP_{—}033770 (SEQ ID NO:24); 035460 (SEQ ID NO:25); 008538 (SEQ ID NO:26); 018920 (SEQ ID NO:27); Q15389 (SEQ ID NO:28); XP_{—}204878 (SEQ ID NO:35); NP_{—}647451 (SEQ ID NO:36); NP_{—}445998 (SEQ ID NO:37); BAC10290 SEQ ID NO:38); AAM92271 (SEQ ID NO:39); AAM81745 (SEQ ID NO:40); AAH29406 (SEQ ID NO:41); BAB91325 (SEQ ID NO:42); and NP_{—}571888 (SEQ ID NO:43).

A skilled artisan recognizes that angiopoietin-1 comprises a modular structure comprising a receptor-binding domain, a dimerization motif, and a superclustering motif able to form varable-sized multimers (Davis et al., 2002). In specific embodiments, a C-terminal domain (similar to fibrinogen) of Ang1 is required for receptor binding, a central coiled-coil domain is capable of dimerizing the C-terminal domains, and a short N-terminal region forms ring-like structures that supercluster dimers into varable-sized multimers. In specific embodiments of the present invention, a therapeutic agent comprising angiopoietin-1, wherein the angiopoietin-1 comprises one or more of said domains/motifs, is utilized. A skilled artisan recognizes that the beginning of the coiled-coil and F (fibrinogen) domains are defined by the amino acid sequences FSSQKLQH (SEQ ID NO:55) and FRDCADVY (SEQ ID NO:56), respectively.

In specific embodiments, the domains/motifs utilized in the present invention provide activity to the angiopoietin-1. In some embodiments, this activity comprises Tie2 binding activity, the ability to inhibit angiogenesis, to inhibit cell growth, to stabilize the endothelium associated with a tumor, to prevent endothelial cell proliferation associated with a tumor, or a combination thereof. In some embodiments of the present invention, Ang1 acts in a multimeric state, such as by dimers, trimers, tetramers, and so on. In a particular embodiment, Ang1 in a tetrameric state (or higher) is utilized.

The term “angiopoietin-1 gene product” also includes analogs of angiopoietin-1 molecules which exhibit at least some biological activity in common with native angiopoietin-1. Such analogs include, but are not limited to, truncated angiopoietin-1 polypeptides and angiopoietin-1 polypeptides having fewer amino acids than native angiopoietin-1. Furthermore, those skilled in the art of mutagenesis will appreciate that homologs to the mouse angiopoietin-1 gene, including human homologs, which homologes are as yet undisclosed or undiscovered, may be used in the methods and compositions disclosed herein.

The invention contemplates a mini-angiopoietin-1 gene product comprising at least the N-terminal domain of an angiopoietin-1 gene product. Such a mini-angiopoietin-1 gene product may further comprise a spacer domain and/or a C-terminal domain of the angiopoietin-1-gene product.

The term “angiopoietin-1 gene” or “angiopoietin-1 polynucleotide” refers to any DNA sequence that is substantially identical to a DNA sequence encoding an angiopoietin-1 gene product as defined above. The term also refers to RNA, or antisense sequences compatible with such DNA sequences. A “angiopoietin-1 gene” may also comprise any combination of associated control sequences.

Some examples of angiopoietin-1 polynucleotides useful in the present invention include (noted by their National Center for Biotechnology Information's GenBank database Accession Number): U83508 (SEQ ID NO:1); U83509 (SEQ ID NO:2); AF093573 (SEQ ID NO:3); AF030376 (SEQ ID NO:4); AF032923 (SEQ ID NO:5); NM_{—}001146 (SEQ ID NO:6); AF311727 (SEQ ID NO:7); AF233227 (SEQ ID NO:8); AF345932 (SEQ ID NO:9); AY052399 (SEQ ID NO:10); AF379602 (SEQ ID NO:11); NM_{—}009640 (SEQ ID NO:12); NM_{—}004673 (SEQ ID NO:44); NM_{—}053546 (SEQ ID NO:45); NM_{—}139290 (SEQ ID NO:46); XM_{—}204878 (SEQ ID NO:47); AB080023 (SEQ ID NO:48); AY124380 (SEQ ID NO:49); AY121504 (SEQ ID NO:50); BC029406 (SEQ ID NO:51); AB084454 (SEQ ID NO:52); AB084284 (SEQ ID NO:53); and NM_{—}131813 (SEQ ID NO:54).

The term “substantially identical”, when used to define either a angiopoietin-1 amino acid sequence or angiopoietin-1 gene polynucleotide sequence, means that a particular subject sequence, for example, a mutant sequence, varies from the sequence of natural angiopoietin-1 by one or more substitutions, deletions, or additions, the net effect of which is to retain at least some biological activity of the angiopoietin-1 protein. Alternatively, DNA analog sequences are “substantially identical” to specific DNA sequences disclosed herein if: (a) the DNA analog sequence is derived from coding regions of the natural angiopoietin-1 gene; or (b) the DNA analog sequence is capable of hybridization of DNA sequences of (a) under moderately stringent conditions and which encode biologically active angiopoietin-1; or (c) DNA sequences which are degenerative as a result of the genetic code to the DNA analog sequences defined in (a) or (b). Substantially identical analog proteins will be greater than about 80% similar to the corresponding sequence of the native protein. Sequences having lesser degrees of similarity but comparable biological activity are considered to be equivalents. In determining polynucleotide sequences, all subject polynucleotide sequences capable of encoding substantially similar amino acid sequences are considered to be substantially similar to a reference polynucleotide sequence, regardless of differences in codon sequence.

B. Percent Similarity

Percent similarity may be determined, for example, by comparing sequence information using the GAP computer program, available from the University of Wisconsin Geneticist Computer Group. The GAP program utilizes the alignment method of Needleman et al., 1970, as revised by Smith et al., 1981. Briefly, the GAP program defines similarity as the number of aligned symbols (i.e. nucleotides or amino acids) which are similar, divided by the total number of symbols in the shorter of the two sequences. The preferred default parameters for the GAP program include (1) a unitary comparison matrix (containing a value of 1 for identities and 0 for non-identities) of nucleotides and the weighted comparison matrix of Gribskov et al., 1986, (2) a penalty of 3.0 for each gap and an additional 0.01 penalty for each symbol and each gap; and (3) no penalty for end gaps.

C. Polynucleotide Sequences

In certain embodiments, the invention concerns the use of angiopoietin-1 genes and gene products, such as the angiopoietin-1 that includes a sequence which is essentially that of the known angiopoietin-1 gene, or the corresponding protein. The term “a sequence essentially as angiopoietin-1” means that the sequence substantially corresponds to a portion of the angiopoietin-1 gene and has relatively few bases or amino acids (whether DNA or protein) which are not identical to those of angiopoietin-1 (or a biologically functional equivalent thereof, when referring to proteins). The term “biologically functional equivalent” is well understood in the art and is further defined in detail herein. Accordingly, sequences which have between about 70% and about 80%; or more preferably, between about 81% and about 90%; or even more preferably, between about 91% and about 99%; of amino acids which are identical or functionally equivalent to the amino acids of angiopoietin-1 will be sequences which are “essentially the same”.

Angiopoietin-1 genes which have functionally equivalent codons are also covered by the invention. The term “functionally equivalent codon” is used herein to refer to codons that encode the same amino acid, such as the six codons for arginine or serine, and also refers to codons that encode biologically equivalent amino acids (Table 1).

TABLE 1
FUNCTIONALLY EQUIVALENT CODONS.

| Amino Acids | Codons | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic Acid | Asp | D | GAC | GAU | | | | |
| Glutamic Acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCU | | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

It will also be understood that amino acid and polynucleotide sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to polynucleotide sequences which may, for example, include various non-coding sequences flanking either of the 5′ or 3′ portions of the coding region or may include various internal sequences, i.e., introns, which are known to occur within genes.

In certain embodiments, the invention concerns the use of truncated angiopoietin-1 genes or polynucleotide sequences that encode an angiopoietin-1 polypeptide with less amino acids than native angiopoietin-1. The present invention also encompasses the use of DNA segments which are complementary, or essentially complementary, to the sequences set forth in the specification. Polynucleotide sequences which are “complementary” are those which are capable of base-pairing according to the standard Watson-Crick complementarily rules. As used herein, the term “complementary sequences” means polynucleotide sequences which are substantially complementary, as may be assessed by the same nucleotide comparison set forth above, or as defined as being capable of hybridizing to the polynucleotide segment in question under relatively stringent conditions such as those described herein.

D. Biologically Functional Equivalents

As mentioned above, modification and changes may be made in the structure of angiopoietin-1 and still obtain a molecule having like or otherwise desirable characteristics. For example, certain amino acids may be substituted for other amino acids in a protein structure without appreciable loss of interactive binding capacity with angiopoietin-1 ligands or receptors. Since it is the interactive capacity and nature of a protein that defines that protein's biological functional activity, certain amino acid sequence substitutions and/or deletions can be made in a protein sequence (or, of course, its underlying DNA coding sequence) and nevertheless obtain a protein with like or even countervailing properties (e.g., antagonistic v. agonistic). It is thus contemplated by the inventors that various changes may be made in the sequence of the angiopoietin-1 proteins or peptides (or underlying DNA) without appreciable loss of their biological utility or activity. Included in such changes are truncated angiopoietin-1 polypeptides and angiopoietin-1 polypeptides having less amino acid residues than native angiopoietin-1.

It is also well understood by the skilled artisan that, inherent in the definition of a biologically functional equivalent protein or peptide, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted. Of course, a plurality of distinct proteins/peptides with different substitutions may easily be made and used in accordance with the invention.

It is also well understood that where certain residues are shown to be particularly important to the biological or structural properties of a protein or peptide, e.g., residues in active sites, such residues may not generally be exchanged. This is the case in the present invention, where any changes in angiopoietin-1 that render the polypeptide incapable of inhibiting tumor cell proliferation or incapable of inhibiting angiogenesis would result in a loss of utility of the resulting peptide for the present invention.

Amino acid substitutions, such as those which might be employed in modifying angiopoietin-1 are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alaanine, glycine and serine; and phenylalanine, tryptophan and tyrosine; are defined herein as biologically functional equivalents.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteinelcystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within .+−0.1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0.+−0.1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid.

E. Sequence Modification Techniques

Modifications to the angiopoietin-1 peptides may be carried out using techniques such as site directed mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art as exemplified by publications (Adelman et al., 1983). As will be appreciated, the technique typically employs a phage vector which exists in both a single stranded and double stranded form. Typical vectors useful in site-directed mutagenesis include vectors such as the M13 phage (Messing et al., 1981). These phage are readily commercially available and their use is generally well known to those skilled in the art. Double stranded plasmids are also routinely employed in site directed mutagenesis which eliminates the step of transferring the gene of interest from a plasmid to a phage.

In general, site-directed mutagenesis in accordance herewith is performed by first obtaining a single-stranded vector or melting apart the two strands of a double stranded vector which includes within its sequence a DNA sequence which encodes the angiopoietin-1 gene. An oligonucleotide primer bearing the desired mutated sequence is prepared, generally synthetically, for example by the method of Crea et al. (1978). This primer is then annealed with the single-stranded vector, and subjected to DNA polymerizing enzymes such as g polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected gene using site-directed mutagenesis is provided as a means of producing potentially useful angiopoietin-1 and is not meant to be limiting as there are other ways in which sequence variants of these peptides may be obtained. For example, recombinant vectors encoding the desired genes may be treated with mutagenic agents to obtain sequence variants (see, e.g., a method described by Eichenlaub, 1979) for the mutagenesis of plasmid DNA using hydroxylamine.

F. Antisense Constructs

In some cases, mutant tumor suppressors may not be non-functional. Rather, they may have aberrant functions that cannot be overcome by replacement gene therapy, even where the “wild-type” molecule is expressed in amounts in excess of the mutant polypeptide. Antisense treatments are one way of addressing this situation. Antisense technology also may be used to “knock-out” function of angiopoietin-1 in the development of cell lines or transgenic mice for research, diagnostic and screening purposes.

Antisense methodology takes advantage of the fact that nucleic acids tend to pair with “complementary” sequences. By complementary, it is meant that polynucleotides are those which are capable of base-pairing according to the standard Watson-Crick complementarily rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarily to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

G. Synthetic Polypeptides

The present invention also describes angiopoietin-1 proteins and related peptides for use in various embodiments of the present invention. The angiopoietin-1 polypeptide may have fewer amino acids than native angiopoietin-1. Relatively small peptides can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

H. Other Structural Equivalents

In addition to the angiopoietin-1 peptidyl compounds described herein, the inventors also contemplate that other sterically similar compounds may be formulated to mimic the key portions of the peptide structure. Such compounds may be used in the same manner as the peptides of the invention and hence are also functional equivalents. The generation of a structural functional equivalent may be achieved by the techniques of modeling and chemical design known to those of skill in the art. It will be understood that all such sterically similar constructs fall within the scope of the present invention.

IV. Expression Vectors

In certain aspects of the present invention it may be necessary to express the angiopoietin-1 proteins. Throughout this application, the term “expression construct” is meant to include any type of genetic construct containing a polynucleotide coding for a gene product in which part or all of the polynucleotide encoding sequence is capable of being transcribed. The transcript may be translated into a protein, but it need not be. Thus, in certain embodiments, expression includes both transcription of an angiopoietin-1 gene and translation of an angiopoietin-1 mRNA into an angiopoietin-1 protein product. In other embodiments, expression only includes transcription of the polynucleotide encoding an angiopoietin-1 or its complement.

In order for the construct to effect expression of at least an angiopoietin-1 transcript, the polynucleotide encoding the angiopoietin-1 polynucleotide will be under the transcriptional control of a promoter. A “promoter” refers to a DNA sequence recognized by the synthetic machinery of the host cell, or introduced synthetic machinery, that is required to initiate the specific transcription of a gene. The phrase “under transcriptional control” means that the promoter is in the correct location in relation to the polynucleotide to control RNA polymerase initiation and expression of the polynucleotide.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of an angiopoietin-1 polynucleotide is not believed to be critical, so long as it is capable of expressing the polynucleotide in the targeted cell at sufficient levels. Thus, where a human cell is targeted, it is preferable to position the polynucleotide coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter and the Rous sarcoma virus long terminal repeat can be used to obtain high-level expression of the angiopoietin-1 polynucleotide. The use of other viral or mammalian cellular or bacterial phage promoters which are well-known in the art to achieve expression of polynucleotides is contemplated as well, provided that the levels of expression are sufficient to produce a growth inhibitory effect.

By employing a promoter with well-known properties, the level and pattern of expression of a polynucleotide following transfection can be optimized. For example, selection of a promoter which is active in specific cells, such as tyrosinase (melanoma), alpha-fetoprotein and albumin (liver tumors), CC10 (lung tumor) and prostate-specific antigen (prostate tumor) will permit tissue-specific expression of angiopoietin-1 polynucleotides. Table 2 lists several elements/promoters which may be employed, in the context of the present invention, to regulate the expression of angiopoietin-1 constructs. This list is not intended to be exhaustive of all the possible elements involved in the promotion of angiopoietin-1 expression but, merely, to be exemplary thereof

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a angiopoietin-1 construct. Use of a T3, T7 or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional genetic expression vector.

TABLE 2

ENHANCER
Immunoglobulin Heavy Chain
Immunoglobulin Light Chain
T-Cell Receptor
HLA DQ α and DQ β
β-Interferon
lnterleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
c-fos
c-HA-ras
Insulin
Neural Cell Adhesion Molecule (NCAM)
α₁-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

Further, selection of a promoter that is regulated in response to specific physiologic signals can permit inducible expression of the angiopoietin-1 construct. For example, with the polynucleotide under the control of the human PAI-1 promoter, expression is inducible by tumor necrosis factor. Table 3 illustrates several promoter/inducer combinations:

TABLE 3

| Element | Inducer |
|---|---|
| MT 11 | Phorbol Ester (TFA) Heavy metals |
| MMTV (mouse mammary | Glucocorticoids |
| | tumor virus) |
| β-Interferon | Poly(rI)X |
| | Poly(rc) |
| Adenovirus 5 E2 | Ela |
| c-jun | Phorbol Ester (TPA), H₂ O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | JL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone | Thyroid Hormone |
| | α Gene |

In certain embodiments of the invention, the delivery of an expression vector in a cell may be identified in vitro or in vivo by including a marker in the expression vector. The marker would result in an identifiable change to the transfected cell permitting easy identification of expression. Usually the inclusion of a drug selection marker aids in cloning and in the selection of transformants. Alternatively, enzymes such as herpes simplex virus thymidine kinase (tk) (eukaryotic) or chloramphenicol acetyltransferase (CAT) (prokaryotic) may be employed. Immunologic markers also can be employed. The selectable marker employed is not believed to be important, so long as it is capable of being expressed along with the polynucleotide encoding angiopoietin-1. Further examples of selectable markers are well known to one of skill in the art.

One typically will include a polyadenylation signal to effect proper polyadenylation of the transcript. The nature of the polyadenylation signal is not believed to be crucial to the successful practice of the invention, and any such sequence may be employed. The inventor has employed the SV40 polyadenylation signal in that it was convenient and known to function well in the target cells employed. Also contemplated as an element of the expression construct is a terminator. These elements can serve to enhance message levels and to minimize read through from the construct into other sequences.

The expression construct may comprise a virus or engineered construct derived from a viral genome. The ability of certain viruses to enter cells via receptor-mediated endocytosis and, in some cases, integrate into the host cell chromosomes, have made them attractive candidates for gene transfer in to mammalian cells. However, because it has been demonstrated that direct uptake of naked DNA, as well as receptor-mediated uptake of DNA complexes, expression vectors need not be viral but, instead, may be any plasmid, cosmid or phage construct that is capable of supporting expression of encoded genes in mammalian cells, such as pUC or Bluescript™ plasmid series.

V. Rational Drug Design

The goal of rational drug design is to produce structural analogs of biologically active polypeptides or compounds with which they interact (agonists, antagonists, inhibitors, binding partners, etc.). By creating such analogs, it is possible to fashion drugs which are more active or stable than the natural molecules, which have different susceptibility to alteration or which may affect the function of various other molecules. In one approach, one would generate a three-dimensional structure for angiopoietin-1 or a fragment thereof. This could be accomplished by x-ray crystallograph, computer modeling or by a combination of both approaches. An alternative approach, “alanine scan,” involves the random replacement of residues throughout molecule with alanine, and the resulting affect on function determined.

It also is possible to isolate an angiopoietin-1 specific antibody, selected by a functional assay, and then solve its crystal structure. In principle, this approach yields a pharmacore upon which subsequent drug design can be based. It is possible to bypass protein crystallograph altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have improved angiopoietin-1 activity or which act as stimulators, inhibitors, agonists, antagonists or angiopoietin-1 or molecules affected by angiopoietin-1 function. By use of cloned angiopoietin-1 sequences, sufficient amounts of angiopoietin-1 can be produced to perform crystallographic studies. In addition, knowledge of the polypeptide sequences permits computer employed predictions of structure-function relationships.

The present invention also contemplates the use of angiopoietin-1 and active fragments, and nucleic acids coding therefor, in the screening of compounds for activity in either stimulating angiopoietin-1 activity, overcoming the lack of angiopoietin-1 or blocking the effect of a mutant angiopoietin-1 molecule.

The present invention also encompasses the use of various animal models. By developing or isolating mutant cells lines that fail to express normal angiopoietin-1, one can generate cancer models in mice that will be highly predictive of cancers in humans and other mammals. These models may employ the orthotopic or systemic administration of tumor cells to mimic primary and/or metastatic cancers. Alternatively, one may induce cancers in animals by providing agents known to be responsible for certain events associated with malignant transformation and/or tumor progression. Finally, transgenic animals (discussed below) that lack a wild-type angiopoietin-1 may be utilized as models for cancer development and treatment.

Treatment of animals with test compounds will involve the administration of the compound, in an appropriate form, to the animal. Administration will be by any route the could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, survival, reduction of tumor burden or mass, arrest or slowing of tumor progression, elimination of tumors, inhibition or prevention of metastasis, increased activity level, improvement in immune effector function and improved food intake.

VI. In Vivo Delivery and Treatment Protocols

Where the gene itself is employed to introduce the gene products, a convenient method of introduction will be through the use of a recombinant vector which incorporates the desired gene, together with its associated control sequences. The preparation of recombinant vectors is well known to those of skill in the art and described in many references, such as, for example, Sambrook et al. (1989), specifically incorporated herein by reference.

In vectors, it is understood that the DNA coding sequences to be expressed, in this case those encoding the angiopoietin-1 gene products, are positioned adjacent to and under the control of a promoter. It is understood in the art that to bring a coding sequence under the control of such a promoter, one generally positions the ₅′ end of the transcription initiation site of the transcriptional reading frame of the gene product to be expressed between about 1 and about 50 nucleotides “downstream” of (i.e., 3′ of) the chosen promoter. One may also desire to incorporate into the transcriptional unit of the vector an appropriate polyadenylation site (e.g., 5′-AATAAA-3′), if one was not contained within the original inserted DNA. Typically, these poly A addition sites are placed about 30 to 2000 nucleotides “downstream” of the coding sequence at a position prior to transcription termination.

While use of the control sequences of the angiopoietin-1 will be preferred, there is no reason why other control sequences could not be employed, so long as they are compatible with the genotype of the cell being treated. Thus, one may mention other useful promoters by way of example, including, e.g., an SV40 early promoter, a long terminal repeat promoter from retrovirus, an actin promoter, a heat shock promoter, a metallothionein promoter, and the like.

For introduction of the angiopoietin-1 gene, it is proposed that one will desire to preferably employ a vector construct that will deliver the desired gene to the affected cells. This will, of course, generally require that the construct be delivered to the targeted tumor cells, for example, breast, genital, or lung tumor cells. It is proposed that this may be achieved most preferably by introduction of the desired gene through the use of a viral or non viral vectors to carry the angiopoietin-1 sequences to efficiently transfect the tumor, or pretumorous tissue. This infection may be achieved preferably by liposomal delivery but may also be via adenoviral, a retroviral, a vaccinia virus, herpesvirus or adeno-associated virus vector. These vectors have been successfully used to deliver desired sequences to cells and tend to have a high infection efficiency.

Commonly used viral promoters for expression vectors are derived from polyoma, cytomegalovirus, Adenovirus 2, and Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the Hind III site toward the Bgl I site located in the viral origin of replication. Further, it is also possible, and often desirable, to utilize promoter or control sequences normally associated with the desired gene sequence, provided such control sequences are compatible with the host cell systems.

The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

A. Liposomal Transfection

Thus the expression construct may be entrapped in a liposome. Liposomes are structures created by mixing phospholipids with water, or hydration of phospholipid. The resultant bilayer structures tend to fold back upon themselves. Liposomes are frequently multilamellar, composed of concentric bilayer membranes separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

The present invention also provides particularly useful methods for introducing angiopoietin-1 gene products into cells. One method of in vivo gene transfer which can lead to expression of genes transfected into cells involves the use of liposomes. Liposomes can be used for both in vitro and in vivo transfection. Liposome-mediated gene transfer seems to have great potential for certain in vivo applications in animals (Nicolau et al., 1987). Studies have shown that intravenously injected liposomes are taken up essentially in the liver and the spleen, by the macrophages of the reticuloendothelial system. The specific cellular sites of uptake of injected liposomes appears to be mainly spleen macrophages and liver Kupffer cells. Intravenous injection of liposomes/DNA complexes can lead to the uptake of DNA by these cellular sites, and result in the expression of a gene product encoded in the DNA (Nicolau, 1982).

The inventors contemplate that angiopoietin-1 gene products can be introduced into cells using liposome-mediated gene transfer. It is proposed that such constructs can be coupled with liposomes and directly introduced via a catheter, as described by Nabel et al. (1990). By employing these methods, angiopoietin-1 gene products can be expressed efficiently at a specific site in vivo, not just the liver and spleen cells which are accessible via intravenous injection. Therefore, this invention also encompasses compositions of DNA constructs encoding a angiopoietin-1 gene product formulated as a DNA/liposome complex and methods of using such constructs.

Liposomal transfection can be via liposomes composed of, for example, phosphatidylcholine (PC), phosphatidylserine (PS), cholesterol (Chol), N-[1-(2,3-dioleyloxy)propyl]-N,N-trimethylammonium chloride (DOTMA), dioleoylphosphatidyl-ethanolamine (DOPE), and/or 3β[N-(N′N′-dimethylaminoethane)-carbarmoyl cholesterol (DC-Chol), as well as other lipids known to those of skill in the art. Those of skill in the art will recognize that there are a variety of liposomal transfection techniques which will be useful in the present invention. Among these techniques are those described in Nicolau et al., 1987, Nabel et al., 1990, and Gao et al., 1991. In one embodiment of the present invention, liposomes comprising DC-Chol and DOPE which have been prepared following the teaching of Gao et al., 1991, are used. The inventors also anticipate utility for liposomes comprised of DOTMA, such as those which are available commercially under the trademark Lipofectin.TM., from Vical, Inc., in San Diego, Calif.

Liposomes may be introduced into contact with cells to be transfected by a variety of methods. In cell culture, the liposome-DNA complex can simply be dispersed in the cell culture solution. For application in vivo, liposome-DNA complex are typically injected. Intravenous injection allow liposome-mediated transfer of DNA complex, for example, the liver and the spleen. In order to allow transfection of DNA into cells which are not accessible through intravenous injection, it is possible to directly inject the liposome-DNA complexes into a specific location in an animal's body. For example, Nabel et al. teach injection via a catheter into the arterial wall. In another example, the inventors have used intraperitoneal injection to allow for gene transfer into mice.

The present invention also contemplates compositions comprising a liposomal complex. This liposomal complex will comprise a lipid component and a DNA segment encoding an angiopoietin-1 gene.

The lipid employed to make the liposomal complex can be any of the above-discussed lipids. In particular, DOTMA, DOPE, and/or DC-Chol may form all or part of the liposomal complex. The inventors have had particular success with complexes comprising DC-Chol. In a preferred embodiment, the lipid will comprise DC-Chol and DOPE. While any ratio of DC-Chol to DOPE is expected to have utility, it is expected that those comprising a ratio of DC-Chol:DOPE between 1:20 and 20:1 will be particularly advantageous. The inventors have found that liposomes prepared from a ratio of DC-Chol:DOPE of about 1:10 to about 1:5 have been useful.

It is proposed that it will ultimately be preferable to employ the smallest region needed to suppress the angiopoietin-1 gene so that one is not introducing unnecessary DNA into cells which receive a angiopoietin-1 gene construct. Techniques well known to those of skill in the art, such as the use of restriction enzymes, will allow for the generation of small regions of angiopoietin-1. The ability of these regions to inhibit tumor cell proliferation, tumorigenicity and transformation phenotype can easily be determined by the assays reported in the Examples.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of polynucleotide in vitro and in vivo, then they are applicable for the present invention. Where a bacterial promoter is employed in the DNA construct, it also will be desirable to include within the liposome an appropriate bacterial polymerase.

B. Adenovirus

Another method for in vivo delivery involves the use of an adenovirus vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

Adenovirus is a particularly suitable gene transfer vector because of its midsized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, located at 16.8 μm is particularly efficient during the late phase of infection, and all the mnRNA's issued from this promoter possess a 5′-tripartite leader (TL) sequence which makes them preferred mRNA's for translation.

In some cases, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure. Use of the YAC system is an alternative approach for the production of recombinant adenovirus.

A particular method of introducing the angiopoietin-1 to an animal is to introduce a replication-deficient adenovirus containing the angiopoietin-1 gene. The replication-deficient construct made by E1B and E3 deletion also avoids the viral reproduction inside the cell and transfer to other cells and infection of other people, which means the viral infection activity is shut down after it infects the target cell. The angiopoietin-1 gene is still expressed inside the cells. Also, unlike retrovirus, which can only infect proliferating cells, adenovirus is able to transfer the angiopoietin-1 gene into both proliferating and non-proliferating cells. Further, the extrachromosomal location of adenovirus in the infected cells decreases the chance of cellular oncogene activation within the treated animal. A skilled artisan recognizes that a “gutless” adenoviral vector may be utilized, such as a recombinant adenoviral vector that is deleted of all Ad genes. Gutless rAVs can be propagated using a helper virus. In the most efficient system to date, an E1-deleted helper virus is used with a packaging signal that is flanked by bacteriophage P1 loxP sites (“floxed”). Infection of the helper cells that express Cre recombinase with the gutless virus together with the helper virus with a floxed packaging signal should only yield gutless rAV, as the packaging signal is deleted from the DNA of the helper virus. In another specific embodiment, a gutless vector is incapable of expressing any adenovirus antigens. An example of constructing a gutless adenoviral vector is described in U.S. Pat. No. 6,228,646. Other examples are described in Hardy et al. (1996). U.S. Pat. No. 6,156,497 is directed to the rapid generation of adenoviral vectors from which all adenovirus backbone genes have been deleted. Such gutless vectors provide a significant advance over presently available vectors because the toxicity and immunogenicity of adenoviral backbone gene products is avoided. Furthermore, a skilled artisan recognizes such a vector could incorporate up to 37,200 base pairs of heterologous sequence, as opposed to the 7,000 base pair limit incurred by standard vectors.

It is advantageous if the adenovirus vector is replication defective, or at least conditionally defective. The adenovirus may be of any of the 42 different known serotypes or subgroups A-F. Adenovirus type 5 of subgroup C is presently preferred starting material for obtaining conditional replication-defective adenovirus vector for use in the present invention. This is because Adenovirus type 5 is a human adenovirus about which the most biochemical and genetic information is known, and it has historically been used for most constructions employing adenovirus as a vector. In a specific example, in Matsubara et al. (2001) a recombinant Ad-OC-E1a was constructed using a noncollagenous bone matrix protein osteocalcin (OC) promoter to drive the viral early E1a gene with restricted replication in cells that express OC transcriptional activity. A skilled artisan is aware that this is merely exemplary, and that a particular promoter could be selected depending on the disease and target tissue which is being treated.

Introduction of the adenovirus containing the angiopoietin-1 gene product gene into a suitable host is typically done by injecting the virus contained in a buffer.

The nature of the adenovirus vector is not believed to be crucial to the successful practice of the invention. Of course, as discussed above, it is advantageous if the adenovirus vector is replication defective, or at least conditionally defective, The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotatic inoculation into the brain (Le Gal La Salle et al., 1993).

C. Retroviruses

The retroviruses are a group of single-stranded RNA viruses characterized by an ability to convert their RNA to double-stranded DNA to infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene, termed psi.components is constructed (Mann et al., 1983). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. One limitation to the use of retrovirus vectors in vivo is the limited ability to produce retroviral vector titers greater than 10⁶ infections U/mL. Titers 10- to 1,000-fold higher are necessary for many in vivo applications.

Several properties of the retrovirus have limited its use in lung cancer treatment (Stratford-Perricaudet and Perricaudet, 1991; (i) Infection by retrovirus depends on host cell division. In human cancer, very few mitotic cells can be found in tumor lesions. (ii) The integration of retrovirus into the host genome may cause adverse effects on target cells, because malignant cells are high in genetic instability. (iii) Retrovirus infection is often limited by a certain host range. (iv) Retrovirus has been associated with many malignancies in both mammals and vertebrates. (v) The titer of retrovirus, in general, is 100- to 1,000-fold lower than that of adenovirus.

D. Other Viral Vectors as Expression Constructs

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpes viruses may be employed. They offer several attractive features for various mammalian cells (Friedmann, 1989; Ridgeway, 1988; Baichwal and Sugden, 1986; Coupar et al., 1988; Howrich et al., 1990).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Cultures media containing high titers of the recombinant virus were used to infect primary duckling hepatocytes. Stable CAT gene expression was detected for at least 24 days after transfection (Chang et al., 1991).

E. Other Non-viral Vectors

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. As described above, delivery may be via viral infection where the expression construct is encapsidated in an infectious viral particle.

Several non-viral methods for the transfer of expression constructs into cultured mammalian cells also are contemplated by the present invention. These include calcium phosphate precipitation (Grahan and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the polynucleotide encoding the gene of interest may be positioned and expressed at different sites. In certain embodiments, the polynucleotide encoding the gene may be stably maintained in the cell as a separate, episomal segment of DNA. Such polynucleotide segments or “episomes” encode sequences sufficient to permit maintenance and replication independent of or in synchronization with the host cell cycle. How the expression construct is delivered to a cell and where in the cell the polynucleotide remains is dependent on the type of expression construct employed.

In one embodiment of the invention, the expression construct may simply consist of naked recombinant DNA or plasmids. Transfer of the construct may be performed by any of the methods mentioned above which physically or chemically permeabilize the cell membrane. This is particularly applicable for transfer permeabilize the cell membrane. This is particularly applicable for transfer in vitro but it may be applied to in vivo use as well. Dubensky et al. (1984) successfully injected polyomavirus DNA in the form of CaPO₄ precipitates into liver and spleen of adult and newborn mice demonstrating active viral replication and acute infection. Benvenisty and Reshef (1986) also demonstrated that direct intraperitoneal injection of CaPO₄ precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

Other expression constructs which can be employed to deliver a polynucleotide encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). A synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a polynucleotide encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a polynucleotide encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a polynucleotide into the cells, in vitro, and then the return of the modified cells back into an animal. This may involve the surgical removal of tissue/organs from an animal or the primary culture of cells and tissues. Anderson et al., U.S. Pat. No. 5,399,346, and incorporated herein in its entirety, disclose ex vivo therapeutic methods.

F. Protein Therapy

Another therapy approach is the provision, to a subject, of angiopoietin-1 polypeptide, active fragments, synthetic peptides, mimetics or other analogs thereof. The protein may be produced by recombinant expression means or, if small enough, generated by an automated peptide synthesizer. Formulations would be selected based on the route of administration and purpose including, but not limited to, liposomal formulations and classic pharmaceutical preparations.

VII. Combined Therapy Protocols

Tumor cell resistance to anti-cancer agents represents a major problem in clinical oncology. The present invention may also be used in combination with conventional therapies to improve the efficacy of chemo- and radiotherapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that angiopoietin-1 therapy could be used similarly in conjunction with chemo- or radiotherapeutic intervention.

To kill cells, such as malignant or metastatic cells, using the methods and compositions of the present invention, one would generally contact a “target” cell with a angiopoietin-1 composition and at least one anti-cancer agent. These compositions would be provided in a combined amount effective to kill or inhibit proliferation of the cell. This process may involve contacting the cells with the angiopoietin-1 composition and the anti-cancer agent(s) or factor(s) at the same time. This may be achieved by contacting the cell with a single composition or pharmacological formulation that includes both agents, or by contacting the cell with two distinct compositions or formulations, at the same time, wherein one composition includes the angiopoietin-1 composition and the other includes the anti-cancer agent.

Alternatively, the angiopoietin-1 treatment may precede or follow the anti-cancer agent treatment by intervals ranging from min to weeks. In embodiments where the anti-cancer agent and angiopoietin-1 are applied separately to the cell, one would generally ensure that a significant period of time did not expire between the time of each delivery, such that the anti-cancer agent and angiopoietin-1 composition would still be able to exert an advantageously combined effect on the cell. In such instances, it is contemplated that one would contact the cell with both agents within about 6 h to one wk of each other and, more preferably, within about 24-72 h of each other, with a delay time of only about 48 h being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the angiopoietin-1 or the anti-cancer agent will be desired. Various combinations may be employed, where angiopoietin-1 is “A” and the anti-cancer agent is “B”:

| | | | | | |
|---|---|---|---|---|---|
| A/B/A | B/A/B | B/B/A | A/A/B | B/B/B/A | B/B/A/B |
| A/A/B/B | A/B/A/B | A/B/B/A | B/B/A/A | B/A/B/A | B/A/A/B |
| A/A/A/B | B/A/A/A | A/B/A/A | A/A/B/A | A/B/B/B | B/A/B/B |

To achieve cell killing, both agents are delivered to a cell in a combined amount effective to kill the cell.

In treating cancer according to the invention, one would contact the tumor cells with a DNA damaging agent in addition to the angiopoietin-1 composition. This may be achieved by irradiating the localized tumor site with DNA damaging radiation such as X-rays, UV-light, y-rays or even microwaves. Alternatively, the tumor cells may be contacted with the DNA damaging agent by administering to the subject a therapeutically effective amount of a pharmaceutical composition comprising a DNA damaging compound such as, adriamycin, 5-fluorouracil, etoposide, camptothecin, actinomycin-D, mitomycin C, or more preferably, cisplatin. The DNA damaging agent may be prepared and used as a combined therapeutic composition, or kit, by combining it with a angiopoietin-1 composition, as described above.

Agents that directly cross-link polynucleotides, specifically DNA, are envisaged and are shown herein, to eventuate DNA damage leading to a synergistic antineoplastic combination. Agents such as cisplatin, and other DNA alkylating may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamnil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of polynucleotide precursors and subunits also lead to DNA damage. As such a number of polynucleotide precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of DNA damage, or the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventor proposes that the regional delivery of angiopoietin-1 compositions to patients with tumors will be a very efficient method for delivering a therapeutically effective gene to counteract the clinical disease. Similarly, the chemo- or radiotherapy may be directed to a particular, affected region of the subject's body. Alternatively, systemic delivery of the angiopoietin-1 or the DNA damaging agent may be appropriate in certain circumstances, for example, where extensive metastasis has occurred.

Cytokine therapy also has proven to be an effective partner for combined therapeutic regimens. Various cytokines may be employed in such combined approaches. Examples of cytokines include IL-1α IL-1β, IL-2, IL-3, IL-4, IL-5, IL-6, IL-7, IL-8, IL-9, IL-10, IL-11, IL-12, IL-13, TGF-β, GM-CSF, M-CSF, G-CSF, TNF-α, TNF-β, LAF, TCGF, BCGF, TRF, BAF, BDG, MP, LIF, OSM, TMF, PDGF, INF-α, IFN-β, IFN-γ. Cytokines are administered according to standard regimens, as described below, consistent with clinical indications such as the condition of the patient and relative toxicity of the cytokine.

A number of polypeptides are known to induce apoptosis and may be used in the combination therapies of the present invention. In one embodiment, the combination therapy is the use of angiopoietin-1 with a polypeptide form the tumor necrosis factor (“TNF”) family. In a preferred embodiment, the TNF polypeptide is TNFα. Other polypeptide inducers of apoptosis that may be used in the present invention include, but are not limited to, p53, Bax, Bak, Bcl-x, Bad, Bim, Bik, Bid, Harakiri, Ad E1B, Bad and ICE-CED3 proteases.

VIII. Pharmaceutical Compositions and Routes of Administration

Compositions of the present invention will have an effective amount of a gene for therapeutic administration in combination with an effective amount of a compound (second agent) that is an anti-cancer agent as exemplified above. Such compositions will generally be dissolved or dispersed in a pharmaceutically acceptable carrier or aqueous medium.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

The expression vectors and delivery vehicles of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. In a specific embodiment, the composition is injected into the tumor and/or in a regional administration. Such compositions would normally be administered as pharmaceutically acceptable compositions, described supra.

The vectors of the present invention are advantageously administered in the form of injectable compositions either as liquid solutions or suspensions; solid forms suitable for solution in, or suspension in, liquid prior to injection also may be prepared. These preparations also may be emulsified. A typical compositions for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All the essential materials and reagents required for inhibiting tumor cell proliferation may be assembled together in a kit. When the components of the kit are provided in one or more liquid solutions, the liquid solution preferably is an aqueous solution, with a sterile aqueous solution being particularly preferred.

For in vivo use, a chemotherapeutic agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the gene therapy and/or the chemotherapeutic drug.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

The active compounds of the present invention will often be formulated for parenteral administration, e.g., formulated for injection via the intravenous, intramuscular, subcutaneous, or even intraperitoneal routes. The preparation of an aqueous composition that contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Targeting of cancerous tissues may be accomplished in any one of a variety of ways. Plasmid vectors and retroviral vectors, adenovirus vectors, and other viral vectors all present means by which to target human cancers. The inventors anticipate particular success for the use of liposomes to target angiopoietin-1 genes to cancer cells. For example, DNA encoding angiopoietin-1 may be complexed with liposomes in the manner described above, and this DNA/liposome complex injected into patients with certain forms of cancer, such as breast cancer, intravenous injection can be used to direct the gene to all cell. Directly injecting the liposome complex into the proximity of a cancer can also provide for targeting of the complex with some forms of cancer. For example, cancers of the ovary can be targeted by injecting the liposome mixture directly into the peritoneal cavity of patients with ovarian cancer. Of course, the potential for liposomes that are selectively taken up by a population of cancerous cells exists, and such liposomes will also be useful for targeting the gene.

Those of skill in the art will recognize that the best treatment regimens for using angiopoietin-1 to suppress tumors can be straightforwardly determined. This is not a question of experimentation, but rather one of optimization, which is routinely conducted in the medical arts. The in vivo studies in nude mice provide a starting point from which to begin to optimize the dosage and delivery regimes. The frequency of injection will initially be once a wk, as was done some mice studies. However, this frequency might be optimally adjusted from one day to every two weeks to monthly, depending upon the results obtained from the initial clinical trials and the needs of a particular patient. Human dosage amounts can initially be determined by extrapolating from the amount of angiopoietin-1 used in mice. In certain embodiments it is envisioned that the dosage may vary from between about 1 μg angiopoietin-1 DNA/Kg body weight to about 5000 μg angiopoietin-1 DNA/Kg body weight; or from about 5 μg/Kg body weight to about 4000 μg/Kg body weight or from about 10 μg/Kg body weight to about 3000 μg/Kg body weight; or from about 50 μg/Kg body weight to about 2000 μg/Kg body weight; or from about 100 μg/Kg body weight to about 1000 μg/Kg body weight; or from about 150 μg/Kg body weight to about 500 μg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 μg/Kg body weight. In other embodiments, it is envisaged that higher does may be used, such doses may be in the range of about 5 mg angiopoietin-1 DNA/Kg body to about 20 mg angiopoietin-1 DNA/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

IX. Transgenic Animals/Knockout Animals

In one embodiment of the invention, transgenic animals are produced which contain a functional transgene encoding a functional angiopoietin-1 polypeptide or variants thereof. Transgenic animals expressing angiopoietin-1 transgenes, recombinant cell lines derived from such animals and transgenic embryos may be useful in methods for screening for and identifying agents that induce or repress function of angiopoietin-1. Transgenic animals of the present invention also can be used as models for studying indications such as cancers.

In one embodiment of the invention, an angiopoietin-1 transgene is introduced into a non-human host to produce a transgenic animal expressing a human or murine angiopoietin-1 gene. The transgenic animal is produced by the integration of the transgene into the genome in a manner that permits the expression of the transgene. Methods for producing transgenic animals are generally described by Wagner and Hoppe (U.S. Pat. No. 4,873,191; which is incorporated herein by reference), Brinster et al. 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

It may be desirable to replace the endogenous angiopoietin-1 by homologous recombination between the transgene and the endogenous gene; or the endogenous gene may be eliminated by deletion as in the preparation of “knock-out” animals. Typically, an angiopoietin-1 gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish. Within a particularly preferred embodiment, transgenic mice are generated which overexpress angiopoietin-1 or express a mutant form of the polypeptide. Alternatively, the absence of a angiopoietin-1 in “knock-out” mice permits the study of the effects that loss of angiopoietin-1 protein has on a cell in vivo. Knock-out mice also provide a model for the development of angiopoietin-1-related cancers.

As noted above, transgenic animals and cell lines derived from such animals may find use in certain testing experiments. In this regard, transgenic animals and cell lines capable of expressing wild-type or mutant angiopoietin-1 may be exposed to test substances. These test substances can be screened for the ability to enhance wild-type angiopoietin-1 expression and or function or impair the expression or function of mutant angiopoietin-1.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Materials and Methods

In a specific embodiment of the present invention, standard experimental procedures in the art are utilized. In a further specific embodiment, the following reagents and procedures are useful, in a particular embodiment, for experiments described in Examples 1 through 8.

Cell Lines. The HT29 human colon cancer cell line was obtained from the American Type Culture Collection (Manassas, Va.). Cells were cultured and maintained in minimal essential medium supplemented with 10% fetal bovine serum, 2 units/ml penicillin-streptomycin, vitamins, 1 mM sodium pyruvate, 2 mM L-glutamine, and nonessential amino acids at 37° C. in 5% CO₂ and 95% air (Dong et al., 1994). Cells were verified to be free of Mycoplasma, reovirus type 3, pneumonia virus of mice, mouse adenovirus, murine hepatitis virus, lymphocytic choriomeningitis virus, ectromelia virus, and lactate dehydrogenase virus (Microbiological Associates, Bethesda, Md.).

Subcloning of Ang-1 and Ang-2 into pcDNA3.1 and Transfection. The full-length cDNA for Ang-1 was a gift from Tona Gilmer, Ph.D. (Glaxo Wellcome Inc., Raleigh, N.C.), and the full-length cDNA for Ang-2 was a gift from Christopher D. Kontos, M.D. (Duke University, Durham, N.C.). These constructs were subcloned into the BamHI site of pcDNA3.1 (Invitrogen, San Diego, Calif.), a eukaryotic expression vector driven by the human cytomegalovirus promoter containing a hygromycin resistance gene. Subcloning into the BamHI restriction site yielded inserts in either the sense or antisense orientation; only inserts in the sense direction were used for the transfections. The orientation and completeness of the inserts were verified by restriction enzyme analyses and DNA sequencing (Core Sequencing Facilities, The University of Texas M. D. Anderson Cancer Center).

Vectors containing Ang-1 or Ang-2 or the vector alone (pcDNA3.1) were transfected into HT29 cells by lipofection according to the manufacturer's protocol (Boehringer Mannheim Co., South Africa). Selective medium containing 200 μg/ml of hygromycin was added 48 h later, and viable colonies were selected and expanded. Cells from subconfluent cultures were then harvested for northern blot analysis and in vivo animal experiments as described below.

Isolation of mRNA and Northern Blot Analysis. Northern blot analysis was performed as described elsewhere (Ellis et al., 1996). After prehybridization, the membranes were probed for Ang-1 or Ang-2 (with full-length cDNA probes) and glyceraldehyde-phosphate dehydrogenase (GAPDH, ATCC, Manassas, Va.) as an internal control. Each cDNA probe was purified by agarose gel electrophoresis, recovered with the QIAEX gel extraction kit (QIAGEN Inc., Chatsworth, Calif.), and radiolabeled by the random primer technique with a commercially available kit (Amersham Corp.). Nylon filters were washed at 65° C. with 30 mmol/l NaCl, 3 mmol/l sodium citrate (pH 7.2), and 0.1% sodium dodecyl sulfate. Autoradiography was then performed.

Animals and Tumor Cell Inoculation. Eight-week-old male nude mice were obtained from the National Cancer Institute's Animal Production Area (Frederick, Md.), acclimated for one week while caged in groups of five. Mice were fed a diet of animal chow and water ad libitum throughout the experiment. Mice were randomly assigned to one of four treatment groups (10 mice per group); body weight at assignment was no different among the groups. After cell viability was verified as being 80% or more with a trypan blue exclusion test, HT29 cells (1×10⁶ cells in 200 μl) were injected by means of a 30-gauge needle and a 1-ml syringe subcutaneously in the right flank of the animals. Tumor growth was measured every second to third day. Tumor volume was calculated as (diameter x length)/2. All animal studies were approved by the Institutional Animal Care and Use Committee (IACUC) of M. D. Anderson Cancer Center. Animals in all four groups were euthanized 3 weeks after tumor cell inoculation because of the large tumors that had appeared by that time in the Ang-2-transfected group. Tumors were harvested and placed in either 10% formalin for paraffin fixation or optimum cutting temperature (OCT) (Miles Inc., Elkhart, Ind.) solution and snap frozen.

Immunohistochemical Analyses. Antibodies for immunohistochemical analyses were obtained as follows: rat anti-mouse CD31/PECAM-1 antibody from Pharmingen (San Diego, Calif.); mouse anti-PCNA clone PC10 DAKO A/S from DAKO Corp. (Carpinteria, Calif.); goat anti-human angiopoietin-1 and -2 antibody from Santa Cruz Biotechnology (Santa Cruz, Calif.); peroxidase-conjugated goat anti-rat immunoglobulin (IgG) (H+L) and fluorescein-conjugated anti-goat IgG from Jackson Research Laboratories (West Grove, Pa.); peroxidase-conjugated rat anti-mouse IgG2a from Serotec Harlan Bioproducts for Science, Inc. (Indianapolis, Ind.); and Alexa 594 goat anti-rat IgG (H+L) from Molecular Probes (Eugene, Oreg.).

Paraffin-embedded tumors were sliced in 4- to 6-μm sections, mounted on positively charged superfrost slides (Fisher Scientific Co., Houston, Tex.), and allowed to dry overnight at room temperature. Sections were deparaffinized in xylene followed by 100%, 95%, and 80% ethanol washes and then rehydrated in phosphate-buffered saline (PBS), pH 7.5. These sections were used for hematoxylin and eosin (H & E) staining and detection of proliferating cell nuclear antigen (PCNA). Sections analyzed for PCNA were microwaved for 5 min to increase antigen retrieval.

Tumors that had been frozen in OCT solution were sectioned 8- to 10-μm in thickness, mounted on positively charged slides, and air-dried for 30 min. Tissue sections were then fixed in cold acetone (5 min), followed by 1:1 acetone/chloroform (5 min) and acetone (5 min), and then washed with PBS 3 times, with each wash lasting 3 min. Specimens were then incubated with 3% H₂O₂ in methanol for 12 min at room temperature to block endogenous peroxidase, washed 3 times (3 min each wash) with PBS (pH 7.5), and incubated for 20 min at room temperature in a protein-blocking solution consisting of PBS supplemented with 1% normal goat serum and 5% normal horse serum. The primary antibodies directed against CD31 and PCNA were diluted 1:200 and 1:50, respectively, in protein-blocking solution and applied to the sections, which were incubated overnight at 4° C. Sections were then rinsed 3 times (3 min each wash) in PBS and incubated for 10 min in protein-blocking solution before the addition of peroxidase-conjugated secondary antibody. The secondary antibodies used for CD31 (peroxidase conjugated goat anti-rat IgG) and PCNA (peroxidase conjugated rat anti-mouse IgG2a) staining were diluted 1:200 and 1:100, respectively, in protein-blocking solution. After incubating with the secondary antibody for 1 h at room temperature, the samples were washed and incubated with stable diaminobenzidine (DAB, Research Genetics, Huntsville, Ala.) substrate. Staining was monitored under a bright field microscope, and the reaction was stopped by washing with distilled water. Sections were counterstained with Gill's No. 3 hematoxylin (Sigma Chemical Co., St. Louis, Mo.) and mounted with Universal Mount (Research Genetics) for 15 s. Treatment procedures for control specimens were similar except that the primary antibody was omitted.

Immunofluorescent Staining for Angiopoietin, CD31, and TUNEL. Frozen sections were stained by immunofluorescence for Ang-1, Ang-2, and CD31 by immunofluorescence according to the same protocol as described above, with the following modifications. After sections were incubated overnight at 4° C. with the primary antibody (Angiopoietin, 1:100, Santa Cruz Biotechnology, Santa Cruz, Calif.), washed, and incubated with protein-blocking solution, they were incubated for 1 h at room temperature with a secondary antibody directed against Ang-1, Ang-2 (fluorescein-conjugated anti-goat IgG (Jackson Research Laboratories, West Grove, Pa.), or CD31 (Alexa 594 goat anti-rat IgG, Molecular Probes, Eugene, Oreg.).

Terminal dUTP nick-end label (TUNEL) staining was performed according to the manufacturer's protocol (Promega, Madison, Wis.). Briefly, the sections were fixed with 4% methanol-free paraformaldehyde, washed, permeabilized with 0.2% Triton X-100, washed, incubated with the kit's equilibration buffer, incubated with a reaction mix containing equilibration buffer, nucleotide mix, and the TdT enzyme at 37° C. for 1 h, incubated for 15 min at room temperature with 2×standard saline citrate to stop the TdT reaction, washed, and stained with 4,6-diamidino-2-phenylindole-2HCl (DAPI) (to visualize the nuclei), after which glass coverslips were applied.

Quantification of CD31 (Tumor Vessels) and PCNA (Tumor Cell Proliferation). Numbers of tumor vessels and PCNA-positive cells were counted by light microscopy in three random 0.159-mm² fields at 10×magnification with a Sony 3-chip camera (Sony, Montvale, N.J.) mounted on a Zeiss universal microscope (Carl Zeiss, Thornwood, N. Y.) and Optimas image analysis software (Bisoscan, Edmond, Wash.) installed in a Compaq computer with a Pentium chip, a frame grabber, an optical disk storage system, and a Sony color printer. Apoptosis was quantified by immunofluorescence by imaging sections digitally and processing them with Adobe Photoshop Software (Adobe Systems, Mountain View, Calif.) as follows. CD31-positive ECs were detected by localized red fluorescence with a rhodamine filter. Tumor cell (TC) and EC apoptosis was determined by localized green fluorescence (for TCs) or green with red fluorescence (for ECs) with a fluorescein filter. Nuclei were detected by blue fluorescence of the DAPI with its respective filter. Apoptotic cells were counted in five random 0.011-mm² fields per slide.

Cell Viability Assay. Two thousand cells were plated in 96-well plates. At 24, 48, and 72 hours the MTT (1-[4,5-Dimethylthiazol-2-yl]-3,5-diphenylformazan) assay was done as follows: 400 μl of 2.5 mg/ml solution of MTT was added to wells and incubated for 2 hours at 37° C. The supernatant was removed, and the reaction was stopped with dimethylsulfoxide, 100 μl/well. The plates were placed on a shaker for 1 minute, and the absorbance was determined on a plate reader at 570 γ. Each assay was repeated four times.

Statistical Analysis. Body weight, tumor volume, and numbers of CD31-positive and PCNA-positive cells were compared by using unpaired Student's t-tests (InStat for Macintosh, GraphPad software, San Diego, Calif.). Densitometric analysis was performed (Image Quant software, Molecular Dynamics, Sunnyvale, Calif.) to quantify the results of Northern blot analyses in the linear range of the film. GAPDH mRNA was used as an internal control for loading.

Tissue Specimens. Colon cancer specimens, uninvolved mucosa, and liver metastases from colon cancer and adjacent liver were obtained from the Surgical Pathology Suite at The University of Texas M. D. Anderson Cancer Center immediately after their resection. The specimens were either frozen in optimum cutting temperature (OCT) solution (Miles Inc., Elkhart, Ind.) and stored at −80° C. or snap-frozen in liquid nitrogen at the time of their collection. Specimens were obtained under a protocol approved by the institutional Clinical Research Committee.

Cell Lines. Human colon cancer cell lines were either obtained from the American Type Culture Collection (Manassas, Va.) (HT-29, NCIH747, LOVO, SW480, SW620, T84, SNUC2B, GEO, RKO), were the generous gifts of Dr. I. J. Fidler (The University of Texas M. D. Anderson Cancer Center) (KM12L4, KM12C, KM12SM, KM20, KM23), or Dr. Daniel J. Hicklin (ImClone Systems Inc., New York, N. Y.) (EC1022, CC415764, CC422867, CC421717). All cell lines were cultured and maintained in minimum essential medium supplemented with 10% fetal bovine serum (FBS), 2 units/ml penicillin-streptomycin, vitamins, 1 mM sodium pyruvate, 2 mM L-glutamine, and nonessential amino acids at 37° C. in 5% CO₂ and 95% air. (Tanaka et al., 1999) For mRNA extraction experiments, cells were harvested from subconfluent cultures. Cells were verified to be free of Mycoplasma, reovirus type 3, pneumonia virus of mice, mouse adenovirus, murine hepatitis virus, lymphocytic choriomeningitis virus, ectromelia virus, and lactate dehydrogenase virus (Microbiological Associates, Bethesda, Md.).

mRNA Extraction and Reverse Transcription-Polymerase Chain Reaction. Polyadenylated mRNA was extracted from 107 to 108 tumor cells growing in culture with the FastTrack mRNA isolation kit (Invitrogen Corp., San Diego, Calif.). Reverse transcription-polymerase chain reaction (RT-PCR) was performed as described by Witzenbichler et al. (1998) with the following modifications. Briefly, a 10-μl reaction mixture containing 1 μg mRNA and 20 μg/ml oligodeoxythymidine (Life Technologies Inc., Grand Island, N. Y.) was heated at 70° C. for 10 minutes. After the mixture cooled, 60 units of human placenta ribonuclease inhibitor (Promega, Madison, Wis.) and 200 units of Maloney's murine leukemia virus RNase II reverse transcriptase (Life Technologies, Inc.) were added in a final 20-μl reaction mixture containing 1 mmol/L each dNTP (Amersham, Arlington Heights, Ill.), 10 mmol/L dithiothreitol, 25 mmol/L Tris-HCl, pH_{—}8.3, 75 mmol/L KCl, and 3 mmol/L MgCl₂, incubated for 1 hour at 42° C., heated 5 minutes at 95° C., and diluted to 50 μl with double-distilled water. A 3-μl portion of the reaction mixture was subjected to PCR amplification in a 50-μl reaction mixture that contained 1 μmol/L of each of the two primers, 1.5 mmol/L MgCl₂, 0.2 mmol/L each of four deoxynucleotides, and 2.5 units Taq polymerase (Promega). PCR amplification of Ang-1, Ang-2, and Tie-2 was performed in 40 cycles of the following: 30 seconds of denaturing at 95° C., 5 seconds of annealing at 56° C., and 1 minute of extension at 72° C. PCR products were analyzed by electrophoresis of 10 μl of each PCR reaction mixture in a 1.5% agarose gel, and bands were visualized by ethidium bromide staining.

The primers chosen (Asahara et al., 1998) were as follows:

| | | |
|---|---|---|
| Tie-2: | sense 3′ ATCCCATTTGCAAAGCTTCTGGCTGGC 5′ | (SEQ ID NO:29) and antisense |
| | 5′TGTGAAGCGTCTCACAGGTCCAGGATG3′ | (SEQ ID NO:30) |
| Ang-1: | sense 3′ GGGGGAGGTTGGACTGTAAT 5′ | (SEQ ID NO:31) and antisense |
| | 5′AGGGCACATTTGCACATACA3′ | (SEQ ID NO:32); and |
| Ang-2: | sense 3′ GGATCTGGGGAGAGAGGAAC 5′ | (SEQ ID NO:33) and antisense |
| | 5′CTCTGCACCGAGTCATCGTA3′ | (SEQ ID NO:34). |
| | Human umbilical vein endothelial cell | |
| | RNA was used as a positive control. | |

Antibodies for Immunohistochemical Analyses. Antibodies for immunohistochemical analyses were obtained as follows: monoclonal anti-human CD31 from Pharmingen (San Diego, Calif.); goat anti-human angiopoietin-1 and -2 antibodies from Santa Cruz Biotechnology (Santa Cruz, Calif.); mouse anti-human smooth muscle actin from DAKO (Denmark); mouse anti-human cytokeratin-22 from Fisher Scientific Co. (Houston, Tex.); fluorescein-conjugated anti-goat IgG from Jackson Research Laboratories (West Grove, Pa.); and Alexa 594 goat anti-mouse from Molecular Probes (Eugene, Oreg.).

Immunohistochemical Staining for Ang-1, Ang-2, CD31, Cytokeratin-22, and Smooth Muscle Actin in Frozen Tissue Specimens. Tissue specimens frozen in OCT were sectioned (8-10 μm), mounted on positively charged Superfrost slides (Fisher Scientific Co.), and air-dried for 30 minutes. Snap-frozen tissues were fixed in cold acetone (5 minutes) followed by 1:1 acetone:chloroform (5 minutes) and acetone (5 minutes) and then washed with phosphate-buffered saline (PBS) 3 times for 3 minutes each time. All samples were incubated with 3% hydrogen peroxide in methanol for 12 minutes at room temperature to block endogenous peroxidase. Sections were then washed 3 times for 3 minutes each time with PBS (pH 7.5) and then incubated for 20 minutes at room temperature in a protein-blocking solution consisting of PBS supplemented with 1% normal goat serum and 5% normal horse serum. The primary antibodies directed against CD31 (diluted 1:400), smooth muscle actin (diluted 1:50), or cytokeratin-22 (Fisher Scientific Co.; no dilution) were applied to the sections and incubated overnight at 4° C. Sections were then rinsed 3 times for 3 minutes each time in PBS and incubated for 10 minutes in protein-blocking solution. In a darkened room, the blocking solution was drained, and the samples were incubated with a 1:200 dilution Alexa 594 goat anti-mouse secondary antibody (Molecular Probes) for 1 hour at room temperature. Samples were then washed 3 times with PBS and exposed to protein-blocking solution for 20 minutes. The second primary antibody, directed at either Ang-1 or Ang-2 at a dilution of 1:100, was then added for 3 hours at room temperature. Sections were then rinsed 3 times for 3 minutes each time in PBS and incubated for 10 minutes in protein-blocking solution. Once again, in a darkened room, the blocking solution was drained and the samples were incubated with a 1:200 dilution of fluorescein-conjugated anti-goat IgG antibody for 1 hour. The samples were then washed with PBS and mounted with coverslips. Immunofluorescence microscopy was conducted with a 40×objective (Zeiss Plan-Neofluar, Carl Zeiss Inc., Thornwood, N. Y.) on an epifluorescence microscope equipped with narrow bandpass excitation filters (Chroma Technology Corp., Brattleboro, Vt.) to individually select for green, red, and blue fluorescence. Images were captured with a Hamatsu 58-10 camera (Hamatsu Inc., Japan) mounted on a Zeiss Axioplan microscope (Carl Zeiss Inc.) using Optimas image analysis software (Media Cybernetics, Silver Spring, Md.). Images were further processed with Adobe Photoshop software (Adobe Systems, Mountain View, Calif.).The presence of CD31 (endothelial cells), cytokeratin-22 (epithelial cells), and smooth muscle actin (pericytes) was identified by red fluorescence, and angiopoietins were detected by green fluorescence.

Statistical Analysis. Comparisons of Ang-1 or Ang-2 expression in colon cancer specimens, cell lines, and normal mucosa were done with Chi-square analysis (Instat for Macintosh, GraphPad Software, San Diego, Calif.). A P value of 0.05 was deemed significant.

### Example 2

### ANG-1 AND ANG-2 Expression in Transfected Cell Cultures

Six clones each were isolated from the Ang-1, Ang-2-, and vector only transfected cell cultures. An isolated clone for Ang-1 transfectants and the pooled clones for Ang-2 transfectants demonstrated the highest expression of exogenous Ang-1 and Ang-2, respectively. By northern blot analysis parental HT29 colon cancer cells expressed endogenous Ang-2 but not Ang-1 (although, Ang-1 was expressed by RT-PCR). Based on densitometry readings, the Ang-2 pooled transfectants had 8 times greater exogenous Ang-2 production, when compared to endogenous Ang-2. The fold increase for Ang-1 could not be calculated due to the fact that endogenous Ang-1 was not detected in the parental cell line. Ang-1 had 10 times greater exogenous Ang-I production when compared to the other selected clones. The pooled empty vector transfectants were utilized for in vivo experiments.

### Example 3

### Tumor Growth and Body Weight in Experimental Animals

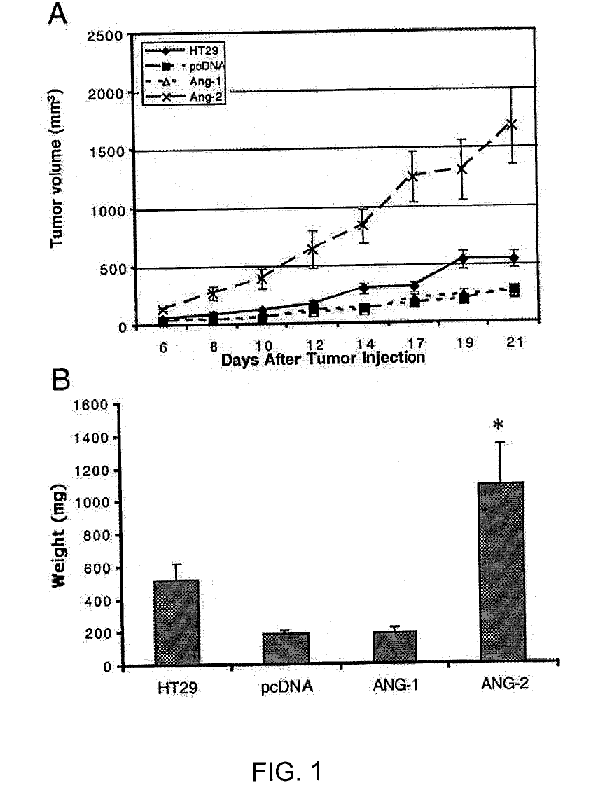

Body weight was no different between the treatment and control groups before or after the treatment. Tumors in the mice treated with the Ang-2-transfected HT29 cells were significantly larger (FIG. 1A) and heavier (FIG. 1B) than those in the other groups. All mice were killed on treatment-day 21 because the tumors in the Ang-2 group at that time were the maximum size allowed by the IACUC. Tumor volume was no different among the Ang-1-transfected, vector-only-transfected, and parental-cell groups. Growth rate based on clonal variation was assessed using the MTT assay. Growth rates of the Ang-1, Ang-2 and empty vector transfectants were not different.

### Example 4

### Effect of Transfection on Angiopoietin Expression and Tumor Vessel Counts

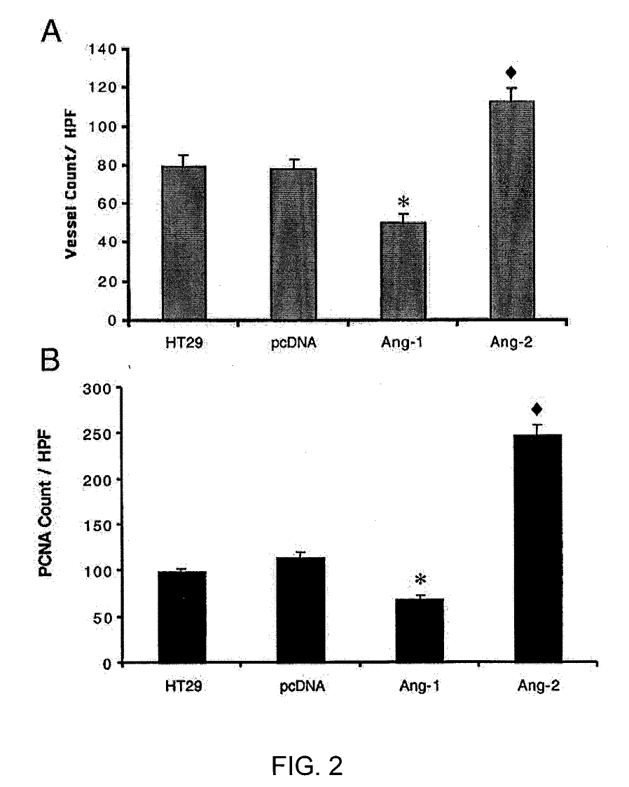

Immunohistochemical staining of harvested tumors confirmed high expression of Ang-1 in the group injected with the Ang-1-transfected cells and high expression of Ang-2 in the group injected with the Ang-2-transfected cells relative to the expression of these compounds in the groups injected with the HT29 parental cell line or the pcDNA transfectants. Immunohistochemical staining for CD31 revealed significantly more tumor vessels in the Ang-2-transfected group, and significantly fewer tumor vessels in the Ang-1-transfected group, than in the other groups (P<0.05) (FIG. 2A).

### Example 5

### Effect of Overexpression of Ang-1/-2 on PCNA Expression, Endothelial Cell and Tumor Cell Apoptosis

Immunohistochemical staining for PCNA (a measure of tumor cell proliferation) revealed that the number of PCNA-positive cells was greatest in the tumors from the Ang-2-transfected group, and least in tumors from the Ang-1-transfected group, relative to those in the other groups (P<0.05) (FIG. 2B). Immunofluorescent TUNEL staining of tumor sections from the four groups revealed slightly more TUNEL-positive cells in the tumors from the Ang-2 group than in the tumors from the other three groups. However, in comparing these slides with the H&E slides, it became apparent that the large number of TUNEL-positive cells in the Ang-2 group was due to necrosis rather than apoptosis, since the tumors in the Ang-2 group were quite large and necrotic when they were harvested. Double staining (TUNEL and CD31) revealed no difference in EC apoptosis among the four groups.

### Example 6

### Ang-1, Ang-2 and Tie-2 Expression in Colon Cancer Cell Lines

Eighteen colon cancer cell lines were examined by RT-PCR for the expression of Ang-1, Ang-2, and their receptor, Tie-2. As expected, none of the colon cancer cell lines expressed the tyrosine kinase receptor Tie-2; 7 colon cancer cell lines (39%) expressed Ang-1 and 14 colon cancer cell lines (78%) expressed Ang-2 (P<0.05). RT-PCR analyses of 11 human colon cancer specimens and 9 specimens of human colon mucosa revealed that 6 (54%) of the colon cancer specimens expressed Ang-1, whereas all 11 (100%) of the colon cancer specimens produced Ang-2 (P<0.05). In contrast, Ang-1 was expressed in 7 (78%) of the 9 normal colon mucosa specimens and Ang-2 was expressed in 5 (56%) of those specimens (p=0.62) (Table 1).

Immunofluorescent staining was performed on 20 colon cancer specimens and adjacent uninvolved mucosa, and on 5 colon cancer liver metastases with adjacent uninvolved liver. Ang-1 was found in normal colon mucosa and in normal liver parenchyma in all cases studied. In contrast, Ang-1 production was barely or not detectable in colon cancer specimens and was not detected in liver metastases. Ang-2 was found in both normal colon mucosa and cancer and in both normal liver parenchyma and liver metastases in all specimens. Double-staining for the angiopoietins and CD31 (endothelial cell marker) revealed that Ang-1 and Ang-2 were present in periendothelial cells and that Ang-2—but not Ang-1—was present in tumor cells.

To more accurately localize the origin of the angiopoietins, 5 representative samples were double-stained for Ang-1 or Ang-2 and cytokeratin-22 (an epithelial cell marker) or smooth muscle actin (a pericyte marker). Ang-1 was found to be expressed in normal colon mucosa and pericytes. Ang-2 was also detected in normal colon mucosa and pericytes, but unlike Ang-1, Ang-2 was also detected in colon cancer epithelium.

### Example 7

### Overexpression of Ang-1 Reduces Tumor Growth and Angiogenesis of Colon Cancer Hepatic Metastases

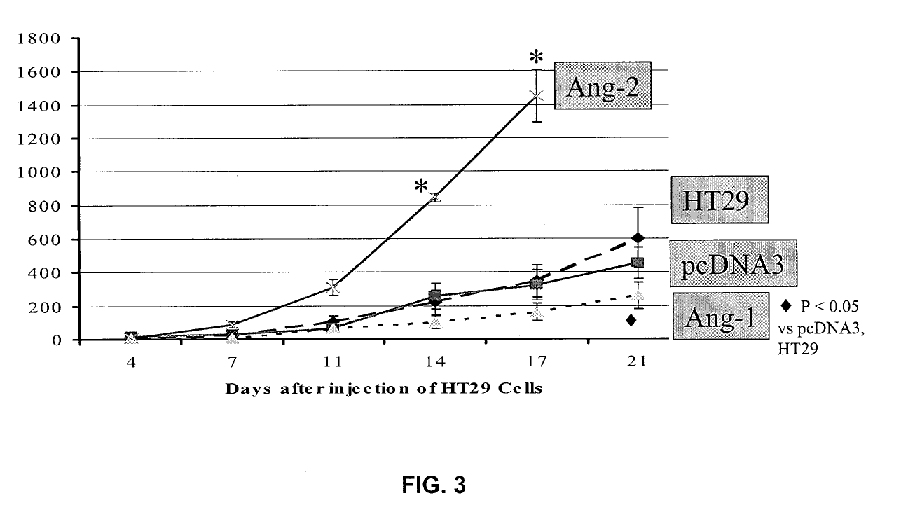

First, FIG. 3 illustrates the effect of overexpression of Ang-1 and Ang-2 on SQ tumor growth, wherein HT29 colon cancer cells were transfected. As early as day 11, it is clear that Ang-2 accelerates tumor growth, whereas by day 14 Ang-1 is leading to relative tumor dormancy.

For further experiments, athymic nude mice were utilized for a liver metastases model. HT29-Ang-1 or HT20-pcDNA3.1 (−) Hygro cells (1×10⁶) were injected into the liver and measurements were taken at 37 days when the control mice became moribund. The liver tumor incidence was 9/9 (100%) for pcDNA mice and 5/7 (71%) for Ang-1 mice.

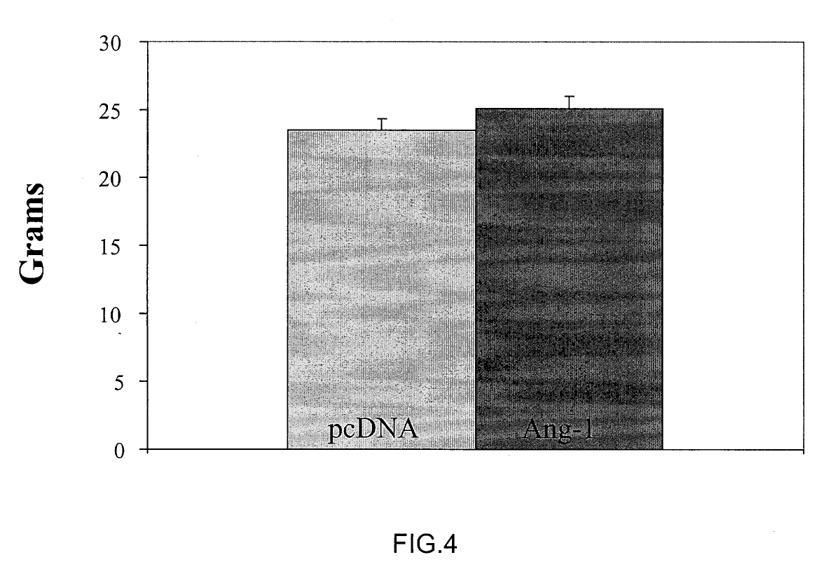

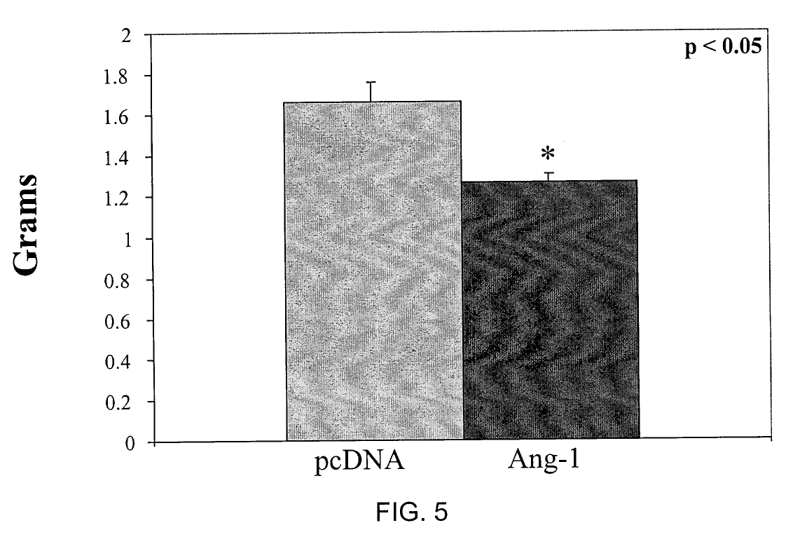

FIG. 4 shows final mouse weight after 37 days of liver tumor growth. Although mice in the control group became moribund, mice in the Ang-1 group were vital, however final mouse weight did not significantly differ compared to controls (p>0.05). FIG. 5 shows Ang-1 overexpression reduces hepatic colon cancer tumor growth. Mice were sacrificed and weights of excised livers were determined. Liver weights in the Ang-1 group were significantly lower compared to controls, reflecting a reduced extent of tumor burden (p<0.05; two-tailed Mann-Whitney Test).

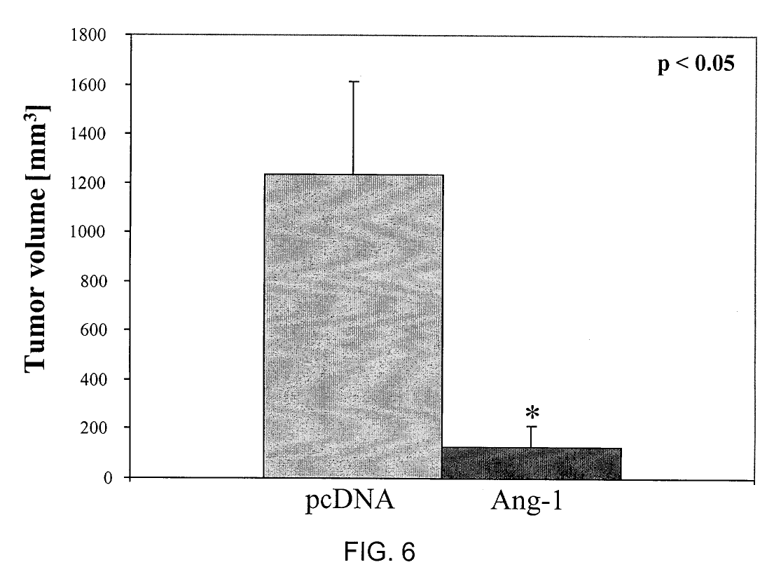

FIG. 6 shows the effect of Ang-1 overexpression on liver tumor volume. Livers were excised and diameters of liver tumors were determined. Tumor volumes were calculated using the formula: [W²]×L×0.5. Ang-1 overexpressing HT29 tumors had significantly lower tumor volume compared to control (pcDNA) (p<0.05; two-tailed Mann-Whitney Test).

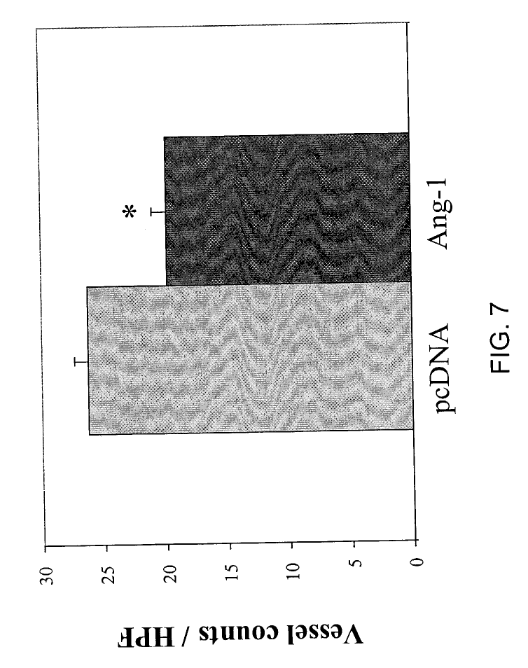

FIG. 7 demonstrates the effect of Ang-1 overexpression on microvessel density in colorectal liver tumors. Frozen tumor sections were stained for CD31 and microvessels were counted in four different quadrants of the liver tumors (2 mm inside liver/tumor interface). Ang-1 overexpressing tumors had significantly lower vessel counts compared to control (pcDNA) (p<0.03) (two-tailed Student-t Test).

Livers comprising colon cancer liver tumors were analyzed, and the in vivo effect of Ang-1 overexpression on tumor growth of liver implanted HT29 colon cancer cells was determined. Mice were sacrificed when controls (pcDNA) became moribund and livers were excised. Livers from mice comprising Ang-1 overexpression showed reduced tumors compared to livers from control mice.

### Example 8

### Overexpression of Ang-1 Prevents Ascites Formation in a Peritoneal Carcinomatosis Model of Colorectal Cancer By Reducing Vascular Permeability

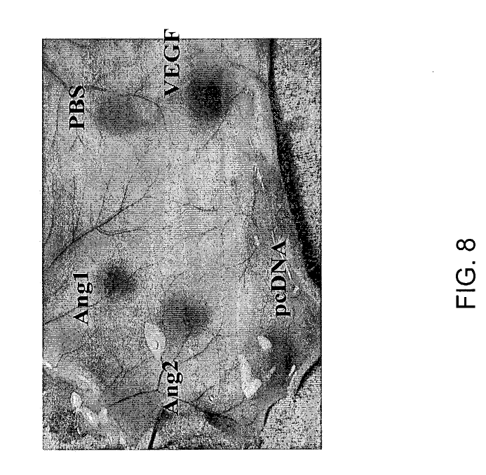

FIG. 8 demonstrates an intradermal Miles assay demonstrating the effect of angiopoietins on vascular permeability. Nude mice were i.v. injected with Evans dye blue (0.5%). After 10 min circulation time, 50 μL of conditioned media from Ang-1, Ang-2 or pcDNA transfected HT29 cells was injected i.e. Mice were sacrificed 20 min thereafter and images of the subcutis were obtained. VEGF (10 ng/mL) served as positive control for vascular permeability.

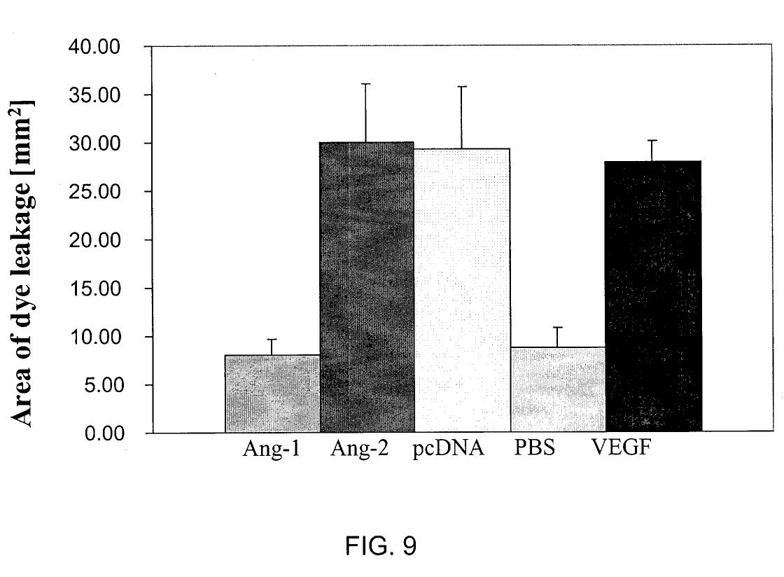

FIG. 9 shows the effect of angiopoietins on vascular permeability determined by area of leakage. Leakage areas were calculated in all mice (n=4) for each injection site (a×b×π) and averages were obtained. Ang-1 abrogated vascular leakage induced by conditioned media, compared to Ang-2 and pcDNA CM.

Ultrastructural analysis of vessels of Ang-1 vs. Ang-2 or pcDNA transfected HT29 tumors illustrates an implication for vascular permeability. Upon electron microscopy analysis, there is reduced pericyte coverage in Ang-2 expressing tumors. There is also reduced VVO formation in Ang-2 expressing tumors compared to Ang-1 or pcDNA, suggesting Ang-2 increases permeability. Similar VVOs were observed for Ang-1 and pcDNA tumors. Increased septal structure formation in Ang-2 tumors was also observed, suggesting an increasein angiogensis. There was no effect of angiopoietins on inter-endothelial cell junctions.

### Example 9

### Additional Experiments

Pericyte coverage (IHC) of Ang-1 and pcDNA tumor vessels in HT29 liver tumors is determined. Standard methods in the art are utilized.

Cell adhesion and migration assay (pcDNA vs. Ang-1) is performed. Standard methods in the art are utilized.

### Example 10

### Additional Exemplary Materials and Methods

In a specific embodiment of the present invention, standard experimental procedures in the art are utilized. In a further specific embodiment, the following reagents and procedures are useful, in a particular embodiment, for experiments described in the following Examples.

Cell Culture. The human colon cancer cell line HT29 and HUVECs were purchased from the American Type Culture Collection (Manassas, Va.). HT29 cells were maintained in MEM supplemented with 10% FBS, 2 units/ml penicillin-streptomycin mixture (Flow Laboratories, Rockville, Md.), a 2×vitamin solution (Life Technologies, Inc., Grand Island, N. Y.), 1 mM sodium pyruvate, 2 mM L-glutamine, and nonessential amino acids and incubated in 5% CO₂—95% air at 37° C., as described previously (Ahmad et al., 2001). HUVECs were cultured in MEM supplemented with 15% FBS and basic fibroblast growth factor as previously described (Liu et al., 2001).

Stable Transfection. The full-length cDNA for Ang-1 was obtained from Dr. T. Gilmer (GlaxoSmithKline, Research Triangle Park, N.C.). The construct was subcloned into a pcDNA3.1 vector (InVitrogen, Carlsbad, Calif.) containing a hygromycin resistance gene. The vector containing Ang-1 or the empty pcDNA vector was transfected into HT29 cells with lipofectin according to the manufacturer's protocol (Boehringer Mannheim Co., Randburg, South Africa) (Ahmad et al., 2001). Cells were thereafter grown in selective media (10% FBS-MEM containing 200 ng/ml hygromycin). Cell clones were subsequently screened by northern blot analysis for an increase in Ang-1 mRNA expression relative to that in the pcDNA-transfected cells, as previously described (Ahmad et al., 2001). For in vivo experiments, HT29 cells that had been transfected with Ang-1 or pcDNA were harvested from subconfluent cultures by rinsing with PBS and trypsinizing (0.25% trypsin and 0.02% EDTA) for 3 min. Cells were washed in 10% FBS-MEM and counted. Cell viability was assessed by trypan blue exclusion, verifying that cell viability was >90% in both cell lines. Cells were then centrifuged and resuspended in Hanks' balanced salt solution for injection into mice.

Quantification of VEGF Protein in Conditioned Medium from Transfected Colon Cancer Cells. VEGF protein concentrations in conditioned media from Ang-1 or pcDNA-transfected HT29 cells were determined using an ELISA kit for human VEGF (Biosource International, Camarillo, Calif.). Cell culture supernatants (3 ml) from cells were collected after a 48 h incubation period (in 10% FBS-MEM (FBS does not contain detectable human VEGF)). Supernatants were subsequently collected after centrifugation for 5 min at 350 g. In parallel, cells in culture flasks were rinsed with PBS, trypsinized as described above, resuspended in 10% FBS-MEM and counted for each cell line. VEGF ELISA of 10-fold diluted conditioned media (due to excessive high VEGF levels) was performed according to the manufacturers protocol.

Animals. Eight-week-old male athymic nude mice or BALB/c mice (both obtained from the Animal Production Area of the National Cancer Institute and Development Center, Frederick, Md.) were acclimated for 1-2 weeks while caged in groups of five. Mice were housed as previously described (Bruns et al., 2000; Shaheen et al., 2001) and fed a diet of animal chow and water ad libitum throughout the experiment.

Colon Cancer Liver Tumor Model. To determine the effects of Ang-1 transfection on hepatic tumor growth of human colon cancer cells, Ang-1-transfected or pcDNA-transfected (control) cells (1×10⁶ cells in 50 μl injection volume) were directly injected into the livers of athymic nude mice after they had been randomly assigned to one of the two groups (7-9 mice per group). Body weight was similar between the groups at the beginning of the experiment. Mice were observed daily, and all mice were killed (when three in any one group showed decreased mobility or discomfort) by cervical dislocation after anesthesia induction with pentobarbital (Nembutal) (50 mg/kg). Body weights were measured, livers were excised, and liver weights and tumor diameters were subsequently determined. Tumor volumes were calculated with the equation width²×length×0.5. Tumor tissue was then harvested and either placed in 10% formalin for paraffin embedding or snap-frozen in OCT solution (Miles Inc., Elkhart, Ind.) in preparation for subsequent immunohistochemical analyses.

To confirm the paracrine effect of Ang-1 on in vivo angiogenesis, Ang-1 and pcDNA cells were mixed at various ratios (100% Ang-1:0% pcDNA, 50% Ang-1:50% pcDNA, 10% Ang-1:90% pcDNA, and 0% Ang-1:100% pcDNA) and injected into the liver as described above. Mice were observed and sacrificed according to the criteria described above. Tumor bearing livers were excised, their weights determined and tumor diameters measured.

Gelfoam In Vivo Angiogenesis Assay. Effects of Ang-1 on angiogenesis were investigated in a Gelfoam in vivo angiogenesis assay using male BALB/c mice (5 per group). Recombinant human Ang-1 (Ang-1 TFD (clustered Ang-1 is required for activation of Tie-2)) was the generous gift of Jocelyn Holash, Ph.D., Regeneron Pharmaceuticals (Tarrytown, N. Y.). The structure and clustering of Ang-1 TFD was recently further characterized by Davis et al. (2003). Sterile absorbable sponges (Pharmacia, Peapack, N.J.) were cut into 5×5×7 mm pieces and hydrated overnight at 4° C. in sterile PBS. Excess PBS was then drained by blotting onto sterile filter paper. The sponges were then soaked with 0.4% agarose (100 μl) containing either PBS (control) or Ang-1 TFD (1 μg/μl). The agarose-Gelfoam plugs were then allowed to harden for 1 h at room temperature before their subcutaneous implantation into BALB/c mice (5 mice per group). Mice were anesthetized with Nembutal (50 mg/kg) and the plugs were implanted subcutaneously via a midline incision of the abdominal skin and placed either towards the right flank (Ang-1 TFD plugs) or the left flank (PBS plugs). Fourteen days later, mice were sacrificed as described above (McCarty et al., 2002). Gelfoam plugs were harvested, placed in OCT solution, and snap-frozen in liquid nitrogen for subsequent immunohistochemical analyses.

Immunohistochemical Analyses of Tumor Vessel Density. Rat anti-mouse CD31/PECAM-1 antibody was obtained from PharMingen (San Diego, Calif.) and peroxidase-conjugated goat anti-rat IgG from Jackson Research Laboratories (West Grove, Pa.). Tumors that had been frozen in OCT were sectioned in 8-μm slices, mounted on positively charged slides, and air-dried for 30 min. Tissue sections were then fixed in cold acetone followed by 1:1 acetone/chloroform and acetone and then washed with PBS. Specimens were then incubated with 3% H₂O₂ in methanol for 12 min at room temperature to block endogenous peroxidase, washed three times with PBS (pH 7.5), and incubated for 20 min at room temperature in a protein-blocking solution consisting of PBS supplemented with 1% normal goat serum and 5% normal horse serum. The primary antibody directed against CD31 was diluted 1:800 in protein-blocking solution and applied to the sections, which were incubated overnight at 4° C. Sections were then rinsed in PBS and incubated for 10 min in protein-blocking solution before the addition of peroxidase-conjugated secondary antibody. The secondary antibody used for CD31 (peroxidase-conjugated goat anti-rat IgG) staining was diluted 1:200 in protein-blocking solution. After incubating with the secondary antibody for 1 h at room temperature, the samples were washed and incubated with stable diaminobenzidine (Research Genetics, Huntsville, Ala.) substrate. Staining was monitored under a bright-field microscope, and the reaction was stopped by washing with distilled water. Sections were counterstained with Gill's No. 3 hematoxylin (Sigma Chemical Co., St. Louis, Mo.) and mounted with Universal Mount (Research Genetics). CD31-stained vessels were counted (at 50× magnification) at four different quadrants of each tumor (2 mm inside the tumor-normal tissue interface) and averages were calculated. For all immunohistochemical studies, the primary antibody was omitted as a negative control.

Immunohistochemical Analyses of Tumor Cell Proliferation. Paraffin-embedded tissues were sectioned and stained for PCNA by using the mouse anti-PCNA clone PC10 DAKO A/S from DAKO Corp. (Carpinteria, Calif.). Paraffin-embedded tissues were sectioned in 4- to 6-μm slices, mounted on positively charged Superfrost slides (Fisher Scientific, Co., Houston, Tex.), and dried overnight. Sections were deparaffinized in xylene, followed by treatment with a graded series of alcohol washes (100%, 95%, 80% ethanol/ddH₂O (v/v)), rehydration in PBS (pH 7.5), and microwaving for 5 min for “antigen retrieval.” Immunohistochemical procedures were performed as described previously (Bruns et al., 2000). Positive reactions were visualized by incubating the slides with stable diaminobenzidine for 10-20 min. The sections were rinsed with distilled water, counterstained with Gill's hematoxylin for 1 min, and mounted with Universal Mount (Research Genetics). Slides were also stained with H&E to study overall tissue structure. The numbers of PCNA-positive and PCNA-negative tumor cells were determined in four random fields per tumor (at 100× magnification), and the percentage of PCNA-positive cells was then calculated.

Immunofluorescent Analyses of Pericyte-Covered Tumor Vessels and Ang-1 Expression in Hepatic Tumors. To determine pericyte coverage of tumor vessels in pcDNA-transfected and Ang-1-transfected liver tumors, double-staining for CD31 and α-smooth muscle actin (α-SMA) (DAKO Corp.) was performed according to a modified protocol as described elsewhere (Eberhad et al., 2000). Frozen sections of hepatic tumors were stained overnight (4° C.) for CD31/PECAM-1 (PharMingen) after acetone fixation as described above. Slides were rinsed with PBS (3 times for 3 min each time) and incubated for 10 min in protein-blocking solution before the addition of Texas Red-conjugated goat anti-rat secondary antibody (1:200) (Jackson ImmunoResearch Laboratories) and subsequent incubation for 1 h at room temperature under light protection. Antibodies were washed off with PBS (3 washes for 3 min each) and slides were blocked with nonspecific goat anti-mouse IgG Fab fragment (Jackson ImmunoResearch Laboratories), diluted 1:10 in protein block, for 1 h at room temperature to reduce background staining for the subsequent double-staining procedure. After another rinsing cycle with PBS (3 times for 3 min each time), slides were incubated for 10 min in protein-blocking solution. For pericyte staining (pericytes were defined as α-SMA positive cells in direct contact with endothelial cells), tumor sections were incubated overnight (4° C.) with mouse anti-α-SMA (DAKO) (1:2000 in protein block solution). The antibody was then rinsed off with PBS and protein-blocking solution was applied for 10 min. Alexa 488 (green) (Jackson ImmunoResearch Laboratories) rabbit anti-mouse secondary antibody (1:200 in protein-block) was added for 1 h at room temperature. Slides were rinsed in PBS and nuclei were stained with Hoechst dye (1:2000) for 2 min. Slides were analyzed with an epifluorescence microscope equipped with narrow bandpass excitation filters (Chroma Technology Corp., Brattleboro, Vt.) to individually select for green, red, and blue fluorescence. Images were captured with a C5810 Hamamatsu camera (Hamamatsu Photonics K.K., Bridgewater, N.J.) mounted on a Zeiss Axioplan microscope (Carl Zeiss Inc., Oberkochen, Germany) using Optimas image analysis software (Media Cybernetics, Silver Spring, Md.). Images were further processed with Adobe Photoshop software (Adobe Systems, Mountain View, Calif.). Double-stained slides were analyzed at 200× magnification for the degree of pericyte/CD31 co-localization as described elsewhere (Eberhard et al., 2000). The degree of pericyte coverage was evaluated in four fields per tumor (2 mm inside the tumor-normal tissue interface) and rated as either absent or full coverage (defined as covering >90% of the vessel). The average percentage of covered vessels relative to uncovered vessels was then calculated for each tumor.

To confirm that Ang-1 was overexpressed in hepatic tumors, immunohistochemistry detection of Ang-1 was analyzed using goat anti-Ang-1 antibody N-18 (Santa Cruz Biotechnology, Santa Cruz, Calif.). Frozen tumor sections were fixed and blocked with protein block solution as described above. The primary antibody was added at 1:100 dilution and incubated overnight at 4° C. Slides were processed and analyzed as described, except that Alexa 488 anti-goat (1:500) was added as secondary antibody for one hour.

Immunofluorescent Analyses of Vessel Density in Gelfoam Plugs. Frozen sections of the agarose-Gelfoam plugs were prepared and stained for CD31/PECAM-1 (PharMingen, San Diego, Calif.) as described above. For immunofluorescent analysis, slides were incubated with Texas Red-conjugated goat anti-rat secondary antibody (1:200) as described above. Vessels were counted under an epifluorescence microscope at four different “hot spots” in each Gelfoam plug at 50× magnification as described in the previous paragraph.

In Vivo Miles Permeability Assay. To investigate the effects of Ang-1 overexpression by tumor cells on vascular permeability, an intradermal Miles assay was performed. CM from Ang-1-transfected or pcDNA-transfected HT29 cells was collected after a 48-h incubation in 1% FBS-modified Eagle's medium at 80% cell density, centrifuged for 5 min at 350 g, and filtered through a 0.22-μm filter (Coming Inc., Corning, N. Y.). Nude mice (n=4) were injected intravenously with sterile 0.5% Evans blue dye (200 μl) via the tail vein. Ten minutes later, mice were given intradermal injections into the dorsal skin at three different sites-one for CM-Ang-1, one for CM-pcDNA, and one for VEGF (10 ng/ml) (R&D Systems Inc., Minneapolis, Minn.). The VEGF served as positive control for increased vascular permeability. The intradermal injections (50 μl per injection) were done with a 30-gauge needle. Mice were killed 20 min after the intradermal injections by cervical dislocation after anesthesia had been induced with Nembutal. The dorsal skin of each mouse was harvested to permit visualization of intradermal dye leakage. To determine the relative degree of vascular permeability, two dimensions (a and b) of the elliptically appearing area of dye leakage were obtained at each injection site by an observer blinded to the experimental group, and the area was calculated with the formula a×b×π.

Densitometric Quantification of Vascular Permeability. Densitometric analysis was performed using the NIH Image Analysis software (V1.62) from the National Institute of Health (Bethesda, Md.) as another means of quantifying the extent of dye leakage at the intradermal injection sites in each mouse. Digitally obtained images of the underside of the dorsal skin, including all injection sites, were converted to a gray-scale image, and dye density was analyzed at each site (threshold was set individually for each dorsal skin flap, but was constant for each mouse).

Endothelial Cell Co-culture and Tie-2 Phosphorylation assay. In order to verify functional overexpression of Ang-1 in transfected HT29 cells, Ang-1 and pcDNA transfected HT29 cells were co-cultured with HUVECs for 48 h using transwell culture dishes (Coming Inc., Corning, N. Y.). EC were thereafter harvested in PBS and protein was isolated for Tie-2 immunoprecipitation as described (Hawighorst et al., 2002). Briefly, 600 μg protein for each experiment was used for immunoprecipitation using rabbit Tie-2 antibody (sc-324) and A/G plus agarose (both from Santa Cruz Biotechnology). Protein was then separated on a denaturating 7.5% SDS-polyacrylamide gel for Western blot analysis of Tie-2 phosphorylation. Membranes were probed with mouse anti-phosphotyrosine (Upstate Biotechnology, Lake Placid, N. Y.) and Tie-2 phosphorylation levels were analyzed by densitometry. Membranes were additionally probed for Tie-2 to assure equal loading.

Statistical Analyses. All statistical analyses were done using InStat Statistical Software (V2.03, GraphPad Software, San Diego, Calif.), with P values of less than 0.05 considered to be statistically significant. Results of in vivo experiments were also tested for significant outliers using the Grubb's test for assessing outliers. Tumor-associated variables were tested for statistical significance using the two-tailed Student's t-test or the Mann-Whitney U test (for nonparametric data) as specified in the figure legends. Fisher's test was applied for comparing the incidence of hepatic tumor formation.

### Example 11

### Effect of Ang-1 Overexpression on Hepatic Tumor Growth.

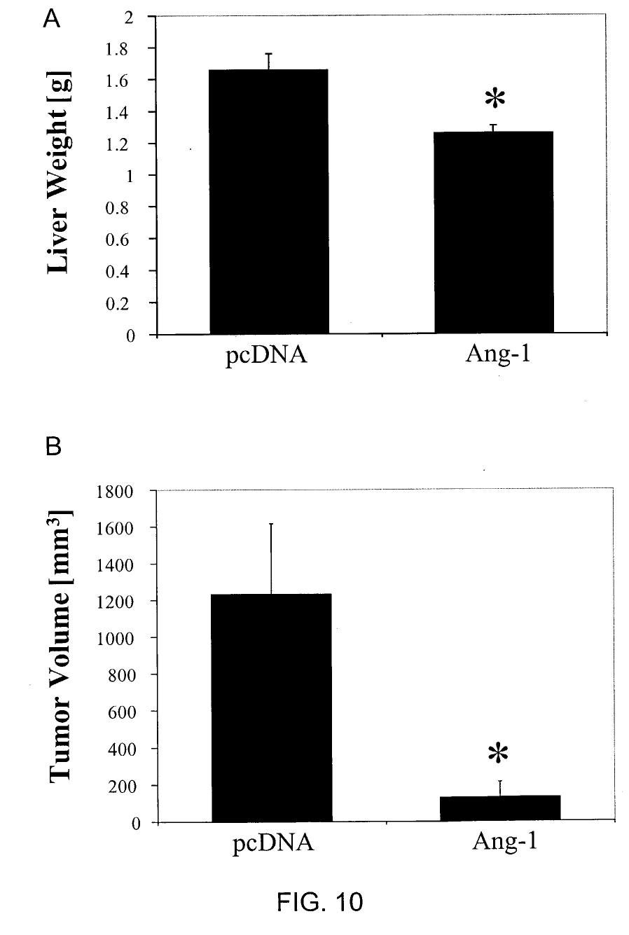

To evaluate the effects of Ang-1 overexpression on tumor growth of human colorectal cancer cells at the most common metastatic site (the liver), Ang-1-transfected or pcDNA-transfected (control) HT29 cells were injected directly into the liver parenchyma of mice to form single hepatic tumors. Mice with visible tumor spillage at the time of the injection were excluded from further analysis. The experiment was terminated after 37 days of tumor growth, when mice in the control group became moribund. All 9 mice in the control group and 71% (5 of 7) of the mice in the Ang-1 group developed liver tumors (P=0.17). However, Ang-1 overexpression led to a marked reduction of hepatic tumor burden (liver weight) (P<0.05) (FIG. 10A). Ang-1 overexpression in tumors also led to a significant decrease in tumor volume (P<0.05) (FIG. 10B). Images of excised livers were examined. All 9 of the mice in the pcDNA group and 5 of the 7 mice in the Ang-1 group developed liver tumors. Hepatic tumor volumes in the Ang-1 group were significantly smaller than those in the pcDNA control group (P<0.05). To confirm that Ang-1 secretion from the experimental cell line had no direct effect on tumor cell proliferation, the MTT assay was performed as previously described (Reinmuth et al., 2002). Growth rates of both Ang-1- and pcDNA-transfected cells were similar over 24 h and 48 h.

### Example 12

### Effect of Ang-1 Overexpression on Tumor Vessel Density and Tumor Cell Proliferation

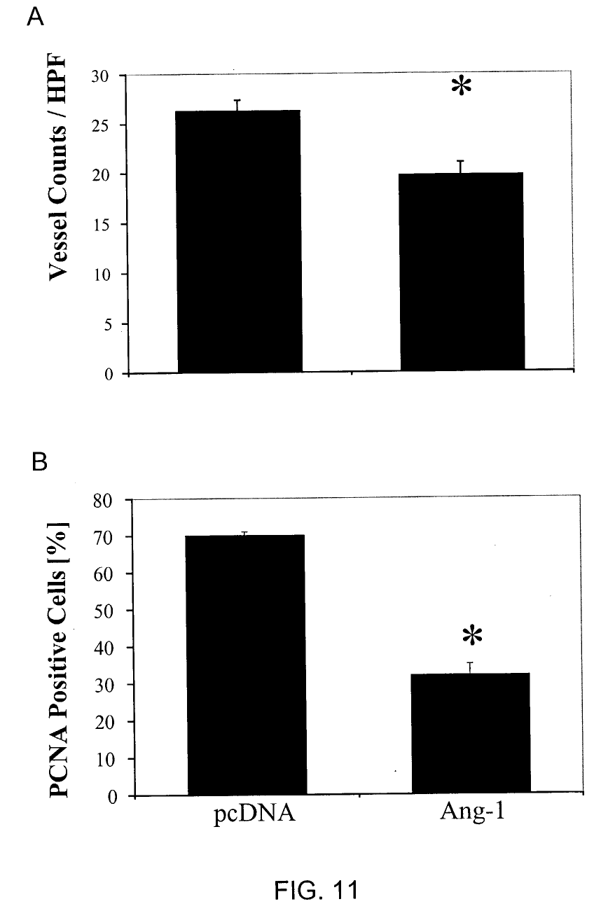

Tumor sections were stained for CD31 (PECAM-1) to allow vessels to be counted. Microvessel density was significantly reduced in Ang-1-expressing tumors (P<0.03) as compared with pcDNA tumors (FIG. 11A). To investigate whether Ang-1 overexpression has indirect effects on tumor cell proliferation (HT29 cells are Tie-2-negative by reverse transcriptase-polymerase chain reaction, data not shown), the percentage of proliferating tumor cells was evaluated by immunohistochemical staining for PCNA. Ang-1-overexpressing colon cancer tumors demonstrated significantly lower percentages of proliferating (PCNA-positive) tumor cells than did tumors in the pcDNA group (P<0.01) (FIG. 11B).

### Example 13

### Expression of Ang-1 and Effect of Ang-1 on Pericyte Coverage of Tumor Vessels

Immunohistochemical analysis of Ang-1 expression in hepatic tumors confirmed Ang-1 expression in HT29-Ang-1 tumors and normal tissues. In contrast, and consistent with previous studies (Ahmad et al., 2001), Ang-1 was not detected in mock transfected cells. The effect of Ang-1 on tumor endothelial cell pericyte coverage was investigated by immunofluorescent double staining of tumor vessels (CD31; red) and pericytes (α-SMA; green cells adjacent to ECs). Tumor sections were double-stained for CD31 (red) and α-smooth muscle actin (α-SMA, green) to evaluate the degree of pericyte coverage of tumor vessels. Tumor vessels in the Ang-1-transfected tumors seemed to be stabilized by increased pericyte association and higher extent of coverage as compared with lesser coverage in the pcDNA tumors.

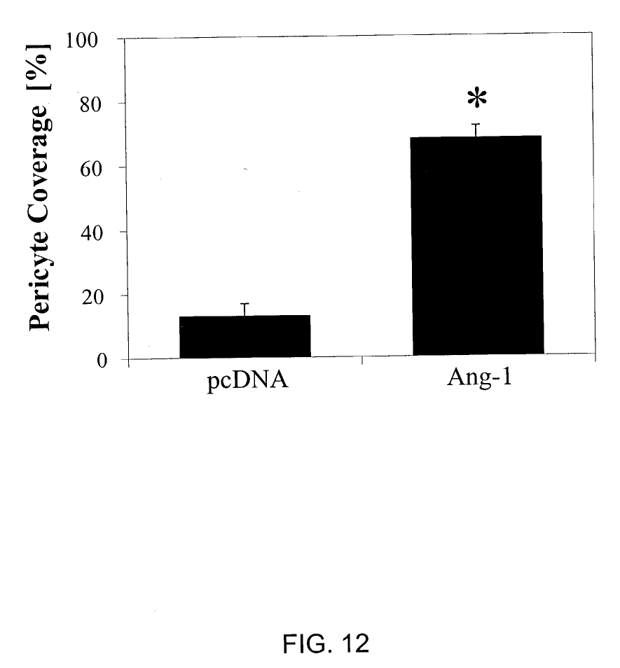

Ang-1 overexpression in HT29 hepatic tumors significantly increased the degree of pericyte coverage in the Ang-1 group (P<0.01) as compared with pcDNA tumors. In quantitative terms, 68% of tumor vessels in the Ang-1-transfected tumors were tightly associated with and surrounded by pericytes, but only 13% of tumor vessels in the pcDNA tumors showed the same degree of high coverage (FIG. 12).

### Example 14

### Paracrine Effect of Ang-1 Secretion on in Vivo Tumor Growth

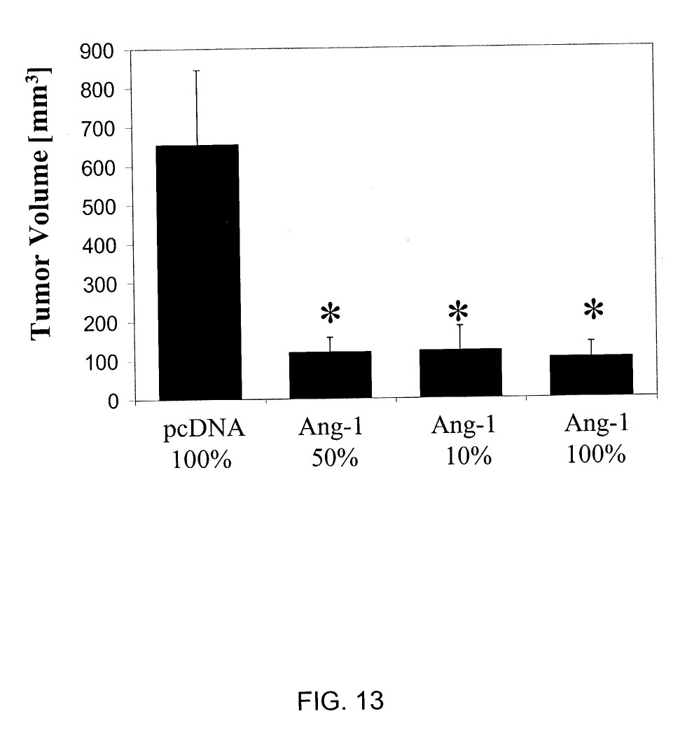

To confirm the paracrine effect of Ang-1 on in vivo angiogenesis, Ang-1 and pcDNA cells were mixed at various ratios and injected into the liver as described above. After 35 days of tumor growth, mice in the 100% pcDNA-group became moribund and the experiment was terminated. Tumor cell injections in this study revealed large tumor masses in the control group, but the tumors derived from Ang-1 overexpressing cells led to several small tumors within the left lobe of the liver. This was likely due to diffusion through the sinusoidal spaces. In the control group, individual tumor masses coalesced to form large tumors whereas tumor growth was inhibited when Ang-1 cells were present. Therefore, when several tumors were present, the largest was used for evaluation. Tumor volumes from cell mixtures that contained either 50% or 10% Ang-1-transfected cells were significantly lower than tumor volumes in the 100% pcDNA-group (P<0.04 for both) (FIG. 13). Cell suspensions containing only Ang-1-transfected (100%) cells formed significantly smaller tumor volumes compared to controls (100% pcDNA) (P<0.04). Liver weights in the 100% Ang-1 group were again significantly lower compared to controls (100% pcDNA) (P<0.03).

### Example 15

### Effect of Recombinant Ang-1 on Non-Neoplastic Angiogenesis

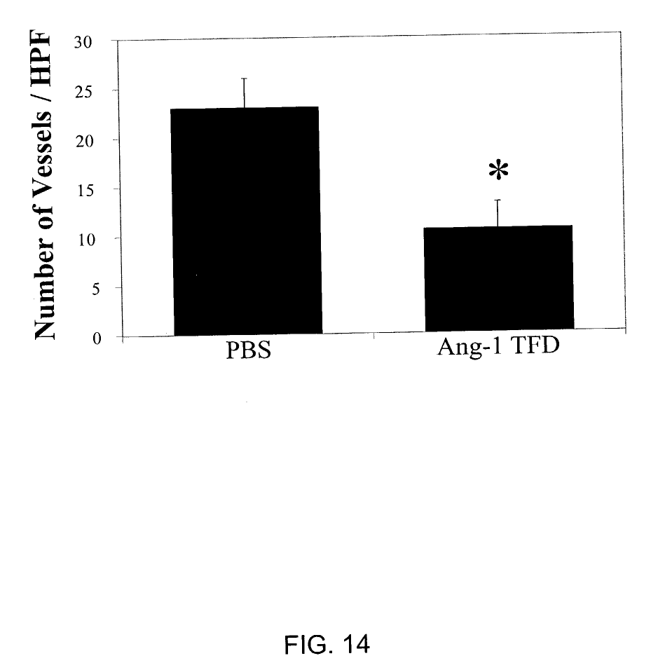

To demonstrate that the inhibition of hepatic tumor growth was mediated by inhibition of angiogenesis, the effects of recombinant human Ang-1 (Ang-1 TFD) on non-neoplastic angiogenesis in vivo were characterized. For this purpose, we used a Gelfoam in vivo angiogenesis assay was used in which agarose-Gelfoam sponges were soaked with either Ang-1 (1.0 μg Ang-1 TFD per μl of PBS) or PBS alone (control) and implanted subdermally in mice. Significantly fewer microvessels were present in the Ang-1-soaked Gelfoam plugs than in the PBS control Gelfoam plugs (P<0.02) (FIG. 14).

### Example 16

### Effect of Conditioned Media of Ang-1-Transfected Cells on Vascular Permeability

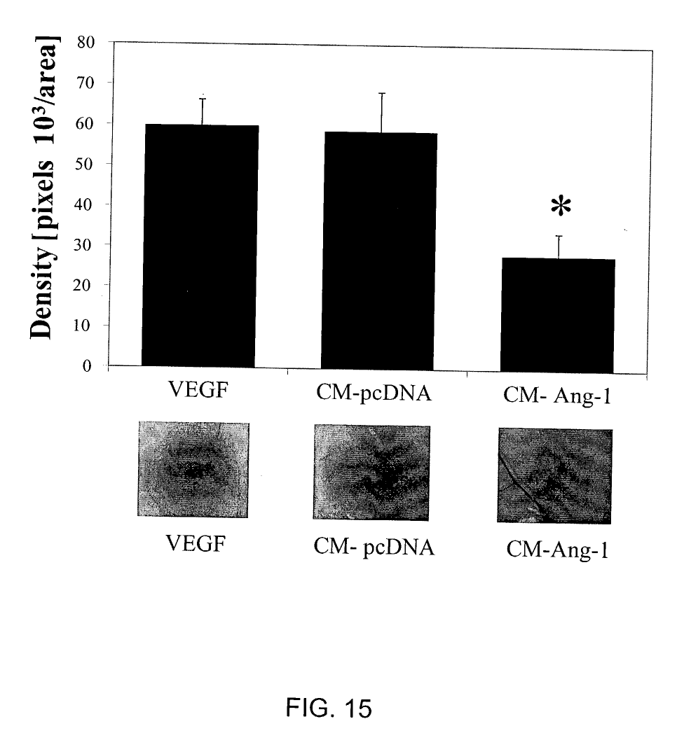

Finally, the effect of Ang-1 overexpression by human colon cancer cells on the permeability of resting vasculature was determined by using an intradermal Miles in vivo permeability assay, using conditioned media of transfected cell lines. Conditioned medium from Ang-1-transfected cells abrogated the pro-permeability effects mediated by tumor cell-derived growth factors. HT29 cells produce relatively high amounts of VEGF compared with other cell lines (Visconti et al., 2002). ELISA of conditioned media from both control and experimental cells contained similar levels of VEGF protein. Conditioned media from Ang-1-transfected cells led to a significant decrease in the overall area of dye leakage (8.0±0.9 mm² (mean±SEM)) as compared with conditioned media from pcDNA-transfected cells (29.3±3.7 mm2) (P<0.05). Densitometric analysis of digital images of all injection sites confirmed that Evans blue dye density at the CM-Ang-1 injection sites was significantly less than that at the CM-pcDNA injection sites (P<0.01) (FIG. 15).

### Example 17

### Effect of Endothelial Cell Co-Culture on Tie-2 Phosphorylation

In order verify that Ang-1 transfected HT29 cells produce functionally relevant amounts of Ang-1, ECs were co-cultured for 48 h with either Ang-1 or pcDNA transfected colon cancer cells. The effect on Tie-2 phosphorylation was assessed by immuoprecipitation and Western blot analysis. By densitometry, there was a ˜2-fold increase in Tie-2 phosphorylation in HUVECs exposed to Ang-1 transfected cells, compared to Tie-2 phosphorylation levels in HUVEC co-cultured with pcDNA HT29 cells.

### Example 18

### Significance of the Present Invention

The importance of the angiopoietins in embryonic angiogenesis has been clearly established (Maisonpierre et al., 1997; Sato et al., 1995; Dumont et al., 1994). However, their role in tumor angiogenesis remains to be elucidated. Angiopoietins have been implicated in vessel cooption and survival of primary and metastatic tumors (Holash et al., 1999). VEGF and angiopoietins seem to play complementary and coordinated roles in vascular development to support new tumors. Briefly, low-level constitutive expression of Ang-1 by normal tissue stabilizes existing blood vessels. Ang-2 overexpression by newly formed tumor blood vessels leads to vessel destabilization and relative hypoxia. This hypoxia drives the release of VEGF, which leads to robust angiogenesis.

The current Examples demonstrate the role of angiopoietins in colon cancer by showing the effects of Ang-1 and Ang-2 overexpression on tumor growth and angiogenesis in a xenograft model. The present invention demonstrates that Ang-2 overexpression was associated with marked increases in tumor growth rate, vessel count, and proliferation. These observations are consistent with the proposed hypothesis that Ang-2, in the presence of VEGF, induces the formation of new blood vessels (Peters, 1998). Ang-2 is constitutively expressed in colon cancer cells and in tumor endothelium. VEGF is also expressed by all colon cancer cell lines studied to date (Ellis et al., 1996; Warren et al., 1995). In adults, Ang-2 is expressed primarily at sites of vascular remodeling such as the ovaries, uterus, and placenta (Maisonpierrre et al., 1997), where it is thought to block the constitutive stabilizing action of Ang-1. The destabilizing effects of Ang-2 in the absence of VEGF have been suggested to lead to vessel regression. In another study, Tanaka et al. (Tanaka et al., 1999) injected the livers of nude mice with human HuH7 hepatocellular cancer cells that overexpressed Ang-2; all of these mice died from extensive intraperitoneal bleeding and the formation of large tumors. These findings support the present data that overexpression of Ang-2 leads to increased tumor growth.

The present invention also demonstrates that overexpression of Ang-1 in colon cancer xenografts led to the production of fewer tumor vessels, a finding that is consistent with the known stabilizing action of Ang-1. Ang-1 acts via the Tie-2 receptor and is thought to help maintain and stabilize mature vessels by promoting interactions between ECs and surrounding support cells (maisonpierrre et al., 1997; Suri et al., 1998; Asahara et al., 1998). Ang-1 also leads to increased Akt activation in ECs, thus enhancing survival signals (Papapetropoulos et al., 2000). Ang-1 is widely expressed in adult tissues (Tanaka et al., 1999), a reflection of its role in maintaining previously developed and mature blood vessels. Thus, the finding that the tumors from the mice injected with Ang-1-transfected cells had fewer tumor vessels than the other groups reflects the stabilizing action of Ang-1 on the endothelium. In a specific embodiment of the present invention an overproduction of Ang-1 inhibits or slows angiogenesis because of this stabilizing effect. Conversely, overproduction of Ang-2, from any cell type, in specific embodiments of the present invention, induce angiogenesis and subsequent tumor growth in an in vivo system. This was confirmed by the finding presented herein that the tumors consisting of Ang-2-transfected cells had not only more vessels but also higher tumor cell proliferation.

The present invention also demonstrates the expression patterns of Ang-1 and Ang-2 in colon cancer by analyzing their expression in cultured colon cancer cell lines, in primary human colon cancers and nearby normal colon mucosa, and in colorectal liver metastases. Primary and metastatic colon cancers frequently expressed Ang-2 and infrequently expressed Ang-1. Normal colon mucosa and liver tissue expressed both Ang-1 and Ang-2. The localization studies (double-staining for CD31 and Ang-1 or Ang-2) showed that both angiopoietins were present in the endothelial and periendothelial cells. In an attempt to clarify the exact origin of angiopoietin expression, double-staining with antibodies were performed to Ang-1 or Ang-2 and cytokeratin-22 or smooth muscle actin. It was found that both Ang-1 and Ang-2 were expressed in endothelial cells, pericytes, and colon mucosa, but Ang-2 was also present in colon cancer cells, whereas Ang-1 was not.

The finding that Ang-2 was expressed by colonic epithelium is in contrast to that of Tanaka et al. (1999) who examined 23 samples of human hepatocellular carcinoma (HCC) for the expression of Ang-1 and Ang-2 by immunohistochemistry. In that study, Ang-1 was found to be equally expressed in HCC and in adjacent normal liver tissue, but Ang-2 was highly expressed only in tumor tissue. The findings also contrast with those of Zagzag et al. (1999) and Stratmann et al. (1998) who used in situ hybridization to examine human astrocytoma and glioblastoma tumors, respectively, for the expression of angiopoietins. In both studies, Ang-1 mRNA was found in tumor cells and Ang-2 mRNA in tumor blood vessel endothelial cells; in neither study was Ang-1 or Ang-2 found in normal brain tissue.

Given the differences between the results presented herein and those of the few other published reports, several internal controls were used to validate the findings. First, the primary antibody was tested by using it to stain tumor cells that had been transfected with either Ang-1 or Ang-2 cDNA constructs. The increased expression of Ang-1 and Ang-2 protein in the transfected cell lines was confirmed. Second, double-staining was utilized to confirm the cell type from which the proteins originated; smooth muscle actin to identify pericytes and vascular smooth muscle cells and cytokeratin-22 to identify cells of epithelial origin. Finally, Ang-1 and Ang-2 were examined in tumor cell lines by RT-PCR and the infrequent expression of Ang-1 was verified in those cell lines. Although Ang-1 was found to be more frequently expressed in tumor cell lines by RT-PCR than by immunohistochemistry, this result likely reflects the greater sensitivity of RT-PCR. Although immunohistochemistry may not always be the best method for detecting the cellular source of protein, the studies presented herein evaluating the angiopoietins in cell lines, as well as human specimens by several techniques, further demonstrates that non-neoplastic and neoplastic epithelium are indeed an important source of these proteins. Furthermore, the absence of the receptor for these proteins on epithelial cells adds credence to the observations that these proteins are expressed by colonic epithelial cells, and not detected on epithelial cells by immunohistochemistry after binding to a receptor.

The role of angiopoietins in colon cancer angiogenesis has not been previously described. It is shown herein that an imbalance exists in colon cancer that seems to favor Ang-2 activity over that of Ang-1. Others have shown that Ang-1 works in conjunction with VEGF in angiogenesis and that Ang-1 is involved in vessel stabilization through increasing the association of endothelial cells with surrounding structural cells such as pericytes. (Suri et al., 1996) Ang-2, in specific embodiments, disrupts the stabilizing effect of Ang-1. The pattern of Ang-2 expression that was found suggests that Ang-2 plays an early role in vessel formation (Maisonpierre et al., 1997) and, with VEGF, induces a mitogenic response. In specific embodiments of the present invention, that response could take place in two ways. First, Ang-2 leads to endothelial cell destabilization, which can lead to hypoxia and upregulation of VEGF. (Holash et al., 1999). Second, disruption of the interaction between endothelial cells and pericytes could also decrease the stabilizing effect of pericytes, thereby allowing endothelial cells to proliferate. These embodiments are consistent with the finding of an imbalance favoring Ang-2 over Ang-1. Tumors may overexpress Ang-2 to induce new blood vessel formation; this formation, however, depends on the presence of VEGF when Ang-2 is overexpressed. Previous studies (Takahashi et al., 1995; Takahashi et al., 1997) have shown that VEGF expression is high in biologically aggressive and metastatic colon cancers. It was shown herein that colon cancers produce both VEGF and Ang-2; this co-production likely initiates tumor blood vessel formation in a synergistic fashion.

Thus, in colon cancer an imbalance exists such that the expression of Ang-2 exceeds that of Ang-1. This imbalance induces blood vessel destabilization and, in some embodiments in the presence of VEGF, increases angiogenesis and tumor growth of colorectal cancers. Further, angiopoietins seem to be produced not only by tumor cells but also by periendothelial cells, indicating that tumor angiogenesis involves dynamic interactions between host cells (endothelial cells and pericytes) and tumor cells.

Thus, as demonstrated herein, overrexpression of angiopoietin-1 reduces tumor growth and angiogenesis of colon cancer hepatic metastases and prevents ascites formation in a peritoneal carcinomatosis model of colorectal cancer by reducing vascular permeability. This indicates that angiopoietin-1 is useful for treatment of cancer, particularly of colon cancer and liver cancer. In a specific embodiment, the present invention is useful for peritoneal carcinomatosis. Examples of peritoneal surface malignancies include appendix cancer and pseudomyxoma peritonei, colon cancer with peritoneal carcinomatosis, gastric cancer with peritoneal carcinomatosis, abdominopelvic sarcoma with sarcomatosis, and primary peritoneal surface malignancy including peritoneal mesothelioma, papillary serous cancer, and primary peritoneal adenocarcinoma.

Furthermore, the present inventors also report three in vivo effects of Ang-1 in angiogenesis and regulation of vascular permeability: (a) overexpression of Ang-1 by HT29 human colon cancer cells inhibited tumor angiogenesis and the growth of hepatically implanted tumor cells in mice; (b) Ang-1 inhibited non-neoplastic angiogenesis in an in vivo angiogenesis assay; and (c) Ang-1 abrogated the pro-permeability effects of tumor cell-derived growth factors. The anti-angiogenic effect of Ang-1, in specific embodiments, is mediated by the recruitment of periendothelial supporting cells (pericytes), leading to an overall vessel stabilization.

The functional complexity of angiopoietins in the regulation of angiogenesis and in their effects on tumor growth is mirrored by conflicting reports on the in vivo effects of Ang-1 and Tie-2 activation (Ahmad et al., 2001; Suri et al., 1998; Lin et al., 1998; Chae et al., 2000; Hayes et al., 2000; Hansbury et al., 2001; Hangai et al., 2001; Shim et al., 2001). Initially, Holash et al. elegantly demonstrated the importance of coordinated induction of angiopoietins and VEGF in tumor angiogenesis (Holash et al., 1999). Several studies have suggested that Ang-1 may be, in general, pro-angiogenic (Peters, 1998; Ray et al., 2000; Thurston et al., 1999; Suri et al., 1998; Shim et al., 2001). Increased neovascularization by Ang-1 was demonstrated in transgenic mouse models, where Ang-1 overexpression by keratinocytes—in combination with endogenous VEGF expression-led to increased dermal vascularization in mice, suggesting that VEGF and Ang-1 play coordinated and complementary roles. Thus, it was suggested that Ang-1 be used in combination with VEGF for promoting therapeutic angiogenesis (Thurston et al., 2000; Suri et al., 1998). The importance of cooperation of Ang-1 and VEGF for promotion of angiogenesis has been demonstrated in several malignant and non-malignant models of angiogenesis (Peters, 1998; Ray et al., 2000). However, until recently, only a few reports were available on the role of the angiopoietins in tumor angiogenesis. In contrast to our previous report on the effects of imbalances in Ang-1 and -2 expression in colon cancer cells, where Ang-1 overexpression inhibited angiogenesis and growth of xenografted tumors (Ahmad et al., 2001), Shim et al. demonstrated that antisense Ang-1 mRNA expression by HeLa cervical adenocarcinoma cells inhibited angiogenesis and growth of xenografted tumors in immunodeficient mice (Shim et al., 2001). A different approach to Ang-1 inhibition was used by Lin et al., who demonstrated that a soluble Tie-2 receptor could decrease angiogenesis and tumor growth of murine melanoma and mammary tumors when delivered by an adenoviral vector (Lin et al., 1998).

Recently, several studies suggested that overexpression or administration of Ang-1 may inhibit both neoplastic and non-neoplastic angiogenesis (Hawighorst et al., 2002; Hayes et al., 2000; Tian et al., 2002; Joussen et al., 2002). Joussen et al. investigated the effects of intravitreal Ang-1 application on retinal vascularization in diabetic rats. In that study, Ang-1 decreased retinal neovascularization and normalized VEGF levels. Similar results were found when Ang-1 was delivered systemically by an adenoviral vector (Joussen et al., 2002). A blunted pro-angiogenic effect of VEGF by Ang-1 was also described by Visconti et al. in a transgenic mouse model of cardiac-specific expression or co-expression of Ang-1, Ang-2, or VEGF (Visconti et al., 2002). The present inventors previously demonstrated that imbalances in angiopoietin expression may regulate growth and angiogenesis of human colon cancer. Ang-1 overexpression significantly inhibited tumor angiogenesis in that xenograft model (Ahmad et al., 2001). The present Examples showed that overexpression of Ang-1 significantly reduced tumor growth (79%) and neovascularization (25%), this time in a model of colorectal cancer growing at the preferred site for metastases (i.e. liver). Additionally, by using various mixtures of cell suspensions of Ang-1- and pcDNA-transfected cells, the present inventors were able to demonstrate for the first time that Ang-1 secreted by tumor cells impacts the growth of tumor cells that do not express Ang-1. In this experiment, a mixture of 10% Ang-1-transfected cells with 90% of pcDNA-transfected cells was sufficient to significantly reduce tumor growth in this group compared to the 100% pcDNA group. This suggests, that adding Ang-1 to the tumor microenvironment may significantly inhibit the angiogenic process, resulting in an overall inhibition of tumor growth. The anti-angiogenic effect of Ang-1 observed in our study may be mediated in part by increased periendothelial support by pericytes (high pericyte coverage in Ang-1 tumors), resulting in an overall vessel stabilization and thereby inhibition of initiation of tumor angiogenesis. As HT29 cells do not express Tie-2, the observed effects of Ang-1 expression on tumor growth result from effects on ECs and periendothelial cells rather than from effects on tumor cells themselves. In apparent contrast to these findings are those from Stratman et al., who investigated the effects of Tie-2 inhibition in breast cancer cell lines that express various levels of Tie-2 by stable transfection with a dominant-negative form of the Tie-2 receptor. Their results showed a 15% growth inhibition after transfection into a Tie-2 negative cell line, and a 57% inhibition of a Tie-2 positive cell line, respectively. However, these observed effects are difficult to explain with respect to angiogenesis (Stratmann et al., 2001).

Ang-1 also inhibited non-neoplastic neovascularization, as demonstrated by our in vivo angiogenesis assay with a novel recombinant Ang-1 (Ang-1 TFD) (Davis et al., 2003). The finding that the Gelfoam plugs were negative for α-SMA expression suggests that Ang-1 has a direct inhibitory effect on ECs in vivo.

The findings provided herein are supported by the results of a recent study in which Ang-1 overexpression by MCF-7 breast cancer cells resulted in stabilization of blood vessels associated with the tumor edge (Tian et al., 2002). In that study, tumor cell proliferation decreased significantly in the presence of Ang-1 and prevented vessel dilation and dissociation of smooth muscle cells from existing vessels, which resulted in reductions in xenografted tumor growth. On the basis of results from their Matrigel in vivo assay in which Ang-1 increased mesenchymal cell infiltration, Tian et al. concluded that vascular stabilization by Ang-1 accounts for the inhibition of tumor growth. They also demonstrated that Tie-2 was expressed on smooth muscle cells in culture (Tian et al., 2002). In a previous study, Hayes et al. also demonstrated that Ang-1 overexpression in MCF-7 human breast cancer cells caused a significant retardation in tumor growth despite the high co-expression of a potent angiogenic growth factor (fibroblast growth factor-1) (Hayes et al., 2000). The same growth-inhibitory effect (70% reduction) by Ang-1 was observed by Hawighorst et al. in stable transfected human squamous cell carcinoma cells. Those authors did not detect changes in vessel density, but confirmed a significant increase in pericyte-covered vessels in Ang-1-transfected tumors (Hawighorst et al., 2002). Results, from our study expand these findings showing that Ang-1 in the tumor microenvironment may also recruit pericytes into hepatic metastasis. Sundberg et al. recently described that pericytes express Ang-1 at later stages of the angiogenic process, leading to further vessel stabilization and maturation of the tumor neovascular network (Sundberg et al., 2002). Taken together, these studies indicate that continuous Tie-2 activation on ECs leads to increased vessel stabilization, in specific embodiments, thereby making the vasculature less susceptible to pro-angiogenic factors such as VEGF.

Vessel stabilization by Ang-1 is associated with decreased vascular permeability. In the in vivo permeability assay provided herein, Ang-1 levels in conditioned medium from Ang-1-transfected cells abrogated the increase of plasma leakage (dye leakage) caused by tumor cell-derived growth factors. Similar results were obtained with conditioned medium from transfected KM12L4 cells (high constitutive VEGF expression (Ellis et al., 1996)), suggesting that Ang-1 is an important mediator of vascular stabilization and permeability and may override VEGF-mediated vessel leakage. This phenomenon has been described by other groups who have investigated the effects of Ang-1 on vascular permeability and vessel stabilization (Thurston et al, 1999; Thurston et al., 2000). Thurston et al. described anti-permeability properties of Ang-1 in two different studies, one evaluating the effect of VEGF on plasma leakage of adult vasculature and another with a transgenic mouse model in which both Ang-1 and VEGF were overexpressed (Thurston et al, 1999; Thurston et al., 2000). In the mouse study, co-expression of Ang-1 and VEGF resulted in the formation of leakage-resistant vessels (Thurston et al., 1999). The authors also showed that acute administration of Ang-1 protected adult vasculature from leakage mediated by VEGF and inflammatory cytokines (Thurston et al., 2000). The molecular mechanism of this regulatory effect was recently described by Gamble et al., who showed that administration of recombinant Ang-1 supported the localization of a cell adhesion molecule (PECAM-1) into junctions between endothelial cells, thereby strengthening these junctions (Gamble et al., 2000).

Thus, the present inventors indicate that Ang-1 expression or administration may negatively regulate angiogenesis and decrease vascular permeability by stabilizing ECs and increasing periendothelial support. Thus, Ang-1 is an important mediator of neoplastic and non-neoplastic angiogenesis; however, its precise role in this process remains to be elucidated. Sequential expression of Ang-1, Ang-2, and VEGF has been shown to be crucial for successful angiogenesis (Asahara et al., 1998;). Therefore, any interruption or disturbance in this balanced expression will probably affect the angiogenic process significantly. Such a disturbance could occur at the level of continuous Tie-2 activation (by Ang-1) or by Tie-2 interruption (soluble Tie-2, Tie-2 receptor antagonists). In preferred embodiments of the present invention, Ang-1 is useful as an anti-angiogenic and/or anti-permeability agent in the treatment of cancer, and particularly of metastatic colorectal cancer.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.

### Patents

U.S. Pat. No. 4,554,101

U.S. Pat. No. 5,521,073

U.S. Pat. No. 5,650,490

U.S. Pat. No. 5,643,755

U.S. Pat. No. 5,879,672

U.S. Pat. No. 6,156,497

U.S. Pat. No. 6,228,646

### Publications

Ahmad, S. A., Liu, W., Jung, Y. D., Fan, F., Reinmuth, N., Bucana, C. D., and Ellis, L. M. Differential expression of angiopoietin-1 and angiopoietin-2 in colon carcinoma. A possible mechanism for the initiation of angiogenesis. Cancer, 92: 1138-1143, 2001.

Ahmad, S. A., Liu, W., Jung, Y. D., Fan, F., Wilson, M., Reinmuth, N., Shaheen, R. M., Bucana, C. D., and Ellis, L. M. The effects of angiopoietin-1 and -2 on tumor growth and angiogenesis in human colon cancer. Cancer Res, 61: 1255-1259, 2001.

Audero, E., Cascone, I., Zanon, I., Previtali, S. C., Piva, R., Schiffer, D., and Bussolino, F. Expression of angiopoietin-1 in human glioblastomas regulates tumor-induced angiogenesis: in vivo and in vitro studies. Arterioscler Thromb Vasc Biol, 21: 536-541, 2001.

Asahara, T., Chen, D., Takahashi, T., Fujikawa, K., Kearney, M., Magner, M., Yancopoulos, G. D., and Isner, J. M. Tie2 receptor ligands, angiopoietin-1 and angiopoietin-2 modulate VEGF-induced postnatal neovascularization. Circ. Res., 83: 233-240, 1998.

Bruns, C. J., Liu, W., Davis, D. W., Shaheen, R. M., McConkey, D. J., Wilson, M. R., Bucana, C. D., Hicklin, D. J., and Ellis, L. M. Vascular endothelial growth factor is an in vivo survival factor for tumor endothelium in a murine model of colorectal carcinoma liver metastases. Cancer, 89: 488-499, 2000.

Chae, J. K., Kim, I., Lim, S. T., Chung, M. J., Kim, W. H., Kim, H. G., Ko, J. K., and Koh, G. Y. Coadministration of angiopoietin-1 and vascular endothelial growth factor enhances collateral vascularization. Arterioscler Thromb Vase Biol, 20: 2573-2578, 2000.

Davis, S., Papadopoulos, N., Aldrich, T. H., Maisonpierre, P. C., Huang, T., Kovac, L., Xu, A., Leidich, R., Radziejewska, E., Rafique, A., Goldberg, J., Jain, V., Bailey, K., Karow, M., Fandl, J., Samuelsson, S. J., Ioffe, E., Rudge, J. S., Daly, T. J., Radziejewski, C., and Yancopoulos, G. D. Angiopoietins have distinct modular domains essential for receptor binding, dimerization and superclustering. Nat Struct Biol, 10: 38-44, 2003.

Dong, Z., Radinsky, R., Fan, D., Tsan, R., Bucana, C. D., Wilmanns, C., and Fidler, I. J. Organ-specific modulation of steady-state mdr-1 gene expression and drug resistance in murine colon cancer cells. J. Natl. Cancer Inst., 86: 913-920, 1994.

Dumont, D. J., Gradwohl, G., Fong, G. H., Puri, M. C., Gertsenstein, M., Auerbach, A., and Breitman, M. L. Dominant-negative and targeted null mutations in the endothelial receptor tyrosine kinase, tek, reveal a critical role in vasculogenesis of the embryo. Genes Dev., 1909, 8:1897, 1994.

Eberhard, A., Kahlert, S., Goede, V., Hemmerlein, B., Plate, K. H., and Augustin, H. G. Heterogeneity of angiogenesis and blood vessel maturation in human tumors: implications for antiangiogenic tumor therapies. Cancer Res, 60: 1388-1393, 2000.

Ellis, L. M., Liu, W., and Wilson, M. Down-regulation of vascular endothelial growth factor in human colon carcinoma cell lines by antisense transfection decreases endothelial cell proliferation. Surgery, 120: 871-878, 1996.

Fidler, I. J. Angiogenesis and cancer metastasis. Cancer Journal From Scientific American, 6 Suppl 2: S134-141, 2000.

Folkman, J. The role of angiogenesis in tumor growth. Semin Cancer Biol, 3: 65-71, 1992.

Folkman, J. Angiogenesis in cancer, vascular, rhuematoid and other disease. Nature Med, 1: 27-31, 1995.

Ellis, L. M., Liu., W., and Wilson, M. Down-regulation of vascular endothelial growth factor in human colon carcinoma cell lines by antisense transfection decreases endothelial cell proliferation. Surgery, 120: 871-878, 1996.

Gamble, J. R., Drew, J., Trezise, L., Underwood, A., Parsons, M., Kasminkas, L., Rudge, J., Yancopoulos, G., and Vadas, M. A. Angiopoietin-1 is an antipermeability and anti-inflammatory agent in vitro and targets cell junctions. Circ Res, 87: 603-607, 2000.

Hangai, M., Moon, Y. S., Kitaya, N., Chan, C. K., Wu, D. Y., Peters, K. G., Ryan, S. J., and Hinton, D. R. Systemically expressed soluble Tie2 inhibits intraocular neovascularization. Hum Gene Ther, 12: 1311-1321, 2001.

Hansbury, M. J., Nicosia, R. F., Zhu, W. H., Holmes, S. J., and Winkler, J. D. Production and characterization of a Tie2 agonist monoclonal antibody. Angiogenesis, 4: 29-36, 2001.

Hardy et al. (Sep. 25-29, 1996) “A Gutless Adenovirus Vector For Gene Therapy,” CSHL Gene Therapy Meeting, Abstract, p. 156.

Hawighorst, T., Skobe, M., Streit, M., Hong, Y. K., Velasco, P., Brown, L. F., Riccardi, L., Lange-Asschenfeldt, B., and Detmar, M. Activation of the tie2 receptor by angiopoietin-1 enhances tumor vessel maturation and impairs squamous cell carcinoma growth. Am J Pathol, 160: 1381-1392, 2002.

Hayes, A. J., Huang, W. Q., Yu, J., Maisonpierre, P. C., Liu, A., Kern, F. G., Lippman, M. E., McLeskey, S. W., and Li, L. Y. Expression and function of angiopoietin-1 in breast cancer. Br J Cancer, 83: 1154-1160, 2000.

Hirschi KK, Rohovsky SA, Beck LH, Smith SR, D'Amore PA: Endothelial cells modulate the proliferation of mural cell precursors via platelet-derived growth factor-BB and heterotypic cell contact. Circ Res 1999; 84:298-305.

Holash, J., Maisonpierre, P. C., Compton, D., Boland, P., Alexander, C. R., Zagzag, D., Yancopoulos, G. D., and Wiegand, S. J. Vessel cooption, regression, and growth in tumors mediated by agiopoietins and VEGF. Science, 284: 1994-1998, 1999.

Joussen, A. M., Poulaki, V., Tsujikawa, A., Qin, W., Qaum, T., Xu, Q., Moromizato, Y., Bursell, S. E., Wiegand, S. J., Rudge, J., loffe, E., Yancopoulos, G. D., and Adamis, A. P. Suppression of diabetic retinopathy with angiopoietin-1. Am J Pathol, 160: 1683-1693, 2002.

Kwak, H. J., So, J. N., Lee, S. J., Kim, I., and Koh, G. Y. Angiopoietin-1 is an apoptosis survival factor for endothelial cells. FEBS Lett 448: 249-253, 1999.

Lin, P., Buxton, J. A., Acheson, A., Radziejewski, C., Maisonpierre, P. C., Yancopoulos, G. D., Channon, K. M., Hale, L. P., Dewhirst, M. W., George, S. E., and Peters, K. G. Antiangiogenic gene therapy targeting the endothelium-specific receptor tyrosine kinase Tie2. Proc Natl Acad Sci USA, 95: 8829-8834, 1998.

Liu, W., Davis, D. W., Ramirez, K., McConkey, D. J., and Ellis, L. M. Endothelial cell apoptosis is inhibited by a soluble factor secreted by human colon cancer cells. Int J Cancer, 92: 26-30, 2001.

Maisonpierre, P. C., Suri, C., Jones, P. F., Barunkova, S., Wiegand, S. J., Radziejewski, C., Compton, D., McClain, J., Aldrich, T. H., Papadoupoulos, N., Daly, T. J., Davis, S., Sato, T. N., and Yancopoulos, G. D. Angiopoietin-2, a natural antagonist for Tie 2 that disrupts in vivo angiogenesis. Science, 277: 55-60, 1997.

McCarty, M. F., Baker, C. H., Bucana, C. D., and Fidler, I. J. Quantitative and qualitative in vivo angiogenesis assay. Int J Oncol, 21: 5-10, 2002.

Mitsutake, N., Namba, H., Takahara, K., Ishigaki, K., Ishigaki, J., Ayabe, H., and Yamashita, S. Tie-2 and angiopoietin-1 expression in human thyroid tumors. Thyroid, 12: 95-99, 2002.

Papapetropoulos, A., Fulton, D., Mahboubi, K., Kalb, R. G., O'Connor, D. S., Li, F., Altieri, D. C., and Sessa, W. C. Angiopoietin-1 inhibits endothelial cell apoptosis via the Akt/Survivin pathway. J. Biol. Chem., 275: 9102-9105, 2000.

Papapetropoulos, A., Guillermo, G. C., Dengler, T. J., Maisonpierre, P. C., Yancopoulos, G. D., and Sessa, W. C. Direct actions of angiopoietin-1 on human endothelium: evidence for network stabilization, cell survival, and interaction with other angiogenic growth factors. Lab. Invest., 79: 213-223, 1999.

Peters, K. G. Vascular endothelial growth factor and the angiopoietins: working together to build a better blood vessel. Circ. Res., 83: 342-343, 1998.

Pomyje, J., Zivny, J. H., Stopka, T., Simak, J., Vankova, H., and Necas, E. Angiopoietin-1, angiopoietin-2 and Tie-2 in tumour and non-tumour tissues during growth of experimental melanoma. Melanoma Res, 11: 639-643, 2001.

Ray, P. S., Estrada-Hemandez, T., Sasaki, H., Zhu, L., and Maulik, N. Early effects of hypoxia/reoxygenation on VEGF, ang-1, ang-2 and their receptors in the rat myocardium: implications for myocardial angiogenesis. Mol Cell Biochem, 213: 145-153, 2000.

Reinmuth, N., Liu, W., Fan, F., Jung, Y. D., Ahmad, S. A., Stoeltzing, O., Bucana, C. D., Radinsky, R., and Ellis, L. M. Blockade of insulin-like growth factor I receptor function inhibits growth and angiogenesis of colon cancer. Clin Cancer Res, 8: 3259-3269, 2002.

Sato, T. N., Tozawa, Y., Deutsch, U., Wolburg-Buchholz, K., Fujiwara, Y., Gendron-Maguire, M., Gridley, T., Wolburg, H., Risau, W., and Quin, Y. Distinct roles of the receptor tyrosine kinases Tie-1 and Tie-2 in blood vessel formation. Nature 376: 70-74, 1995.

Shaheen, R. M., Ahmad, S. A., Liu, W., Reinmuth, N., Jung, Y. D., Tseng, W. W., Drazan, K. E., Bucana, C. D., Hicklin, D. J., and Ellis, L. M. Inhibited growth of colon cancer carcinomatosis by antibodies to vascular endothelial and epidermal growth factor receptors. Br J Cancer, 85: 584-589, 2001.

Shim, W. S., Teh, M., Mack, P. O., and Ge, R. Inhibition of angiopoietin-1 expression in tumor cells by an antisense RNA approach inhibited xenograft tumor growth in immunodeficient mice. Int J Cancer, 94: 6-15, 2001.

Stratman, A., Risau, W., and Plate, K. H. Cell type-specific expression of angiopoietin-1 and angiopoietin-2 suggests a role in glioblastoma angiogenesis. Am. J. Pathol., 153: 1459-1466, 1998.

Stratmann, A., Acker, T., Burger, A. M., Amann, K., Risau, W., and Plate, K. H. Differential inhibition of tumor angiogenesis by tie2 and vascular endothelial growth factor receptor-2 dominant-negative receptor mutants. Int J Cancer, 91: 273-282, 2001.

Sundberg, C., Kowanetz, M., Brown, L. F., Detmar, M., and Dvorak, H. F. Stable expression of angiopoietin-1 and other markers by cultured pericytes: phenotypic similarities to a subpopulation of cells in maturing vessels during later stages of angiogenesis in vivo. Lab Invest, 82: 387-401, 2002.

Suri, C., Jones, P. F., Patan, S., Bartunkova, S., Maisonpierre, P. C., Davis, S., Sato, T. N., and Yancopoulos, G. D. Requisite role of angiopoietin-1, a ligand for the tie2 receptor, during embryonic angiogenesis. Cell, 87: 1171-1180, 1996.

Suri, C., McClain, J., Thurston, G., McDonald, D. M., Zhou, H., Olmixon, E. H., Sato, T. N., and Yancopoulos, G. D. Angi

Suri, C., McClain, J., Thurston, G., McDonald, D. M., Zhou, H., Oldmixon, E. H., Sato, T. N., and Yancopoulos, G. D. Increased vascularization in mice overexpressing angiopoietin-1. Science, 282: 468-471, 1998.

Takahashi, Y., Bucana, C. D., Cleary, K. R., and Ellis, L. M. p53, vessel count, and vascular endothelial growth factor expression in human colon cancer. Int J Cancer, 79: 34-38, 1998.

Takahashi Y, Kitadai Y, Bucana CD, Cleary KR, Ellis LM: Expression of vascular endothelial growth factor and its receptor, KDR, correlates with vascularity, metastasis, and proliferation of human colon cancer. Canc Res 1995; 55:3964-3968.

Takahashi Y, Tucker SL, Kitadai Y, Koura AN, Bucana CD, Cleary KR, et al: Vessel counts and VEGF expression as prognostic factors in node-negative colon cancer. Arch Surg 1997; 132:541-546.

Tanaka, S., Mori, M., Sakamoto, Y., Makuuchi, M. N., Sugimachi, K., and Wands, J. R. Biologic significance of angiopoietin—

Thurston, G., Suri, C., Smith, K., McClain, J., Sato, T. N., Yancopoulos, G. D., and McDonald, D. M. Leakage-resistant blood vessels in mice transgenically overexpressing angiopoietin-1. Science, 286: 2511-2514, 1999.

Thurston, G., Rudge, J. S., loffe, E., Zhou, H., Ross, L., Croll, S. D., Glazer, N., Holash, J., McDonald, D. M., and Yancopoulos, G. D. Angiopoietin-1 protects the adult vasculature against plasma leakage. Nat Med, 6: 460-463, 2000.

Tian, S., Hayes, A. J., Metheny-Barlow, L. J., and Li, L. Y. Stabilization of breast cancer xenograft tumour neovasculature by angiopoietin-1. Br J Cancer, 86: 645-651, 2002.

Visconti, R. P., Richardson, C. D., and Sato, T. N. Orchestration of angiogenesis and arterioyenous contribution by angiopoietins and vascular endothelial growth factor (VEGF). Proc Natl Acad Sci USA, 2002.

Warren, R. S., Yuan, H., Matli, M. R., Gillett, N. A., and Ferrara, N. Regulation by vascular endothelial growth factor of human colon cancer tumorigenesis in a mouse model of experimental liver metastases. J. Clin. Invest., 95: 1789-1797, 1995.

Witzenbichler, B., Maisonpierre, P. C., Jones, P., Yancopoulos, G. D., and Isner, J. M. Chemotactic properties of angiopoitin-1 and -2, ligands for the endothelial-specific receptor tyrosine kinase Tie2. J. Biol. Chem., 273: 18514-18521, 1998.

Wong, A. L., Haroon, Z. A., Werner, S., Dewhirst, M. W., Greenberg, C. S., and Peters, K. G. Tie 2 expression and phosphorylation in angiogenic and quiescent adult tissues. Circ. Res., 81: 567-574, 1997.

Zagzag, D., Hooper, A., Friedlander, D. R., Chan, W., Holash, J., Wiegand, S. J., Yancopoulos, G. D., and Grumet, M. In situ expression of angiopoietins in astrocytomas identifies angiopoietin-2 as an early marker of tumor angiogenesis. Exp. Neurol., 159: 391-400, 1999.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, manufacture, composition of matter, means, methods and steps described in the specification. As one of ordinary skill in the art will readily appreciate from the disclosure of the present invention, processes, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

We claim:

1. A method of stabilizing the endothelium or reducing endothelial cell proliferation associated with a tumor, the method comprising administering to a patient having a tumor an amount of angiopoietin-1 polypeptide effective to stabilize the endothelium or reduce endothelial cell proliferation.

2. The method of claim 1, wherein the angiopoietin-1 polypeptide is introduced to the tumor through the introduction of an angiopoietin-1-encoding polynucleotide.

3. The method of claim 1, wherein the tumor is a colon tumor.

4. The method of claim 1, wherein the tumor is a colorectal tumor.

5. The method of claim 1, wherein the tumor is a liver tumor.

6. The method of claim 1, wherein the tumor is a peritoneal carcinomatosis.

7. The method of claim 6, wherein the peritoneal carcinomatosis is appendix cancer, pseudomyxoma peritonei, colon cancer with peritoneal carcinomatosis, gastric cancer with peritoneal carcinomatosis, abdominopelvic sarcoma with sarcomatosis, or a primary peritoneal surface malignancy.

8. The method of claim 7, wherein the primary peritoneal surface malignancy is peritoneal mesothelioma, papillary serous cancer, or primary peritoneal adenocarcinoma.

9. The method of claim 1, wherein the tumor is in a patient.

10. The method of claim 9, wherein the angiopoietin-1 polypeptide is contacted with the tumor by injection into the tumor.

11. The method of claim 9, wherein the contacting step is further defined as injecting a polynucleotide encoding the angiopoietin-1 polypeptide into the patient.

12. The method of claim 11, wherein the injection is orthotopic, intradermal, subcutaneous, intramuscular, intraperitoneal, or intravenous.

13. The method of claim 11, wherein the injection is regional to the tumor.

14. The method of claim 9, further comprising treating the tumor with a second agent, wherein the second agent is a therapeutic polypeptide, polynucleotide encoding a therapeutic polypeptide, chemotherapeutic agent, or radiotherapeutic agent.

15. The method of claim 1, wherein the angiopoietin-1 polypeptide is administered by injection.

16. The method of claim 2, wherein said polynucleotide is a deoxyribonucleic acid molecule that encodes an angiopoietin-1 polypeptide.

17. The method of claim 2, wherein said angiopoietin-1-encoding polynucleotide further comprises control sequences operatively linked to said angiopoietin-1 encoding polynucleotide.

18. The method of claim 2, wherein said angiopoietin-1-encoding polynucleotide is located on a vector.

19. The method of claim 18, wherein said polynucleotide is operably linked to a promoter.

20. The method of claim 19, wherein said promoter is selected from the group consisting of CMV IE, SV40 IE, RSV LTR, or β-actin.

21. The method of claim 18, wherein said vector comprises a plasmid vector.

22. The method of claim 18, wherein said vector comprises a viral vector.

23. The method of claim 22, wherein said viral vector is selected from the group consisting of retrovirus, adenovirus, herpesvirus, vaccinia virus, and adeno-associated virus.

24. The method of claim 22, wherein the viral vector is an adenoviral vector.

25. A method of inhibiting angiogenesis related to cancer in an individual, comprising the steps of contacting a cell affected by the cancer with an angiopoietin-1 polypeptide in an amount effective to inhibit said angiogenesis.

26. The method of claim 25, wherein said angiopoietin-1 polypeptide is introduced into a cancer cell by the direct introduction of said angiopoietin-1 polypeptide.

27. The method of claim 25, wherein said angiopoietin-1 polypeptide is introduced into the cell through the introduction of an angiopoietin-1-encoding polynucleotide.

28. The method of claim 25, wherein the cancer is colon cancer.

29. The method of claim 25, wherein the cancer is colon cancer, liver cancer, or colorectal cancer.

30. The method of claim 25, wherein the cancer is a peritoneal adenocarcinoma.

31. The method of claim 25, further comprising treating the cell with a second agent, wherein the second agent is a therapeutic polypeptide, polynucleotide encoding a therapeutic polypeptide, chemotherapeutic agent, or radiotherapeutic agent.

32. The method of claim 25, wherein the angiopoietin-1 polypeptide is administered by injection.

33. The method of claim 27, wherein the angiopoietin-1 polynucleotide is administered to the individual by injection.

34. The method of claim 33, wherein the injection is orthotopic, intradermal, subcutaneous, intramuscular, intraperitoneal, or intravenous.

35. The method of claim 27, wherein the polynucleotide is administered with a liposome.

36. The method of claim 27, wherein said polynucleotide is a deoxyribonucleic acid molecule that encodes an angiopoietin-1 polypeptide.

37. The method of claim 36, wherein said angiopoietin-1-encoding polynucleotide further comprises control sequences operatively linked to said angiopoietin-1 encoding polynucleotide.

38. The method of claim 37, wherein said angiopoietin-1-encoding polynucleotide is located on a vector.

39. The method of claim 38, wherein said polynucleotide is operably linked to a promoter.

40. The method of claim 39, wherein said promoter is selected from the group consisting of CMV TE, SV40 IE, RSV LTR, or β-actin.

41. The method of claim 38, wherein said vector comprises a plasmid vector.

42. The method of claim 38, wherein said vector comprises a viral vector.

43. The method of claim 42, wherein said viral vector is selected from the group consisting of retrovirus, adenovirus, herpesvirus, vaccinia virus, and adeno-associated virus.

44. The method of claim 42, wherein said viral vector is an adenoviral vector.

45. A method of inhibiting growth in a tumor, the method comprising contacting the tumor with an angiopoietin-1 polypeptide in an amount effective to inhibit said growth, wherein the tumor is a colon tumor, a colorectal tumor, or a liver tumor.

46. The method of claim 45, wherein said angiopoietin-1 polypeptide is introduced into said cell by the direct introduction of said angiopoietin-1 polypeptide.

47. The method of claim 45, wherein said angiopoietin-1 polypeptide is introduced into the cell through the introduction of an angiopoietin-1-encoding polynucleotide.

48. The method of claim 45, wherein the colon tumor or colorectal tumor is in a patient.

49. The method of claim 48, further comprising treating the tumor with a second agent, wherein the second agent is a therapeutic polypeptide, polynucleotide encoding a therapeutic polypeptide, chemotherapeutic agent, or radiotherapeutic agent.

50. The method of claim 48, wherein the angiopoietin-1 polypeptide is administered by injection into the patient.

51. The method of claim 47, wherein said polynucleotide is a deoxyribonucleic acid molecule that encodes an angiopoietin-1 polypeptide.

52. The method of claim 51, wherein said angiopoietin-1-encoding polynucleotide further comprises control sequences operatively linked to said angiopoietin-1 encoding polynucleotide.

53. The method of claim 52, wherein said angiopoietin-1-encoding polynucleotide is located on a vector.

54. The method of claim 51, wherein said polynucleotide is operably linked to a promoter.

55. The method of claim 54, wherein said promoter is selected from the group consisting of CMV IE, SV40 IE, RSV LTR, or β-actin.

56. The method of claim 53, wherein said vector comprises a plasmid vector.

57. The method of claim 53, wherein said vector comprises a viral vector.

58. The method of claim 57, wherein said viral vector is selected from the group consisting of retrovirus, adenovirus, herpesvirus, vaccinia virus, and adeno-associated virus.

59. The method of claim 57, wherein the viral vector is an adenoviral vector.
